(19) United States
(12) Patent Application Publication
(10) Pub. No.: US 2008/0175905 A1
(43) Pub. Date: Jul. 24, 2008

(54) ESTROGEN/SERM AND ESTROGEN/PROGESTIN BI-LAYER TABLETS

(75) Inventors: Xiuying Liu, Glen Rock, NJ (US); John Kresevic, New Windsor, NY (US); Nizamuddin Baksh, South Hackensack, NJ (US); Robin Enever, New City, NY (US)

Correspondence Address: WilmerHale/Wyeth, 60 STATE STREET, BOSTON, MA, 02109, US

(73) Assignee: Wyeth, Madison, NJ (US)

(21) Appl. No.: 11/946,586

(22) Filed: Nov. 28, 2007

Related U.S. Application Data
(60) Provisional application No. 60/867,715, filed on Nov. 29, 2006.

(51) Int. Cl. A61K 9/20; A61K 31/56; A61P 5/24

(52) U.S. Cl. 424/464; 514/169; 514/177

(57) ABSTRACT

The present invention is directed to bi-layer tablets comprising at least one estrogen in a first layer and a therapeutic agent in a second layer, and processes for their preparation.

### CROSS-REFERENCE TO RELATED APPLICATIONS

This application claims priority to provisional U.S. Application Ser. No. 60/867,715, filed on Nov. 29, 2006, which is herein incorporated by reference in its entirety.

### FIELD OF THE INVENTION

The invention is directed generally to the field of pharmaceutical formulations. More specifically, the invention relates to bi-layer compositions and methods of preparing such compositions. In some embodiments, the compositions comprises at least one estrogen in a first layer and at least one therapeutic agent in a second layer.

### BACKGROUND OF THE INVENTION

Menopause is generally defined as the last natural menstrual period and is characterized by the cessation of ovarian function, leading to the substantial diminution of circulating estrogen in the bloodstream. Menopause is usually identified, in retrospect, after 12 months of amenorrhea. It is usually not a sudden event, but is often preceded by a time of irregular menstrual cycles prior to eventual cessation of menses. Following the cessation of menstruation, the decline in endogenous estrogen concentrations is typically rapid. There is a decrease in serum estrogens from circulating levels ranging from 40-250 pg/mL of estradiol and 40-170 pg/mL of estrone during ovulatory cycles to less than 15 pg/mL of estradiol and 30 pg/mL of estrone in postmenopausal women.

As these estrogens decline during the time preceding (perimenopause) and following the menopause (postmenopause), various physiological changes may result, including vulvar and vaginal atrophy causing vaginal dryness, pruritus and dyspareunia, and vasomotor instability manifested as hot flushes. Other menopausal disturbances may include depression, insomnia, and nervousness. The long-term physiologic effects of postmenopausal estrogen deprivation may result in significant morbidity and mortality due to increase in the risk factors for cardiovascular disease and osteoporosis. Menopausal changes in blood lipid levels, a major component of the pathogenesis of coronary heart disease (CHD), may be precursors to increased incidence of ischemic heart disease, atherosclerosis, and other cardiovascular disease. A rapid decrease in bone mass of both cortical (spine) and trabecular (hip) bone can be seen immediately after the menopause, with a total bone mass loss of 1% to 5% per year, continuing for 10 to 15 years.

Estrogen replacement therapy (ERT) is beneficial for symptomatic relief of hot flushes and genital atrophy and for prevention of postmenopausal osteoporosis. ERT has been recognized as an advantageous treatment for relief of vasomotor symptoms. Long term ERT can prevent osteoporosis because it decreases bone loss, reduces spine and hip fracture, and prevents loss of height. In addition, ERT has been shown to be effective in increasing high density lipoprotein-cholesterol (HDL-C) and in reducing low density lipoprotein cholesterol (LDL-C), affording possible protection against CHD. ERT also can provide antioxidant protection against free radical mediated disorders or disease states. Estrogens have also been reported to confer neuroprotection, and inhibit neurodegenerative disorders, such as Alzheimer's disease (see U.S. Pat. No. 5,554,601, which is hereby incorporated by reference in its entirety).

The normal protocol for ERT calls for estrogen supplementation using such formulations containing estrone, estriol, ethynyl estradiol or conjugated estrogens isolated from natural sources (i.e. Premarin® conjugated estrogens from Wyeth). In some patients, therapy may be contraindicated due to the proliferative effects of unopposed estrogens have on uterine tissue. This proliferation is associated with increased risk for endometriosis and/or endometrial cancer. The effects of unopposed estrogens on breast tissue is less clear, but is of some concern. Accordingly, one trend has been towards the development of low dose treatment regimen that minimize the adverse effects of ERT.

Another approach has been to administer a progestin, either sequentially or in combination, with the estrogen. There are extensive clinical data showing that the relative risk of endometrial cancer can be reduced by the addition of a progestin to ERT. The addition of a progestin to estrogen therapy can help prevent estrogen-induced endometrial proliferation. With appropriate doses of daily estrogen and progestin, combined estrogen replacement therapy has been shown to be effective in relieving vaginal atrophy and vasomotor symptoms, preventing postmenopausal osteoporosis, and reducing the risk of endometrial cancer by prevention of endometrial hyperplasia.

A third approach to minimize the adverse effects of ERT is the use of selective estrogen receptor modulators (SERMs) in conjunction with ERT. Selective SERMs are a class of compounds that demonstrate an affinity for estrogen receptors (ER) but show tissue selective estrogenic effects. An example of a SERM is bazedoxifene acetate (1-[4-(2-azepan-1-yl-ethoxy)-benzyl]-2-(4-hydroxy-phenyl)-3-methyl-1H-indol-5-ol acetic acid), having the chemical formula shown below:

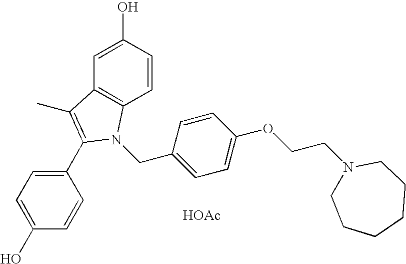

Bazedoxifene acetate has been reported to prevent bone loss and protect the cardiovascular system and reduce or eliminate the negative effects on the uterus and breast (potential risk of uterine and breast cancers). Consistent with its classification as a SERM, bazedoxifene acetate demonstrates little or no stimulation of uterine response in preclinical models of uterine stimulation. Conversely, bazedoxifene acetate demonstrates an estrogen agonist-like effect in preventing bone loss and reducing cholesterol in an ovariectomized rat model of osteopenia. In an MCF-7 cell line (human breast cancer cell line), bazedoxifene acetate behaves as an estrogen antagonist. These data demonstrate that bazedoxifene acetate is estrogenic on bone and cardiovascular lipid parameters and antiestrogenic on uterine and mammary tissue and thus has the potential for treating a number of different diseases or disease-like states wherein the estrogen receptor is involved.

In summary, a number of different approaches exist for minimizing the adverse side-effects of ERT, including the administration of a progestin or SERM in conjunction with ERT. Given the growing trend towards progestin/estrogen and SERM/estrogen therapies, there is an interest in developing a single dosage form which can deliver multiple drugs at different release rates. There is a particular need to develop drug delivery systems which can be tailored to give any desired treatment regimen, e.g., fast release of one drug and sustained release of another, sustained release of both drugs, sequential dosing regimens, continuous dosing regimens and the like, in order to improve clinical outcome. Additionally, there is a need to improve patient compliance by eliminating the need for separate administration of multiple drugs. This invention meets these needs and others.

### SUMMARY OF THE INVENTION

The present invention provides bi-layer tablets comprising:

(a) a first layer comprising at least one estrogen; and

(b) a second layer comprising one or more therapeutic agents selected from the group consisting of a selective estrogen receptor modulator and a progestational agent.

In some embodiments, the present invention provides bi-layer tablets wherein
the estrogen comprises conjugated estrogens; and
the second layer comprises one or more therapeutic agents selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

In some embodiments, the present invention provides bi-layer tablets wherein the first layer and the second layer each further independently comprise a hydrophilic gel-forming polymer component. In some cases, the hydrophilic polymer is present in only one of the layers.

In certain embodiments, the invention relates to a bi-layer tablet having a hydrophilic gel-forming polymer component in one layer that comprises 5% to 80% by weight of the layer. In some embodiments, the bi-layer tablet has a hydrophilic gel-forming polymer component in one layer that comprises 1% to 40% by weight of the layer.

In some embodiments, the present invention provides bi-layer tablets wherein:
the hydrophilic gel-forming polymer component of the first layer comprises from about 5% to about 80% by weight of the first layer; and
the hydrophilic gel-forming polymer component of the second layer comprises from about 1% to about 40% by weight of the second layer.

In some embodiments, the present invention provides bi-layer tablets wherein:

(a) the first layer further comprises:
a filler/diluent component comprising from about 10% to about 90% by weight of the first layer;
a filler/binder component comprising from about 0.1% to about 30% by weight of the first layer; and
optionally, a lubricant component comprising from about 0.01% to about 3% by weight of the first layer; and

(b) the second layer further comprises:
a filler/diluent component comprising from about 10% to about 75% by weight of the second layer;
a filler/binder component comprising up to about 60% by weight of the second layer;
optionally, a lubricant component comprising from about 0.01% to about 3% by weight of the second layer;
optionally, a disintegrant comprising up to about 4% by weight of said second layer; and
optionally, an antioxidant component comprising from about 0.01% to about 4% by weight of said second layer.

In some embodiments, the present invention provides a tablet selected from a plurality of tablets of the invention, wherein the plurality has a mean dissolution profile wherein:

the mean of % of the estrogen released per tablet after 1, 2, 3, 4, and 5 hours under estrogen dissolution conditions is substantially equal to the sum of f, a*A, b*B, c*A², d*B², and e*A*B; and

the mean of % of the therapeutic agent per tablet released after 0.25, 0.5, 1, 2, and 6 hours under type I therapeutic agent dissolution conditions is substantially equal to the sum of m, n*A, o*B, p*A², q*B², and r*A*B;

wherein:

A is the % of hydrophilic gel-forming polymer by weight of the first layer;

B is the % of hydrophilic gel-forming polymer by weight of the second layer;

f, at 1 hour, is 84.405;

a, at 1 hour, is −1.801;

b, at 1 hour, is −3.141;

c, at 1 hour, is 0.0159;

d, at 1 hour, is 0.0991;

e, at 1 hour, is 0.00609;

f, at 2 hours, is 112.029;

a, at 2 hours, is −1.825;

b, at 2 hours, is −4.401;

c, at 2 hours, is 0.0134;

d, at 2 hours, is 0.131;

e, at 2 hours, is 0.00794;

f, at 3 hours, is 128.469;

a, at 3 hours, is −1.687;

b, at 3 hours, is −5.218;

c, at 3 hours, is 0.0105;

d, at 3 hours, is 0.153;

e, at 3 hours, is 0.00741;

f, at 4 hours, is 133.525;

a, at 4 hours, is −1.437;

b, at 4 hours, is −5.053;

c, at 4 hours, is 0.00776;

d, at 4 hours, is 0.152;

e, at 4 hours, is 0.000658;

f, at 5 hours, is 133.182;

a, at 5 hours, is −1.064;

b, at 5 hours, is −4.893;

c, at 5 hours, is 0.004363;

d, at 5 hours, is 0.1558;

e, at 5 hours, is −0.0076;

m, at 0.25 hour, is 94.7399;

n, at 0.25 hour, is −0.2561;

o, at 0.25 hour, is −10.7494;

p, at 0.25 hour, is −0.0038874;

q, at 0.25 hour, is 0.3088;

r, at 0.25 hour, is 0.02228;

m, at 0.5 hour, is 113.1339;

n, at 0.5 hour, is −0.2832;

o, at 0.5 hour, is −12.549;

p, at 0.5 hour, is −0.00428;

q, at 0.5 hour, is 0.35267;

r, at 0.5 hour, is 0.025698;

m, at 1 hour, is 133.966;

n, at 1 hour, is −0.446;

o, at 1 hour, is −14.0527;

p, at 1 hour, is −0.0021667;

q, at 1 hour, is 0.38816;

r, at 1 hour, is 0.02607;

m, at 2 hours, is 153.718;

n, at 2 hours, is −0.8427;

o, at 2 hours, is −14.196;

p, at 2 hours, is 0.003872;

q, at 2 hours, is 0.38144;

r, at 2 hours, is 0.023435;

m, at 6 hours, is 133.7326;

n, at 6 hours, is −1.134;

o, at 6 hours, is −4.458;

p, at 6 hours, is 0.0115;

q, at 6 hours, is 0.05789; and

r, at 6 hours, is 0.0006761.

The present invention further provides processes for producing the bi-layer tablets of the invention comprising compressing together:
a first mixture comprising at least one estrogen; and
a second mixture comprising one or more therapeutic agents selected from the group consisting of a selective estrogen receptor modulator and a progestational agent.

The present invention further provides products of the processes of the invention.

### DESCRIPTION OF THE FIGURES

FIG. 1 depicts the dissolution of medroxyprogesterone acetate over time from the bi-layer tablet of Example 15A (y-axis is % of released MPA, x-axis is time in hours). The data points and standard deviation for each point are shown in Table XVI for Example 15A.

FIG. 2 depicts the dissolution of medroxyprogesterone acetate over time from the bi-layer tablet of Example 15G (y-axis is % of released MPA, x-axis is time in hours). The data points and standard deviation for each point are shown in Table XVI for Example 15G.

FIG. 3 depicts the dissolution of medroxyprogesterone acetate over time from the bi-layer tablet of Example 15C (y-axis is % of released MPA, x-axis is time in Hours). The data points and standard deviation for each point are shown in Table XVI for Example 15C.

FIG. 4 depicts the dissolution of medroxyprogesterone acetate over time from the bi-layer tablet of Example 15F (y-axis is % of released MPA, x-axis is time in hours). The data points and standard deviation for each point are shown in Table XVI for Example 15F.

FIG. 5 depicts the dissolution of medroxyprogesterone acetate over time from the bi-layer tablet of Example 15H (y-axis is % of released MPA, x-axis is time in hours). The data points and standard deviation for each point are shown in Table XVI for Example 15H.

FIG. 6 depicts the dissolution of medroxyprogesterone acetate over time from the bi-layer tablet of Example 15D (y-axis is % of released MPA, x-axis is time in hours). The data points and standard deviation for each point are shown in Table XVI for Example 15D.

FIG. 7 depicts the dissolution of medroxyprogesterone acetate over time from the bi-layer tablet of Example 15B (y-axis is % of released MPA, x-axis is time in hours). The data points and standard deviation for each point are shown in Table XVI for Example 15B.

FIG. 8 depicts the dissolution of medroxyprogesterone acetate over time from the bi-layer tablet of Example 15E (y-axis is % of released MPA, x-axis is time in hours). The data points and standard deviation for each point are shown in Table XVI for Example 15E.

FIG. 9 depicts the dissolution of conjugated estrogens over time from the bi-layer tablet of Example 15A (y-axis is % of released CE, x-axis is time in hours). The data points and standard deviation for each point are shown in Table XVII for Example 15A.

FIG. 10 depicts the dissolution of conjugated estrogens over time from the bi-layer tablet of Example 15G (y-axis is % of released CE, x-axis is time in hours). The data points and standard deviation for each point are shown in Table XVII for Example 15G.

FIG. 11 depicts the dissolution of conjugated estrogens over time from the bi-layer tablet of Example 15C (y-axis is % of released CE, x-axis is time in hours). The data points and standard deviation for each point are shown in Table XVII for Example 15C.

FIG. 12 depicts the dissolution of conjugated estrogens over time from the bi-layer tablet of Example 15F (y-axis is % of released CE, x-axis is time in hours). The data points and standard deviation for each point are shown in Table XVII for Example 15F.

FIG. 13 depicts the dissolution of conjugated estrogens over time from the bi-layer tablet of Example 15H (y-axis is % of released CE, x-axis is time in hours). The data points and standard deviation for each point are shown in Table XVII for Example 15H.

FIG. 14 depicts the dissolution of conjugated estrogens over time from the bi-layer tablet of Example 15D (y-axis is % of released CE, x-axis is time in hours). The data points and standard deviation for each point are shown in Table XVII for Example 15D.

FIG. 15 depicts the dissolution of conjugated estrogens over time from the bi-layer tablet of Example 15B (y-axis is % of released CE, x-axis is time in hours). The data points and standard deviation for each point are shown in Table XVII for Example 15B.

FIG. 16 depicts the dissolution of conjugated estrogens over time from the bi-layer tablet of Example 15E (y-axis is % of released CE, x-axis is time in hours). The data points and standard deviation for each point are shown in Table XVII for Example 15E.

FIG. 17 depicts the dissolution of medroxyprogesterone acetate over time from the bi-layer tablet of Example 20A (y-axis is % of released MPA, x-axis is time in minutes). The data points and standard deviation for each point are shown in Table XXII for Example 20A.

FIG. 18 depicts the dissolution of medroxyprogesterone acetate over time from the bi-layer tablet of Example 20B (y-axis is % of released MPA, x-axis is time in minutes). The data points and standard deviation for each point are shown in Table XXII for Example 20B.

FIG. 19 depicts the dissolution of medroxyprogesterone acetate over time from the bi-layer tablet of Example 20C (y-axis is % of released MPA, x-axis is time in minutes). The data points and standard deviation for each point are shown in Table XXII for Example 20C.

FIG. 20 depicts the dissolution of medroxyprogesterone acetate over time from the bi-layer tablet of Example 20D (y-axis is % of released MPA, x-axis is time in minutes). The data points and standard deviation for each point are shown in Table XXII for Example 20D.

FIG. 21 depicts the dissolution of medroxyprogesterone acetate over time from the bi-layer tablet of Example 20E (y-axis is % of released MPA, x-axis is time in minutes). The data points and standard deviation for each point are shown in Table XXII for Example 20E.

FIG. 22 depicts the dissolution of medroxyprogesterone acetate over time from the bi-layer tablet of Example 20F (y-axis is % of released MPA, x-axis is time in minutes). The data points and standard deviation for each point are shown in Table XXII for Example 20F.

FIG. 23 depicts the dissolution of medroxyprogesterone acetate over time from the bi-layer tablet of Example 20G (y-axis is % of released MPA, x-axis is time in minutes). The data points and standard deviation for each point are shown in Table XXII for Example 20G.

FIG. 24 depicts the dissolution of medroxyprogesterone acetate over time from the bi-layer tablet of Example 20H (y-axis is % of released MPA, x-axis is time in minutes). The data points and standard deviation for each point are shown in Table XXII for Example 20H.

FIG. 25 depicts the dissolution of medroxyprogesterone acetate over time from the bi-layer tablet of Example 20I (y-axis is % of released MPA, x-axis is time in minutes). The data points and standard deviation for each point are shown in Table XXII for Example 20I.

FIG. 26 depicts the dissolution of medroxyprogesterone acetate over time from the bi-layer tablet of Example 20J (y-axis is % of released MPA, x-axis is time in hours). The data points and standard deviation for each point are shown in Table XXVI for Example 20J.

FIG. 27 depicts the dissolution of medroxyprogesterone acetate over time from the bi-layer tablet of Example 20K (y-axis is % of released MPA, x-axis is time in hours). The data points and standard deviation for each point are shown in Table XXVI for Example 20K.

FIG. 28 depicts the dissolution of medroxyprogesterone acetate over time from the bi-layer tablet of Example 20L (y-axis is % of released MPA, x-axis is time in hours). The data points and standard deviation for each point are shown in Table XXVI for Example 20L.

FIG. 29 depicts the dissolution of medroxyprogesterone acetate over time from the bi-layer tablet of Example 20M (y-axis is % of released MPA, x-axis is time in hours). The data points and standard deviation for each point are shown in Table XXVI for Example 20M.

FIG. 30 depicts the dissolution of medroxyprogesterone acetate over time from the bi-layer tablet of Example 20N (y-axis is % of released MPA, x-axis is time in hours). The data points and standard deviation for each point are shown in Table XXVI for Example 20N.

FIG. 31 depicts the dissolution of medroxyprogesterone acetate over time from the bi-layer tablet of Example 20O (y-axis is % of released MPA, x-axis is time in hours). The data points and standard deviation for each point are shown in Table XXVI for Example 20O.

FIG. 32 depicts the dissolution of conjugated estrogens over time from the bi-layer tablet of Example 20A (y-axis is % of released CE, x-axis is time in hours). The data points and standard deviation for each point are shown in Table XXI for Example 20A.

FIG. 33 depicts the dissolution of conjugated estrogens over time from the bi-layer tablet of Example 20B (y-axis is % of released CE, x-axis is time in hours). The data points and standard deviation for each point are shown in Table XXI for Example 20B.

FIG. 34 depicts the dissolution of conjugated estrogens over time from the bi-layer tablet of Example 20C (y-axis is % of released CE, x-axis is time in hours). The data points and standard deviation for each point are shown in Table XXI for Example 20C.

FIG. 35 depicts the dissolution of conjugated estrogens over time from the bi-layer tablet of Example 20D (y-axis is % of released CE, x-axis is time in hours). The data points and standard deviation for each point are shown in Table XXI for Example 20D.

FIG. 36 depicts the dissolution of conjugated estrogens over time from the bi-layer tablet of Example 20E (y-axis is % of released CE, x-axis is time in hours). The data points and standard deviation for each point are shown in Table XXI for Example 20E.

FIG. 37 depicts the dissolution of conjugated estrogens over time from the bi-layer tablet of Example 20F (y-axis is % of released CE, x-axis is time in hours). The data points and standard deviation for each point are shown in Table XXI for Example 20F.

FIG. 38 depicts the dissolution of conjugated estrogens over time from the bi-layer tablet of Example 20G (y-axis is % of released CE, x-axis is time in hours). The data points and standard deviation for each point are shown in Table XXI for Example 20G

FIG. 39 depicts the dissolution of conjugated estrogens over time from the bi-layer tablet of Example 20H (y-axis is % of released CE, x-axis is time in hours). The data points and standard deviation for each point are shown in Table XXI for Example 20H.

FIG. 40 depicts the dissolution of conjugated estrogens over time from the bi-layer tablet of Example 20I (y-axis is % of released CE, x-axis is time in hours). The data points and standard deviation for each point are shown in Table XXI for Example 20I.

FIG. 41 depicts the dissolution of conjugated estrogens over time from the bi-layer tablet of Example 20J (y-axis is % of released CE, x-axis is time in hours). The data points and standard deviation for each point are shown in Table XXV for Example 20J.

FIG. 42 depicts the dissolution of conjugated estrogens over time from the bi-layer tablet of Example 20K (y-axis is % of released CE, x-axis is time in hours). The data points and standard deviation for each point are shown in Table XXV for Example 20K.

FIG. 43 depicts the dissolution of conjugated estrogens over time from the bi-layer tablet of Example 20L (y-axis is % of released CE, x-axis is time in hours). The data points and standard deviation for each point are shown in Table XXV for Example 20L.

FIG. 44 depicts the dissolution of conjugated estrogens over time from the bi-layer tablet of Example 20M (y-axis is % of released CE, x-axis is time in hours). The data points and standard deviation for each point are shown in Table XXV for Example 20M.

FIG. 45 depicts the dissolution of conjugated estrogens over time from the bi-layer tablet of Example 20N (y-axis is % of released CE, x-axis is time in hours). The data points and standard deviation for each point are shown in Table XXV for Example 20N.

FIG. 46 depicts the dissolution of conjugated estrogens over time from the bi-layer tablet of Example 20O (y-axis is % of released CE, x-axis is time in hours). The data points and standard deviation for each point are shown in Table XXV for Example 20O.

FIG. 47 depicts the dissolution of bazedoxifene acetate over time from the bi-layer tablet of Example 28A (y-axis is % of released BZA, x-axis is time in minutes). The data points and standard deviation for each point are shown in Table XXXIV for Example 28A.

FIG. 48 depicts the dissolution of bazedoxifene acetate over time from the bi-layer tablet of Example 28B (y-axis is % of released BZA, x-axis is time in minutes). The data points and standard deviation for each point are shown in Table XXXIV for Example 28B.

FIG. 49 depicts the dissolution of bazedoxifene acetate over time from the bi-layer tablet of Example 28C (y-axis is % of released BZA, x-axis is time in minutes). The data points and standard deviation for each point are shown in Table XXXIV for Example 28C.

FIG. 50 depicts the dissolution of conjugated estrogens over time from the bi-layer tablet of Example 28A (y-axis is % of released CE, x-axis is time in minutes). The data points and standard deviation for each point are shown in Table XXXV for Example 28A.

FIG. 51 depicts the dissolution of conjugated estrogens over time from the bi-layer tablet of Example 28B (y-axis is % of released CE, x-axis is time in minutes). The data points and standard deviation for each point are shown in Table XXXV for Example 28B.

FIG. 52 depicts the dissolution of conjugated estrogens over time from the bi-layer tablet of Example 28C (y-axis is % of released CE, x-axis is time in minutes). The data points and standard deviation for each point are shown in Table XXXV for Example 28C.

FIG. 53 is a graph depicting the % of BZA released over time for Batch L34419-55 (see Table XLV, Batch L34419-55 for each data point and the associated standard deviation).

FIG. 54 is a graph depicting the % of BZA released over time for Batch L34419-56 (see Table XLV, Batch L34419-56 for each data point and the associated standard deviation).

FIG. 55 is a graph depicting the % of BZA released over time for Batch L34419-57 (see Table XLV, Batch L34419-57 for each data point and the associated standard deviation).

FIG. 56 is a graph depicting the % of BZA released over time for Batch L34419-62 (see Table XLV, Batch L34419-62 for each data point and the associated standard deviation).

FIG. 57 is a graph depicting the % of BZA released over time for Batch L34419-63 (see Table XLV, Batch L34419-63 for each data point and the associated standard deviation).

FIG. 58 is a graph depicting the % of BZA released over time for Batch L34419-64 (see Table XLV, Batch L34419-64 for each data point and the associated standard deviation).

FIG. 59 is a graph depicting the % of BZA released over time for Batch L34419-65 (see Table XLV, Batch L34419-65 for each data point and the associated standard deviation).

FIG. 60 is a graph depicting the % of CE released over time for Batch L34419-55 (see Table XLVI, Batch L34419-55 for each data point and the associated standard deviation).

FIG. 61 is a graph depicting the % of CE released over time for Batch L34419-56 (see Table XLVI, Batch L34419-56 for each data point and the associated standard deviation).

FIG. 62 is a graph depicting the % of CE released over time for Batch L34419-57 (see Table XLVI, Batch L34419-57 for each data point and the associated standard deviation).

FIG. 63 is a graph depicting the % of CE released over time for Batch L34419-62 (see Table XLVI, Batch L34419-62 for each data point and the associated standard deviation).

FIG. 64 is a graph depicting the % of CE released over time for Batch L34419-63 (see Table XLVI, Batch L34419-63 for each data point and the associated standard deviation).

FIG. 65 is a graph depicting the % of CE released over time for Batch L34419-64 (see Table XLVI, Batch L34419-64 for each data point and the associated standard deviation).

FIG. 66 is a graph depicting the % of CE released over time for Batch L34419-64 (see Table XLVI, Batch L34419-64 for each data point and the associated standard deviation).

### DETAILED DESCRIPTION OF THE INVENTION

The present invention relates to a bi-layer tablet having improved characteristics, including content uniformity (C.U.), compared to compositions containing similar compounds such as compositions having one or more active layers coated via suspension layering or sugar coating The invention therefore includes methods for producing and testing such tablets, e.g., a tablet that includes a layer containing an estrogen and a different layer containing a selective estrogen receptor modulator (SERM) or a progestational agent. Because of the excellent content uniformity of each layer of the bi-layer tablet, e.g., the delivery of each active pharmaceutical ingredient (API) is improved, e.g., compared to a formulation in which the estrogen and SERM or progestin are compounded together. Typically, a bi-layer tablet as described herein will, with respect to C.U., have a risk specific dose (RSD) of less than 3% RSD, at or below 2% RSD, or at or below 1% RSD. In part, because the compounds are formulated in separate layers of the bi-layer tablet, the composition of each compound in the tablet can be formulated to provide C.U. that is specific for each compound. This is an improvement over currently available compositions of an estrogen and SERM or progestin in which the compounds are formulated together and therefore the composition represents a C.U. that is not specifically titrated (e.g., optimized) separately for each compound in the composition. In addition, the disclosed bi-layer tablets can be readily manufactured, e.g., with varying dosages of each compound, therefore adapting various formulations for specific intended uses, e.g., for treating infertility, perimenopause, menopause, and postmenopausal symptoms. The estrogen/SERM and estrogen/progestin tablets described herein thus have better tablet to tablet control than compositions that are currently available and therefore can provide better treatment for patients using such compositions. Additional advantages conferred because of the finding that the compositions described herein can be formulated to make an effective composition with C.U. that is generally improved over currently available compositions include; the ease of production of a bi-layer tablet comprising an estrogen and a SERM or progestin, it is commercially practical to make such tablets, including more economical, e.g., because the manufacturing time for tableting is less than for preparing an active coating drug. Finally, the methods and compositions provided herein permit titration of the bi-layer, which is advantageous for readily testing different in vitro release characteristics, which can result in different in vivo outcomes depending on how the excipients are titrated in the chosen composition.

### DEFINITIONS

As used herein, the term “about” means plus or minus 10% of the value, unless otherwise indicated herein.

As used herein, the term “alginic acid” refers to a naturally occurring hydrophilic colloidal polysaccharide obtained from the various species of seaweed, or synthetically modified polysaccharides thereof.

As used herein, the term “sodium alginate” refers to a sodium salt of alginic acid and can be formed by reaction of alginic acid with a sodium containing base such as sodium hydroxide or sodium carbonate. As used herein, the term “potassium alginate” refers to a potassium salt of alginic acid and can be formed by reaction of alginic acid with a potassium containing base such as potassium hydroxide or potassium carbonate. As used herein, the term “calcium alginate” refers to a calcium salt of alginic acid and can be formed by reaction of alginic acid with a calcium containing base such as calcium hydroxide or calcium carbonate. Suitable sodium alginates, calcium alginates, and potassium alginates include, but are not limited to, those described in R. C. Rowe and P. J. Shesky, Handbook of pharmaceutical excipients, (2006), 5th ed., which is incorporated herein by reference in its entirety. Suitable sodium alginates, include, but are not limited to, Kelcosol (available from ISP), Kelfone LVCR and HVCR (available from ISP), Manucol (available from ISP), and Protanol (available from FMC Biopolymer).

As used herein, the phrase “apparent viscosity” refers to a viscosity measured by the USP method.

As used herein, the term “calcium phosphate” refers to monobasic calcium phosophate, dibasic calcium phosphate or tribasic calcium phosphate.

Cellulose, cellulose floc, powdered cellulose, microcrystalline cellulose, silicified microcrystalline cellulose, carboxymethylcellulose, hydroxyethylcellulose, hydroxypropylcellulose, hydroxypropylmethylcellulose, and methylcellulose include, but are not limited to, those described in R. C. Rowe and P. J. Shesky, Handbook of pharmaceutical excipients, (2006), 5th ed., which is incorporated herein by reference in its entirety. As used herein, cellulose refers to natural cellulose. The term “cellulose” also refers to celluloses that have been modified with regard to molecular weight and/or branching, particularly to lower molecular weight. The term “cellulose” further refers to celluloses that have been chemically modified to attach chemical functionality such as carboxy, hydroxyl, hydroxyalkylene, or carboxyalkylene groups. As used herein, the term “carboxyalkylene” refers to a group of formula -alkylene-C(O)OH, or salt thereof. As used herein, the term “hydroxyalkylene” refers to a group of formula -alkylene-OH.

Suitable powdered celluloses for use in the invention include, but are not limited to Arbocel (available from JRS Pharma), Sanacel (available from CFF GmbH), and Solka-Floc (available from International Fiber Corp.).

Suitable microcrystalline celluloses include, but are not limited to, the Avicel pH series (available from FMC Biopolymer), Celex (available from ISP), Celphere (available from Asahi Kasei), Ceolus KG (available from Asahi Kasei), and Vivapur (available from JRS Pharma). In some embodiments, the microcrystalline cellulose is Avicel pH200.

As used herein, the term “carboxymethylcellulose” refers to a cellulose ether with pendant carboxymethyl groups of formula HO—C(O)—CH2-, attached to the cellulose via an ether linkage. Suitable carboxymethylcellulose calcium polymers include, but are not limited to, Nymel ZSC (available from Noviant).

As used herein, the term “hydroxyethylcellulose” refers to a cellulose ether with pendant hydroxyethyl groups of formula HO—CH2—CH2-, attached to the cellulose via an ether linkage. Suitable hydroxyethylcelluloses include, but are not limited to, Cellosize HEC (available from DOW), Natrosol (available from Hercules), and Tylose PHA (available from Clariant).

As used herein, the term “hydroxypropylcellulose” refers to a cellulose that has pendant hydroxypropoxy groups, and includes both high- and low-substituted hydroxypropylcellulose. In some embodiments, the hydroxypropylcellulose has about 5% to about 25% hydroxypropyl groups. Suitable hydroxypropylcelluloses include, but are not limited to, the Klucel series (available from Hercules), the Methocel series (available from Dow), the Nisso HPC series (available from Nisso), the Metolose series (available from Shin Etsu), and the LH series, including LHR-11, LH-21, LH-31, LH-20, LH-30, LH-22, and LH-32 (available from Shin Etsu).

As used herein, the term “methyl cellulose” refers to a cellulose that has pendant methoxy groups. Suitable methyl celluloses include, but are not limited to Culminal MC (available from Hercules).

As used herein, the term “carmellose calcium” refers to a crosslinked polymer of carboxymethylcellulose calcium.

As used herein, the term “copovidone” refers to a copolymer of vinylpyrrolidone and vinyl acetate, wherein the vinyl acetate monomers may be partially hydrolyzed. Suitable copovidone polymers include, but are not limited to Kollidon VA 64 (available from BASF, Luviskol VA (available from BASF, Plasdone S-630 (available from ISP), and Majsao CT (available from Cognis).

As used herein, the term “croscarmellose sodium” refers to a crosslinked polymer of carboxymethylcellulose sodium. In some embodiments, the croscarmellose sodium is Ac.Di.Sol (available from FMC Biopolymers).

As used herein, the term “crospovidone” refers to a crosslinked polymer of polyvinylpyrrolidone. Suitable crospovidone polymers include, but are not limited to Polyplasdone XL-10 (available from ISP) and Kollidon CL and CL-M (available from BASF).

As used herein, the phrase “dissolution profile” refers to the percentage of the total active pharmacological agent is a tablet that dissolves under specified conditions in a specified period of time.

As used herein, the term “fatty acid”, employed alone or in combination with other terms, refers to an aliphatic acid that is saturated or unsaturated. In some embodiments, the fatty acid in a mixture of different fatty acids. In some embodiments, the fatty acid has between about eight to about thirty carbons on average. In some embodiments, the fatty acid has about eight to about twenty-four carbons on average. In some embodiments, the fatty acid has about twelve to about eighteen carbons on average. Suitable fatty acids include, but are not limited to, stearic acid, lauric acid, myristic acid, erucic acid, palmitic acid, palmitoleic acid, capric acid, caprylic acid, oleic acid, linoleic acid, linolenic acid, hydroxystearic acid, 12-hydroxystearic acid, cetostearic acid, isostearic acid, sesquioleic acid, sesqui-9-octadecanoic acid, sesquiisooctadecanoic acid, benhenic acid, isobehenic acid, and arachidonic acid, or mixtures thereof.

As used herein, the term “fatty acid ester” refers to a compound formed between a fatty acid and a hydroxyl containing compound. In some embodiments, the fatty acid ester is a sugar ester of fatty acid. In some embodiments, the fatty acid ester is a glyceride of fatty acid. In some embodiments, the fatty acid ester is an ethoxylated fatty acid ester.

As used herein, the term “fatty alcohol”, employed alone or in combination with other terms, refers to an aliphatic alcohol that is saturated or unsaturated. In some embodiments, the fatty alcohol in a mixture of different fatty alcohols. In some embodiments, the fatty alcohol has between about eight to about thirty carbons on average. In some embodiments, the fatty alcohol has about eight to about twenty-four carbons on average. In some embodiments, the fatty alcohol has about twelve to about eighteen carbons on average. Suitable fatty alcohols include, but are not limited to, stearyl alcohol, lauryl alcohol, palmityl alcohol, palmitolyl acid, cetyl alcohol, capryl alcohol, caprylyl alcohol, oleyl alcohol, linolenyl alcohol, arachidonic alcohol, behenyl alcohol, isobehenyl alcohol, selachyl alcohol, chimyl alcohol, and linoleyl alcohol, or mixtures thereof.

As used herein, the term “gelatin” refers to any material derived from boiling the bones, tendons, and/or skins of animals, or the material known as agar, derived from seaweed. The term “gelatin” also refers to any synthetic modifications of natural gelatin. Suitable gelatins include, but are not limited to, Byco (available from Croda Chemicals) and Cryogel and Instagel (available from Tessenderlo), and the materials described in R. C. Rowe and P. J. Shesky, Handbook of pharmaceutical excipients, (2006), 5th ed., which is incorporated herein by reference in its entirety.

As used herein, the term “gum arabic” refers to natural, or synthetically modified, arabic gum. As used herein, the term “gum tragacanath” refers to natural, or synthetically modified, tragacanath gum. As used herein, the term “gum acacia” refers to natural, or synthetically modified, acacia gum. Suitable gum arabic, gum tragacanath, and gum acacia include, but are not limited to, those described in R. C. Rowe and P. J. Shesky, Handbook of pharmaceutical excipients, (2006), 5th ed., which is incorporated herein by reference in its entirety.

Suitable mannitols include, but are not limited to, PharmMannidex (available from Cargill), Pearlitol (available from Roquette), and Mannogem (available from SPI Polyols).

As used herein, the phrase “mean dissolution profile” means that the percentage of each active pharmacological agent which dissolves after specified period of time under specified conditions is first measured for each tablet in a plurality. The mean percentage of active pharmacological agent released at a given time for the plurality is then calculated by adding the percentages of active pharmacological agent released at a given time for each tablet and then dividing by the number of tablets in the plurality.

As used herein, the phrase “mean of % of the estrogen released per tablet” means that the percentage of estrogen which dissolves after specified period of time under specified conditions is first measured for each tablet in a plurality. The mean percentage of estrogen released at a given time for the plurality is then calculated by adding the percentages of estrogen released at a given time for each tablet and then dividing by the number of tablets in the plurality.

As used herein, the phrase “mean of % of the therapeutic agent released per tablet” means that the percentage of one of the therapeutic agents which dissolves after specified period of time under specified conditions is first measured for each tablet in a plurality. The mean percentage of therapeutic agent released at a given time for the plurality is then calculated by adding the percentages of the therapeutic agent released at a given time for each tablet and then dividing by the number of tablets in the plurality.

As used herein, the term “metallic alkyl sulfate” refers to a metallic salt formed between inorganic base and an alkyl sulfate compound. In some embodiments, the metallic alkyl sulfate has about eight carbons to about eighteen carbons. In some embodiments, metallic alkyl sulfate is a metallic lauryl sulfate. In some embodiments, the metallic alkyl sulfate is sodium lauryl sulfate.

As used herein, the term “metal carbonate” refers to any metallic carbonate, including, but not limited to sodium carbonate, calcium carbonate, and magnesium carbonate, and zinc carbonate.

As used herein, the term “metallic stearate” refers to a metal salt of stearic acid. In some embodiments, the metallic stearate is calcium stearate, zinc stearate, or magnesium stearate. In some embodiments, the metallic stearate is magnesium stearate.

As used herein, the term “mineral oil” refers to both unrefined and refined (light) mineral oil. Suitable mineral oils include, but are not limited to, the Avatech™ grades (available from Avatar Corp.), Drakeol™ grades (available from Penreco), Sirius™ grades (available from Shell), and the Citation™ grades (available from Avater Corp.).

As used herein, the term “plurality” refers to six or more tablets. In some embodiments, the plurality is derived from a single manufacturing batch of tablets.

As used herein, the term “polyvinyl alcohol” refers to a polymer formed by partial or complete hydrolysis of polyvinyl acetate. Suitable polyvinyl alcohols include, but are not limited to, the Airvol series (available from Air Products), the Alcotex series (available from Synthomer), the Elvanol series (available from DuPont), the Gelvatol series (available from Burkard), and the Gohsenol series (available from Gohsenol).

As used herein, the term “polyvinylpyrrolidone” refers to a polymer of vinylpyrrolidone. In some embodiments, the polyvinylpyrrolidone contains one or more additional polymerized monomers. In some embodiments, the additional polymerized monomer is a carboxy containing monomer. In some embodiments, the polyvinylpyrrolidone is povidone. In some embodiments, the polyvinylpyrrolidone has a molecular weight between 2500 and 3 million. In some embodiments, the polyvinylpyrrolidone is povidone K12, K17, K25, K30, K60, K90, or K120. Suitable polyvinylpyrrolidone polymers include, but are not limited to, the Kollidone™ series (available from BASF) and the Plasdone™ series (available from ISP).

As used herein, the term “propylene glycol fatty acid ester” refers to an monoether or diester, or mixtures thereof, formed between propylene glycol or polypropylene glycol and a fatty acid. Fatty acids that are useful for deriving propylene glycol fatty alcohol ethers include, but are not limited to, those defined herein. In some embodiments, the monoester or diester is derived from propylene glycol. In some embodiments, the monoester or diester has about 1 to about 200 oxypropylene units. In some embodiments, the polypropylene glycol portion of the molecule has about 2 to about 100 oxypropylene units. In some embodiments, the monoester or diester has about 4 to about 50 oxypropylene units. In some embodiments, the monoester or diester has about 4 to about 30 oxypropylene units. Suitable propylene glycol fatty acid esters include, but are not limited to, propylene glycol laurates: Lauroglycol™ FCC and 90 (available from Gattefosse); propylene glycol caprylates: Capryol™ PGMC and 90 (available from Gatefosse); and propylene glycol dicaprylocaprates: Labrafac™ PG (available from Gatefosse).

As used herein, the term “pharmaceutically acceptable salt” refers to a salt formed by the addition of a pharmaceutically acceptable acid or base to a compound disclosed herein. As used herein, the phrase “pharmaceutically acceptable” refers to a substance that is acceptable for use in pharmaceutical applications from a toxicological perspective and does not adversely interact with the active ingredient. Pharmaceutically acceptable salts, including mono- and bi-salts, include, but are not limited to, those derived from organic and inorganic acids such as, but not limited to, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in their entireties.

As used herein, the term “progestational agent” refers to progestagens and progestins.

As used herein, “released” means dissolved under the specified conditions.

As used herein, the term “selective estrogen receptor modulator” is a pharmacological agent with an affinity for the estrogen receptor, that in some tissues act like an estrogen but block estrogen action in other tissues.

Suitable sorbitols include, but are not limited to, PharmSorbidex E420 (available from Cargill), Liponic 70-NC and 76-NC (available from Lipo Chemical), Neosorb (available from Roquette), Partech SI (available from Merck), and Sorbogem (available from SPI Polyols).

Starch, sodium starch glycolate, and pregelatinized starch include, but are not limited to, those described in R. C. Rowe and P. J. Shesky, Handbook of pharmaceutical excipients, (2006), 5th ed., which is incorporated herein by reference in its entirety.

As used herein, the term “starch” refers to any type of natural or modified starch including, but not limited to, maize starch (also known as corn starch or maydis amylum), potato starch (also known as solani amylum), rice starch (also known as oryzae amylum), wheat starch (also known as tritici amylum), and tapioca starch. The term “starch” also refers to starches that have been modified with regard to molecular weight and branching. The term “starch” further refers to starches that have been chemically modified to attach chemical functionality such as carboxy, hydroxyl, hydroxyalkylene, or carboxyalkylene groups. As used herein, the term “carboxyalkylene” refers to a group of formula -alkylene-C(O)OH, or salt thereof. As used herein, the term “hydroxyalkylene” refers to a group of formula -alkylene-OH.

Suitable sodium starch glycolates include, but are not limited to, Explotab (available from JRS Pharma), Glycolys (available from Roquette), Primojel (available from DMV International), and Vivastar (available from JRS Pharma).

Suitable pregelatinized starches include, but are not limited to, Lycatab C and PGS (available from Roquette), Merigel (available from Brenntag), National 78-1551 (available from National Starch), Spress B820 (available from GPC), and Starch 1500 (available from Colorcon).

As used herein, the phrase “substantially equal to” means plus or minus 20% of the value.

As used herein, the term “substantially as shown” means that the profile is within plus or minus 2σ (twice the standard deviation) of the value for each point of the specified figure (the standard deviation, σ, for the individual points in the figures are shown in Tables XVI, XVII, XXI, XXII, XXV, XXVI, XXXIV, and XXXV). For example, in some embodiments, the profile is within plus or minus 1.9σ, 1.8σ, 1.7σ, 1.6σ, 1.5σ, 1.4σ, 1.3σ, 1.2σ, 1.1σ, or 1σ of the values at each point for a given figure.

As used herein, the phrase “under estrogen dissolution conditions” refers to subjecting a bi-layer tablet of the invention to USP Apparatus 2, at 50 rpm in 900 mL of 0.02 M sodium acetate buffer of pH 4.5, for 1, 2, 3, 4, and 5 hours, in order to measure the amount of estrogen which dissolves at each time. In some embodiments, the estrogen comprises conjugated estrogens.

As used herein, the phrase “under type I therapeutic agent conditions” refers to subjecting a bi-layer tablet of the invention to USP Apparatus 2, at 50 rpm in 900 mL of 0.54% sodium lauryl sulfate in water, for 6 hours, in order to measure the amount of therapeutic agent which dissolves at each time. In some embodiments, the therapeutic agent is medroxyprogesterone acetate.

As used herein, the phrase “under type II therapeutic agent conditions” refers to subjecting a bi-layer tablet of the invention to USP Apparatus 1 (basket), at 75 rpm in 900 mL of 10 mM acetic acid solution with 0.2% polysorbate 80 (Tween 80) at 37° C. for a period of 60 minutes, changing the speed to 250 rpm at 80 minutes, in order to measure the amount of therapeutic agent which dissolves at each time. In some embodiments, the therapeutic agent comprises bazedoxifene acetate.

As used herein, the term “vegetable oil” refers to naturally occurring or synthetic oils, which may be refined, fractionated or hydrogenated, including triglycerides. Suitable vegetable oils include, but are not limited to castor oil, hydrogenated castor oil, sesame oil, corn oil, peanut oil, olive oil, sunflower oil, safflower oil, soybean oil, benzyl benzoate, sesame oil, cottonseed oil, and palm oil. Other suitable vegetable oils include commercially available synthetic oils such as, but not limited to, Miglyol™ 810 and 812 (available from Dynamit Nobel Chicals, Sweden) Neobee™ M5 (available from Drew Chemical Corp.), Alofine™ (available from Jarchem Industries), the Lubritab™ series (available from JRS Pharma), the Sterotex™ (available from Abitec Corp.), Softisan™ 154 (available from Sasol), Croduret™ (available from Croda), Fancol™ (available from the Fanning Corp.), Cutina™ HR (available from Cognis), Simulsol™ (available from CJ Petrow), EmCon™ CO (available from Amisol Co.), Lipvol™ CO, SES, and HS-K (available from Lipo), and Sterotex™ HM (available from Abitec Corp.). Other suitable vegetable oils, including sesame, castor, corn, and cottonseed oils, include those listed in R. C. Rowe and P. J. Shesky, Handbook of pharmaceutical excipients, (2006), 5th ed., which is incorporated herein by reference in its entirety.

The present invention is directed to a bi-layer tablet comprising:

(a) a first layer comprising at least one estrogen; and

(b) a second layer comprising one or more therapeutic agents selected from the group consisting of a selective estrogen receptor modulator and a progestational agent.

In some embodiments, the estrogen comprises one or more of estradiol, estradiol benzoate, estradiol valerate, estriadiol cypionate, estradiol heptanoate, estradiol decanoate, estradiol acetate, estradiol diacetate, 17.alpha.-estradiol, ethinylestradiol, ethinylestradiol 3-acetate, ethinylestradiol 3-benzoate, estriol, estriol succinate, polyestrol phosphate, estrone, estrone acetate, estrone sulfate, piperazine estrone sulfate, quinestrol, mestranol, and conjugated equine estrogens, or other pharmaceutically acceptable ester and ether thereof. In some embodiments, the estrogen comprises conjugated estrogens. In some embodiments, the estrogen comprises combinations of estrogens.

As used herein, the term “conjugated estrogens” (“CE”) includes both natural and synthetic conjugated estrogens, such as the compounds described in the United States Pharmacopia (USP 23), as well as other estrogens so considered by those skilled in the art. Further, “conjugated estrogens” refers to esters of such compounds, such as the sulfate esters, salts of such compounds, such as sodium salts, and esters of the salts of such compounds, such as sodium salts of a sulfate ester, as well as other derivatives known in the art. Some specific examples include: 17-alpha and beta-dihydroequilin, equilenin, 17-alpha and beta-dihydroequilenin, estrone, 17-beta-estradiol, and their sodium sulfate esters.

Although CE are typically a mixture of estrogenic components, such as estrone and equilin, the first layer material may be formulated to either utilize such a mixture, or to include only selected or individual estrogenic components. These CE may be of synthetic or natural origin. Examples of synthetically produced estrogens include, inter alia, sodium estrone sulfate, sodium equilin sulfate, sodium 17α-dihydroequilin sulfate, sodium 17β-dihydroequilin sulfate, sodium 17α-estradiol sulfate, sodium 17β-estradiol sulfate, sodium equilenin sulfate, sodium 17α-dihydroequilenin sulfate, sodium 17β-dihydroequilenin sulfate, estropipate and ethinyl estradiol. The alkali metal salts of 8,9-dehydroestrone and the alkali metal salts of 8,9-dehydroestrone sulfate ester, as described in U.S. Pat. No. 5,210,081, which is herein incorporated by reference, also may be used. Naturally occurring CE are usually obtained from pregnant mare urine and then are processed and may be stabilized. Examples of such processes are set forth in U.S. Pat. Nos. 2,565,115 and 2,720,483, each of which are herein incorporated by reference.

Many CE products are commercially available. Preferred among these is the naturally occurring CE product known as Premarin® (Wyeth, Madison, N.J.). Another commercially available CE product prepared from synthetic estrogens is Cenestin® (Duramed Pharmaceuticals, Inc., Cincinnati, Ohio). The specific CE dose included in the first layer material may be any dosage required to achieve a specific therapeutic effect, and may vary depending on the specific treatment indicated, and on the specific CE included in the tablet. In some embodiments, the CE is a CE dessication with a sugar material such as lactose, sucrose, and the like. In some embodiments, the CE is a CE dessication with lactose.

In some embodiments, the progestational agent is selected from acetoxypregnenolone, allylestrenol, anagestone acetate, chlormadinone acetate, cyproterone, cyproterone acetate, desogestrel, dihydrogesterone, dimethisterone, ethisterone, ethynodiol diacetate, fluorogestone acetate, gestodene, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, progesterone, dienogest, drospirenone, nomegestrol acetate, hydroxyprogesterone, and trimegestone. In some embodiments, the progestational agent is medroxyprogesterone acetate or trimegestone. In some embodiments, the progestational agent is medroxyprogesterone acetate. In some embodiments, the progestational agent comprises combinations of progestational agents.

In some embodiments, the selective estrogen receptor modulator is TSE-424, ERA-923, raloxifene, tamoxifen, droloxifene, arzoxifene tamoxifen, raloxifene, toremifen, trioxifene, keoxifene, 4-hydroxytamoxifene, clomifene, nafoxidine, dihydroraloxifene, lasofoxifene, or 1-[4-(2-azepan-1-yl-ethoxy)-benzyl]-2-(4-hydroxy-phenyl)-3-methyl-1H-indol-5-ol; or pharmaceutically acceptable salt thereof. In some embodiments, the selective estrogen receptor modulator are those of U.S. Pat. Nos. 5,998,402 and 6,479,535, each of which is incorporated herein by reference in their entireties. In some embodiments, the selective estrogen receptor modulator is TSE-424, ERA-923, raloxifene, tamoxifen, droloxifene, arzoxifene, or 1-[4-(2-azepan-1-yl-ethoxy)-benzyl]-2-(4-hydroxy-phenyl)-3-methyl-1H-indol-5-ol; or pharmaceutically acceptable salt thereof. In some embodiments, the selective estrogen receptor modulator is raloxifene or 1-[4-(2-azepan-1-yl-ethoxy)-benzyl]-2-(4-hydroxy-phenyl)-3-methyl-1H-indol-5-ol; or pharmaceutically acceptable salt thereof. In some embodiments, the selective estrogen receptor modulator is 1-[4-(2-azepan-1-yl-ethoxy)-benzyl]-2-(4-hydroxy-phenyl)-3-methyl-1H-indol-5-ol, or pharmaceutically acceptable salt thereof. In some embodiments, the selective estrogen receptor modulator is 1-[4-(2-azepan-1-yl-ethoxy)-benzyl]-2-(4-hydroxy-phenyl)-3-methyl-1H-indol-5-ol, acetic acid salt. In some embodiments, the second layer comprises combinations of selective estrogen receptor modulators.

U.S. Pat. Nos. 5,998,402 and 6,479,535 report the preparation of 1-[4-(2-azepan-1-yl-ethoxy)-benzyl]-2-(4-hydroxy-phenyl)-3-methyl-1H-indol-5-ol, acetic acid salt (bazedoxifene acetate) and characterize the salt as having a melting point of 174-178° C. The synthetic preparation of bazedoxifene acetate has also appeared in the general literature. See, for example, Miller et al., J. Med. Chem., 2001, 44, 1654-1657, incorporated herein by reference in its entirety, which reports the salt as a crystalline solid having a melting point of 170.5-172.5° C. Further description of the drug's biological activity has appeared in the general literature as well (e.g. Miller, et al. Drugs of the Future, 2002, 27(2), 117-121), incorporated herein by reference in its entirety.

In some embodiments:
the estrogen comprises conjugated estrogens; and
the second layer comprises one or more therapeutic agents selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

The estrogen and therapeutic agents can also include pharmaceutically acceptable salts. In some embodiments, the estrogen comprises up to about 20%, up to about 15%, up to about 10%, up to about 9%, up to about 8%, up to about 7%, up to about 6%, up to about 5%, up to about 4%, up to about 3%, up to about 2%, up to about 1%, or up to about 0.5% by weight of the first layer. In some embodiments, the estrogen comprises from about 0.01 to about 1% by weight of the first layer.

In some embodiments, the one or more therapeutic agents comprise up to about 20%, up to about 15%, up to about 10%, up to about 9%, up to about 8%, up to about 7%, up to about 6%, up to about 5%, up to about 4%, up to about 3%, up to about 2%, up to about 1%, or up to about 1% by weight of the second layer. In some embodiments, the one or more therapeutic agents comprise from about 0.1% to about 1% by weight of the second layer. In some embodiments, the one or more therapeutic agents comprise from about 0.4% to about 0.8% by weight of the second layer. In some embodiments, the one or more therapeutic agents comprises from about 7% to about 8% by weight of the second layer.

In some embodiments:
the estrogen comprises from about 0.01% to about 2% by weight of the first layer; and
the one or more therapeutic agents comprise from about 0.01% to about 10% by weight of the second layer.

As used herein, the term “bi-layer tablet” refers a pharmaceutical dosage form comprising two portions that are contacted or compressed together along one surface. In some embodiments, the first layer comprises from about 20% to about 90%, from about 10% to about 70%, from about 10% to about 60%, from about 20% to about 50%, from about 20% to about 45%, from about 30% to about 40%, or from about 24% to about 32% by weight of the tablet. In some embodiments, the first layer comprises from about 20% to about 45% by weight of the tablet. In some embodiments, the first layer comprises about from about 28% to about 29% by weight of the tablet. In some embodiments, the first layer comprises about from about 30% to about 31% by weight of the tablet.

In some embodiments, the second layer comprises from about 20% to about 90%, from about 30% to about 90%, from about 40% to about 90%, from about 50% to about 85%, from about 55% to about 80%, from about 60% to about 70%, or from about 65% to about 75%, by weight of the tablet. In some embodiments, the first layer comprises from about 55% to about 80% by weight of the tablet. In some embodiments, the second layer comprises about from about 70% to about 71% by weight of the tablet. In some embodiments, the second layer comprises about from about 66% to about 67% by weight of the tablet.

In some embodiments, one or both of the first layer and the second layer each further independently comprise a hydrophilic gel-forming polymer component. As used herein, the term “hydrophilic gel-forming polymer component” refers to one or more hydrophilic polymers, wherein the dry polymer is capable of swelling in the presence aqueous media to form a highly viscous gelatinous mass. In some embodiments, the hydrophilic gel-forming polymer swells in a pH independent manner. In some embodiments, the hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum. In some embodiments, the hydrophilic gel-forming polymer component is hydroxypropylmethylcellulose (“HPMC”; also known as hypromellose). Suitable HPMC polymers include, but are not limited to the Methocel™ line of hydroxypropylmethylcellulose polymers such as Methocel Premium K100M CR, Methocel Premium K4M CR, and Methocel Premium K100 LV, available from Dow Chemical Company. In some embodiments, the hydrophilic gel-forming polymer component comprises HPMC K100M CR. In some embodiments, the hydrophilic gel-forming polymer component comprises HPMC K4M CR. In some embodiments, the hydrophilic gel-forming polymer component comprises HPMC K100M CR and HPMC K4M CR. In some embodiments, the hydrophilic gel-forming polymer component comprises HPMC K100M CR and HPMC K100 LV. In some embodiments, the hydrophilic gel-forming polymer component comprises a 1:1 mixture by weight of HPMC K100M CR and HPMC K4M. In some embodiments, the hydrophilic gel-forming polymer component comprises a 1:1 mixture by weight of HPMC K100M CR and HPMC K100 LV.

In some embodiments, the hydrophilic gel-forming polymer component comprises a hydroxypropylmethylcellulose polymer having from about 7% to about 12% by weight hydroxypropoxyl groups. In some embodiments, the hydrophilic gel-forming polymer component comprises a hydroxypropylmethylcellulose polymer having from about 19% to about 24% by weight methoxyl groups.

In some embodiments, the hydrophilic gel-forming polymer component comprises a hydroxypropylmethylcellulose polymer having an apparent viscosity from about 80 cP to about 150,000 cP. In some embodiments, the hydrophilic gel-forming polymer component comprises a hydroxypropylmethylcellulose polymer having an apparent viscosity from about 3000 to about 6000 cP. In some embodiments, the hydrophilic gel-forming polymer component comprises a hydroxypropylmethylcellulose polymer having an apparent viscosity from about 80 to about 120 cP. In some embodiments, the hydrophilic gel-forming polymer component comprises a hydroxypropylmethylcellulose polymer having an apparent viscosity from about 80,000 to about 120,000 cP. In some embodiments, the hydrophilic gel-forming polymer component comprises a 1:1 mixture by weight of a hydroxypropylmethylcellulose polymer having an apparent viscosity from about 80,000 to about 120,000 cP and a hydroxypropylmethylcellulose polymer having an apparent viscosity from about 3000 to about 6000 cP. In some embodiments, the hydrophilic gel-forming polymer component comprises a 1:1 mixture by weight of a hydroxypropylmethylcellulose polymer having an apparent viscosity from about 80,000 to about 120,000 cP and a hydroxypropylmethylcellulose polymer having an apparent viscosity from about 80 to about 120 cP. Apparent viscosity is determined by a Ubbelhode viscometer.

In some embodiments, the hydrophilic gel-forming polymer component of the first layer comprises from about 5% to about 80%, from about 5% to about 60%, from about 5% to about 15%, from about 15% to about 30%, from about 30% to about 40%, from about 40% to about 50%, from about 50% to about 60%, from about 22% to about 33%, or from about 40% to about 60% by weight of the first layer. In some embodiments, the hydrophilic gel-forming polymer component of the first layer comprises about 10%, about 20%, about 27.5%, about 35%, about 45%, about 55% by weight of the first layer.

In some embodiments, the hydrophilic gel-forming polymer component of the second layer comprises from about 1% to about 40%, from about 1% to about 8%, from about 8% to about 15%, from about 15% to about 30%, from about 2% to about 7%, from about 15% to about 25%, or from about 30% to about 40% by weight of the second layer. In some embodiments, the hydrophilic gel-forming polymer component of the second layer comprises about 5%, about 10%, or about 20 by weight of the second layer.

In some embodiments, the first layer further comprises a filler/diluent component. As used herein, the term “filler/diluent component” refers to one or more substances that act to dilute the active pharmacological agent to the desired dosage and/or that act as a carrier for the active pharmacological agent, although the substances may have additional, unspecified benefits. In some embodiments, the first filler/diluent component comprises one or more filler substances. In some embodiments, the first filler/diluent component comprises one or more diluent substances. In some embodiments, the first filler/diluent component is one or more substances that are diluents and fillers.

In some embodiments, the filler/diluent component of the first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate. In some embodiments, the filler/diluent component of the first layer comprises one or more of lactose, lactose monohydrate, mannitol, sucrose, maltodextrin, sorbitol, and xylitol. In some embodiments, the filler/diluent component of the first layer comprises one or more of lactose and lactose monohydrate. In some embodiments, the filler/diluent component of the first layer does not comprise sucrose.

In some embodiments, the second layer further optionally comprises a filler/diluent component. In some embodiments, the filler/diluent component of the second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate. In some embodiments, the filler/diluent component of the second layer comprises comprises one or more of lactose, lactose monohydrate, mannitol, sucrose, maltodextrin, sorbitol, and xylitol. In some embodiments, the filler/diluent component of the second layer comprises one or more of lactose and lactose monohydrate. In some embodiments, the filler/diluent component of the second layer does not comprise sucrose.

In some embodiments, the first layer further comprises a filler/binder component. As used herein, the term “filler/binder component” refers to one or more substances that can act as fillers and/or binders, although the substances may have additional, unspecified benefits. In some embodiments, the term “binder” refers to a substance that increases the mechanical strength and/or compressibility of a pharmaceutical composition comprising the pharmaceutical formulations of the invention. In some embodiments, the filler/binder component comprises one or more filler substances. In some embodiments, the filler/binder component comprises one or more binder substances. In some embodiments, the filler/binder comprises one or more substances that are fillers and binders. In some embodiments, filler/binder component of the first layer comprises one or more of microcrystalline cellulose, polyvinylpyrrolidone, copovidone, polyvinylalcohol, starch, gelatin, gum arabic, gum acacia, and gum tragacanth. In some embodiments, the filler/binder of the first layer comprises microcrystalline cellulose.

In some embodiments, the second layer further comprises a filler/binder. In some embodiments, the filler/binder of the second layer comprises one or more of microcrystalline cellulose, polyvinylpyrrolidone, copovidone, polyvinylalcohol, starch, gelatin, gum arabic, gum acacia, and gum tragacanth. In some embodiments, the filler/binder of the second layer comprises microcrystalline cellulose.

In some embodiments, the first layer further optionally comprises a lubricant component. As used herein, the term “lubricant component” refers to one or more substances that aids in preventing sticking to the equipment of the pharmaceutical formulations during processing and/or that improves powder flow of the formulation during processing. In some embodiments, the optional lubricant component of the first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol. In some embodiments, the optional lubricant component of the first layer, if present, comprises one or more of stearic acid, metallic stearate, sodium stearyl fumarate, glyceryl behenate, mineral oil, vegetable oil, and paraffin. In some embodiments, the optional lubricant component of the first layer, if present, comprises magnesium stearate.

In some embodiments, the second layer further optionally comprises a lubricant component. In some embodiments, the optional lubricant component of the second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol. In some embodiments, the optional lubricant component of the second layer, if present, comprises one or more of stearic acid, metallic stearate, sodium stearyl fumarate, glyceryl behenate, mineral oil, vegetable oil, and paraffin. In some embodiments, the optional lubricant component of the second layer, if present, comprises magnesium stearate.

In some embodiments, the first layer and the second layer each independently further comprise a filler/diluent component. In some embodiments the first layer and the second layer each independently further comprise a filler/binder component. In some embodiments the first layer and the second layer each independently further comprise an optional lubricant component.

In some embodiments, the second layer optionally further comprises a disintegrant. As used herein, the term “disintegrant component” refers to one or more substances that encourage disintegration in water (or water containing fluid in vivo) of a pharmaceutical composition comprising the pharmaceutical formulations of the invention. In some embodiments, the optional disintegrant of the second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose. In some embodiments, the optional disintegrant of the first layer comprises one or more of croscarmellose sodium, crospovidone, and sodium starch glycolate. In some embodiments, the optional disintegrant of the second layer comprises croscarmellose sodium.

In some embodiments, the second layer optionally further comprises an antioxidant component. The antioxidant component can be a single compound, such as ascorbic acid, or a mixture of antioxidants. A wide variety of antioxidant compound are known in the art, and are suitable for use in the present invention. Examples of such antioxidants that can be used in the present invention include vitamin E, vitamin E acetate (for example, dry vitamin E acetate 50% DC from BASF; also known as D,L-α-tocopheryl acetate) sodium ascorbate, ascorbyl palmitate, BHT (butylated hydroxytoluene) and BHA (butylated hydroxyanisole), each optionally in conjunction with an amount of ascorbic acid.

In some embodiments, the optional antioxidant component of the second layer, if present, comprises one or more of ascorbic acid, sodium ascorbate, ascorbyl palmitate, vitamin E, vitamin E acetate, butylated hydroxytoluene, and butylated hydroxyanisole. In some embodiments, the optional antioxidant component of the second layer, if present, comprises one or more of ascorbic acid, vitamin E, and vitamin E acetate. In some embodiments, the optional antioxidant component of the second layer, if present, comprises one or more of ascorbic acid and vitamin E acetate.

In some embodiments, the first layer comprises from about 10% to about 90%, from about 30% to about 70%, from about 50% to about 85%, from about 10% to about 50%, from about 45% to about 70%, or from about 40% to about 80% of a filler/diluent component by weight of the first layer. In some embodiments, the first layer comprises about 48.5%, about 56.9%, about 66%, about 74.4%, about 56%, about 64.4%, about 41%, about 49.4%, about 31%, about 39.4%, about 21%, or about 29.4% of a filler/diluent component by weight of the first layer.

In some embodiments, the second layer comprises from about 10% to about 75%, from about 25% to about 50%, from about 20% to about 60%, from about 35% to about 75%, from about 30% to about 50%, from about 35% to about 60%, from about 40% to about 70%, or from about 35% to about 70% of a filler/diluent component by weight of the second layer. In some embodiments, the second layer comprises about 39.1%, about 38.1%, about 49.1%, about 54.1%, about 46.2%, about 41.1%, about 30.9%, about 45.3%, about 40.3%, or about 30.3% of a filler/diluent component by weight of the second layer.

In some embodiments, the first layer further comprises from about 0.1% to about 30%, from about 1% to about 30%, from about 5% to about 25%, or from about 10% to about 20% of a filler/binder component by weight of the first layer. In some embodiments, the first layer further comprises about 15% of a filler/binder component by weight of the first layer.

In some embodiments, the second layer further comprises up to about 60% of a filler/binder component by weight of the second layer. In some embodiments, the second layer comprises from about 30%, from about 1% to about 30%, from about 5% to about 25%, or from about 10% to about 20% of a filler/binder component by weight of the second layer. In some embodiments, the second layer further comprises about 40% of a filler/binder component by weight of the second layer.

In some embodiments, the first layer further optionally comprises from about 0.01% to about 3%, from about 0.01% to about 2%, from about 0.01% to about 1%, or from about 0.1% to about 1% of a lubricant component by weight of the first layer. In some embodiments, the first layer further optionally comprises about 0.25% or about 0.5% of a lubricant component by weight of the first layer.

In some embodiments, the second layer further optionally comprises from about 0.01% to about 3σ, 0.01% to about 2σ, 0.01% to about 1%, or about 0.1% to about 1% of a lubricant component by weight of the second layer. In some embodiments, the second layer further optionally comprises about 0.25% or about 0.5% of a lubricant component by weight of the second layer.

In some embodiments, the second layer further optionally comprises up to about 4%, up to about 3%, or up to about 2% of a disintegrant by weight of the second layer. In some embodiments, the second layer further optionally comprises about 1% of a disintegrant by weight of the second layer.

In some embodiments, the second layer further optionally comprises from about 0.01% to about 4%, from about 0.01% to about 3%, or from about 0.01% to about 2% of an antioxidant component by weight of the second layer.

In some embodiments:
the hydrophilic gel-forming polymer component of the first layer comprises from about 5% to about 80% by weight of the first layer; and
the hydrophilic gel-forming polymer component of the second layer comprises from about 1% to about 40% by weight of the second layer.

In some embodiments:

(a) the first layer further comprises:
a filler/diluent component comprising from about 10% to about 90% by weight of the first layer;
a filler/binder component comprising from about 0.1% to about 30% by weight of the first layer; and
optionally, a lubricant component comprising from about 0.01% to about 3% by weight of the first layer; and

(b) the second layer further comprises:
a filler/diluent component comprising from about 10% to about 75% by weight of the second layer;
a filler/binder component comprising up to about 60% by weight of the second layer;
optionally, a lubricant component comprising from about 0.01% to about 3% by weight of the second layer;
optionally, a disintegrant comprising up to about 4% by weight of the second layer; and
optionally, an antioxidant component comprising from about 0.01% to about 4% by weight of the second layer.

In some embodiments:
the hydrophilic gel-forming polymer component of the first layer comprises from about 30% to about 40% by weight of the first layer; and
the hydrophilic gel-forming polymer component of the second layer comprises from about 15% to about 30% by weight of the second layer.

In some embodiments:

(a) the first layer further comprises:
a filler/diluent component comprising from about 30% to about 70% by weight of the first layer;
a filler/binder component comprising from about 5% to about 25% by weight of the first layer; and
optionally, a lubricant component comprising from about 0.01% to about 2% by weight of the first layer; and

(b) the second layer further comprises:
a filler/diluent component comprising from about 25% to about 50% by weight of the second layer;
a filler/binder component comprising from about 20% to about 60% by weight of the second layer;
optionally, a lubricant component comprising from about 0.01% to about 2% by weight of the second layer;
optionally, a disintegrant comprising up to about 4% by weight of the second layer; and
optionally, an antioxidant component comprising from about 0.01% to about 4% by weight of the second layer.

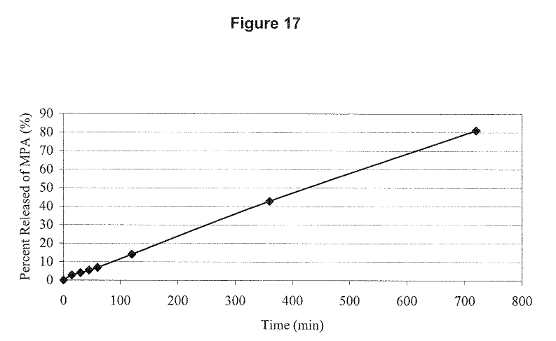

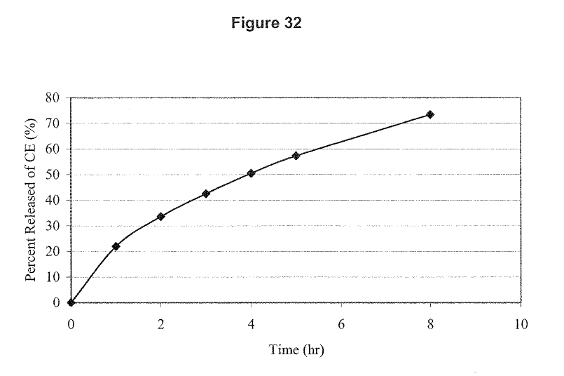

In some embodiments of the previous two embodiments:
the estrogen comprises conjugated estrogens;
the therapeutic agent comprises medroxyprogesterone acetate;
the dissolution profile of the estrogen from the tablet is substantially as shown in FIG. 32; and
the dissolution profile of the therapeutic agent from the tablet under type I therapeutic agent dissolution conditions is substantially as shown in FIG. 17.

In some embodiments:
the hydrophilic gel-forming polymer component of the first layer comprises from about 5% to about 15% by weight of the first layer; and
the hydrophilic gel-forming polymer component of the second layer comprises from about 15% to about 30% by weight of the second layer.

In some embodiments:

(a) the first layer further comprises:
a filler/diluent component comprising from about 50% to about 85% by weight of the first layer;
a filler/binder component comprising from about 5% to about 25% by weight of the first layer; and
optionally, a lubricant component comprising from about 0.01% to about 2% by weight of the first layer; and

(b) the second layer further comprises:
a filler/diluent component comprising from about 20% to about 60% by weight of the second layer;
a filler/binder component comprising from about 20% to about 60% by weight of the second layer;
optionally, a lubricant component comprising from about 0.01% to about 2% by weight of the second layer;
optionally, a disintegrant comprising up to about 4% by weight of the second layer; and
optionally, an antioxidant component comprising from about 0.01% to about 4% by weight of the second layer.

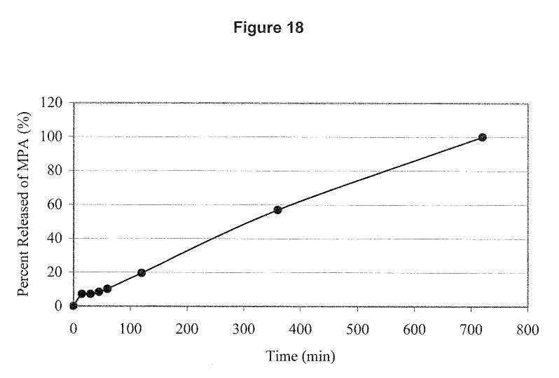

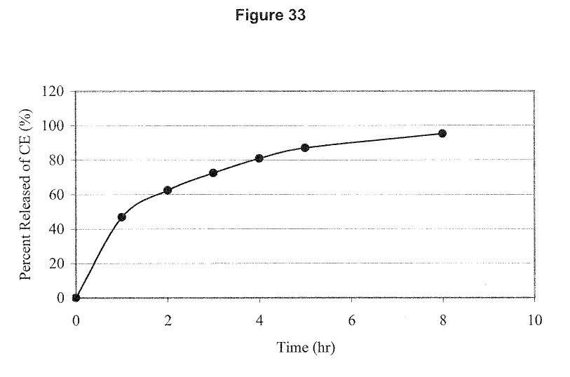

In some embodiments of the previous two embodiments:
the estrogen comprises conjugated estrogens;
the therapeutic agent comprises medroxyprogesterone acetate;
the dissolution profile of the estrogen from the tablet is substantially as shown in FIG. 33; and
the dissolution profile of the therapeutic agent from the tablet under type I therapeutic agent dissolution conditions is substantially as shown in FIG. 18.

In some embodiments:
the hydrophilic gel-forming polymer component of the first layer comprises from about 30% to about 40% by weight of the first layer; and
the hydrophilic gel-forming polymer component of the second layer comprises from about 1% to about 8% by weight of the second layer.

In some embodiments:

(a) the first layer further comprises:
a filler/diluent component comprising from about 30% to about 70% by weight of the first layer;
a filler/binder component comprising from about 5% to about 25% by weight of the first layer; and
optionally, a lubricant component comprising from about 0.01% to about 2% by weight of the first layer; and

(b) the second layer further comprises:
a filler/diluent component comprising from about 35% to about 75% by weight of the second layer;
a filler/binder component comprising from about 20% to about 60% by weight of the second layer;
optionally, a lubricant component comprising from about 0.01% to about 2% by weight of the second layer;
optionally, a disintegrant comprising up to about 4% by weight of the second layer; and
optionally, an antioxidant component comprising from about 0.01% to about 4% by weight of the second layer.

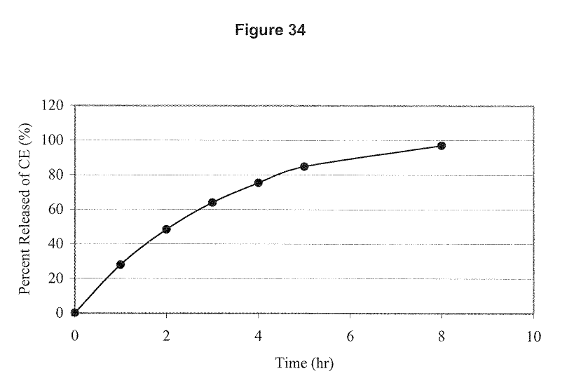

In some embodiments of the previous two embodiments:
the estrogen comprises conjugated estrogens;
the therapeutic agent comprises medroxyprogesterone acetate;
the dissolution profile of the estrogen from the tablet is substantially as shown in FIG. 34; and
the dissolution profile of the therapeutic agent from the tablet under type I therapeutic agent dissolution conditions is substantially as shown in FIG. 19.

In some embodiments:
the hydrophilic gel-forming polymer component of the first layer comprises from about 5% to about 15% by weight of the first layer; and
the hydrophilic gel-forming polymer component of the second layer comprises from about 1% to about 8% by weight of the second layer.

In some embodiments:

(a) the first layer further comprises:
a filler/diluent component comprising from about 50% to about 85% by weight of the first layer;
a filler/binder component comprising from about 5% to about 25% by weight of the first layer; and
optionally, a lubricant component comprising from about 0.01% to about 2% by weight of the first layer; and

(b) the second layer further comprises:
a filler/diluent component comprising from about 35% to about 75% by weight of the second layer;
a filler/binder component comprising from about 20% to about 60% by weight of the second layer;
optionally, a lubricant component comprising from about 0.01% to about 2% by weight of the second layer;
optionally, a disintegrant comprising up to about 4% by weight of the second layer; and
optionally, an antioxidant component comprising from about 0.01% to about 4% by weight of the second layer.

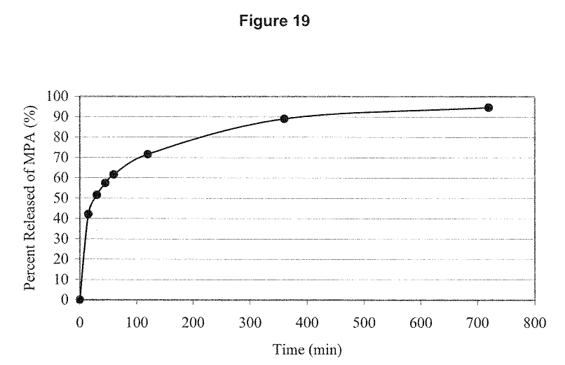

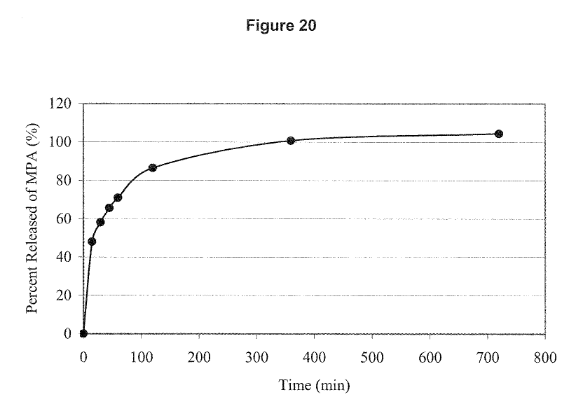

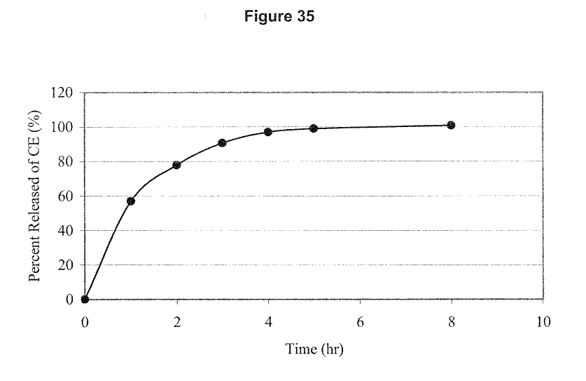

In some embodiments of the previous two embodiments:
the estrogen comprises conjugated estrogens;
the therapeutic agent comprises medroxyprogesterone acetate;
the dissolution profile of the estrogen from the tablet is substantially as shown in FIG. 35; and
the dissolution profile of the therapeutic agent from the tablet under type I therapeutic agent dissolution conditions is substantially as shown in FIG. 20.

In some embodiments:
the hydrophilic gel-forming polymer component of the first layer comprises from about 30% to about 40% by weight of the first layer; and
the hydrophilic gel-forming polymer component of the second layer comprises from about 8% to about 15% by weight of the second layer.

In some embodiments:

(a) the first layer further comprises:
a filler/diluent component comprising from about 30% to about 70% by weight of the first layer;
a filler/binder component comprising from about 5% to about 25% by weight of the first layer; and
optionally, a lubricant component comprising from about 0.01% to about 2% by weight of the first layer; and

(b) the second layer further comprises:
a filler/diluent component comprising from about 35% to about 70% by weight of the second layer;
a filler/binder component comprising from about 20% to about 60% by weight of the second layer;
optionally, a lubricant component comprising from about 0.01% to about 2% by weight of the second layer;
optionally, a disintegrant comprising up to about 4% by weight of the second layer; and
optionally, an antioxidant component comprising from about 0.01% to about 4% by weight of the second layer.

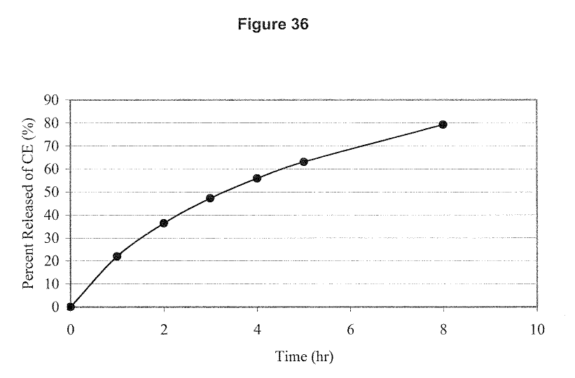

In some embodiments of the previous two embodiments:
the estrogen comprises conjugated estrogens;
the therapeutic agent comprises medroxyprogesterone acetate;
the dissolution profile of the estrogen from the tablet is substantially as shown in FIG. 36; and
the dissolution profile of the therapeutic agent from the tablet under type I therapeutic agent dissolution conditions is substantially as shown in FIG. 21.

In some embodiments:
the hydrophilic gel-forming polymer component of the first layer comprises from about 40% to about 60% by weight of the first layer; and
the hydrophilic gel-forming polymer component of the second layer comprises from about 1% to about 8% by weight of the second layer.

In some embodiments:

(a) the first layer further comprises:
a filler/diluent component comprising from about 10% to about 50% by weight of the first layer;
a filler/binder component comprising from about 5% to about 25% by weight of the first layer; and
optionally, a lubricant component comprising from about 0.01% to about 2% by weight of the first layer; and

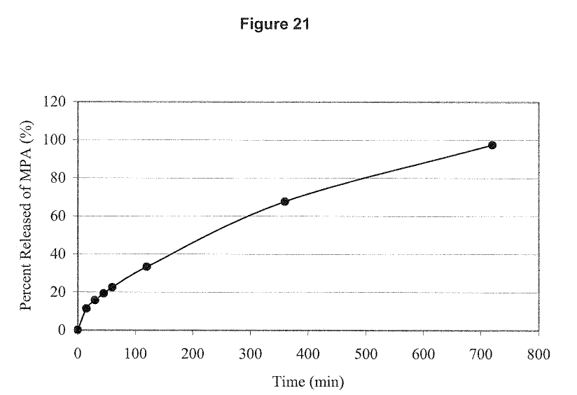

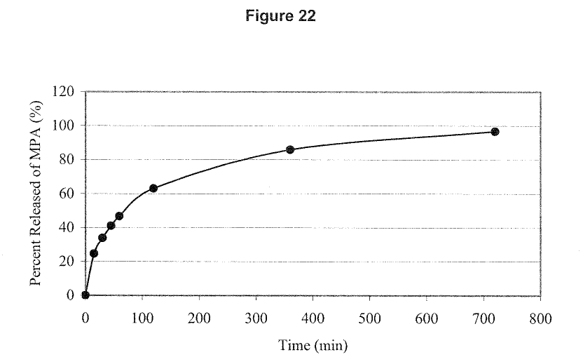

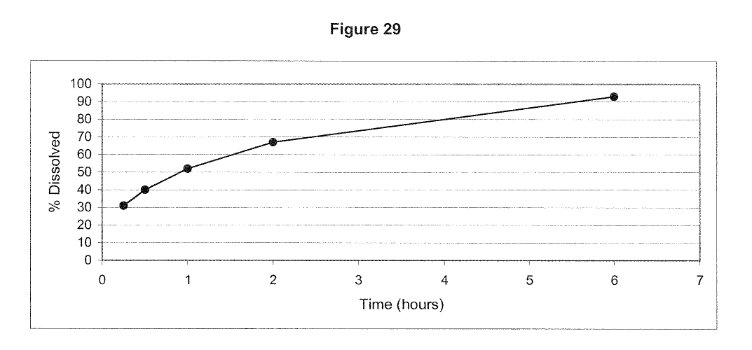

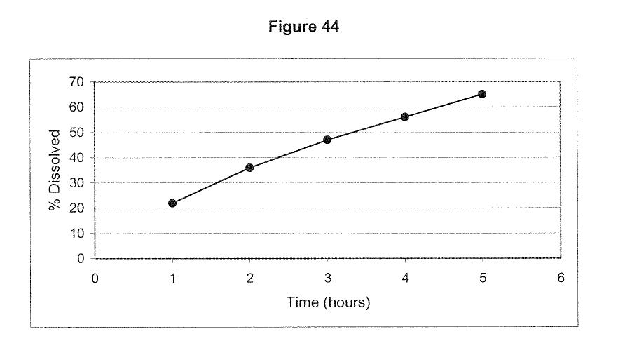

(b) the second layer further comprises:
a filler/diluent component comprising from about 35% to about 75% by weight of the second layer;
a filler/binder component comprising from about 20% to about 60% by weight of the second layer;
optionally, a lubricant component comprising from about 0.01% to about 2% by weight of the second layer;
optionally, a disintegrant comprising up to about 4% by weight of the second layer; and
optionally, an antioxidant component comprising from about 0.01% to about 4% by weight of the second layer.
   In some embodiments of the previous two embodiments:
the estrogen comprises conjugated estrogens;
the therapeutic agent comprises medroxyprogesterone acetate;
the dissolution profile of the estrogen from the tablet is substantially as shown in FIG. 37 or 44; and
the dissolution profile of the therapeutic agent from the tablet under type I therapeutic agent dissolution conditions is substantially as shown in FIG. 22 or 29.

In some embodiments:
the hydrophilic gel-forming polymer component of the first layer comprises from about 40% to about 60% by weight of the first layer; and
the hydrophilic gel-forming polymer component of the second layer comprises from about 8% to about 15% by weight of the second layer.

In some embodiments:

(a) the first layer further comprises:
a filler/diluent component comprising from about 10% to about 50% by weight of the first layer;
a filler/binder component comprising from about 5% to about 25% by weight of the first layer; and
optionally, a lubricant component comprising from about 0.01% to about 2% by weight of the first layer; and

(b) the second layer further comprises:
a filler/diluent component comprising from about 35% to about 70% by weight of the second layer;
a filler/binder component comprising from about 20% to about 60% by weight of the second layer;
optionally, a lubricant component comprising from about 0.01% to about 2% by weight of the second layer;
optionally, a disintegrant comprising up to about 4% by weight of the second layer; and
optionally, an antioxidant component comprising from about 0.01% to about 4% by weight of the second layer.

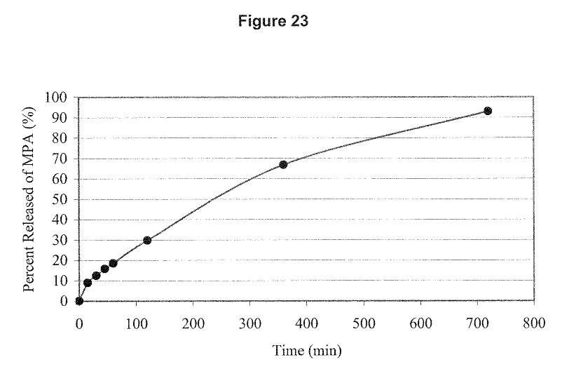

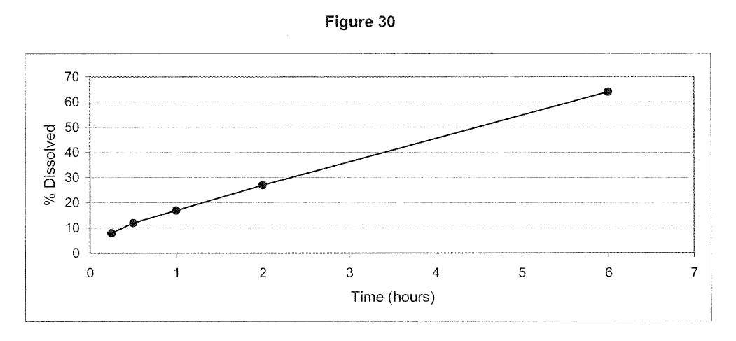

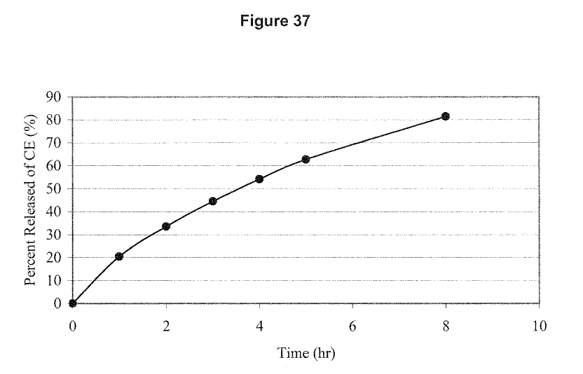

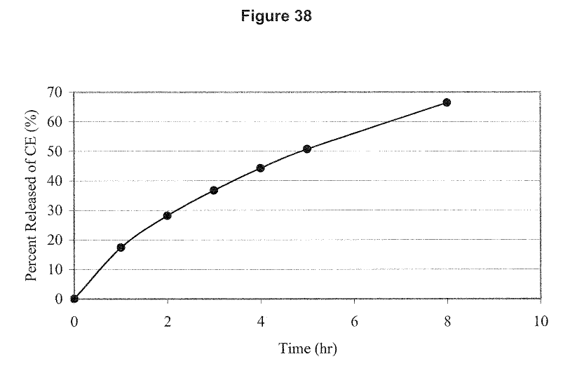

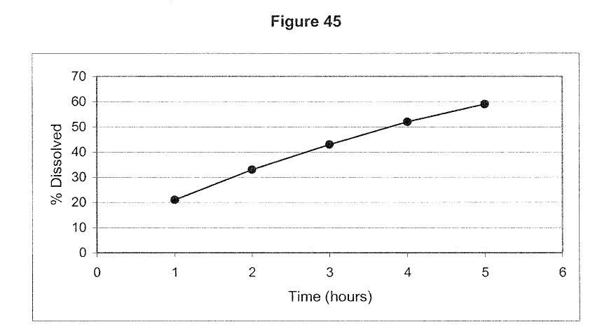

In some embodiments of the previous two embodiments:
the estrogen comprises conjugated estrogens;
the therapeutic agent comprises medroxyprogesterone acetate;
the dissolution profile of the estrogen from the tablet is substantially as shown in FIG. 38 or 45; and
the dissolution profile of the therapeutic agent from the tablet under type I therapeutic agent dissolution conditions is substantially as shown in FIG. 23 or 30.

In some embodiments:
the hydrophilic gel-forming polymer component of the first layer comprises from about 40% to about 60% by weight of the first layer; and
the hydrophilic gel-forming polymer component of the second layer comprises from about 15% to about 30% by weight of the second layer.

In some embodiments:

(a) the first layer further comprises:
a filler/diluent component comprising from about 10% to about 50% by weight of the first layer;
a filler/binder component comprising from about 5% to about 25% by weight of the first layer; and
optionally, a lubricant component comprising from about 0.01% to about 2% by weight of the first layer; and

(b) the second layer further comprises:
a filler/diluent component comprising from about 20% to about 60% by weight of the second layer;
a filler/binder component comprising from about 20% to about 60% by weight of the second layer;
optionally, a lubricant component comprising from about 0.01% to about 2% by weight of the second layer;
optionally, a disintegrant comprising up to about 4% by weight of the second layer; and
optionally, an antioxidant component comprising from about 0.01% to about 4% by weight of the second layer.

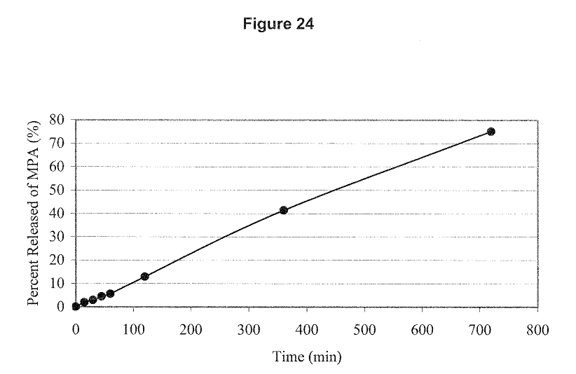

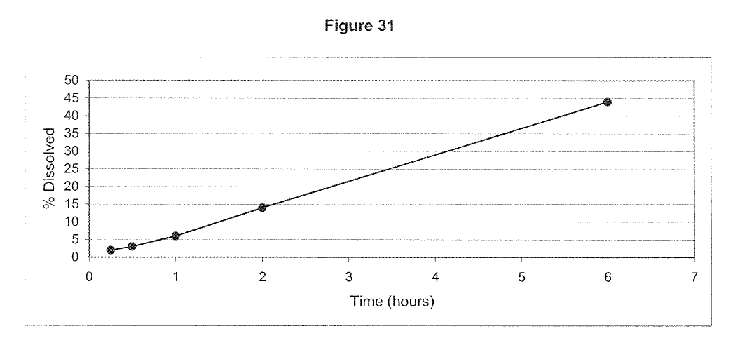

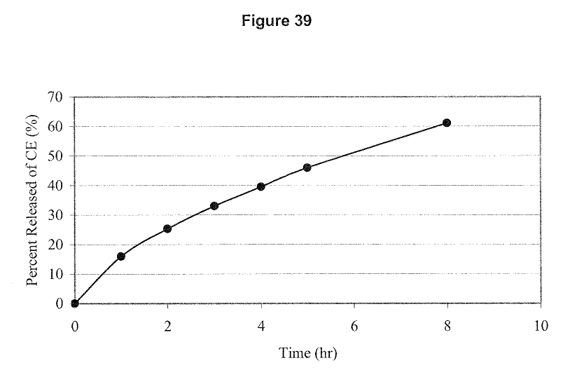

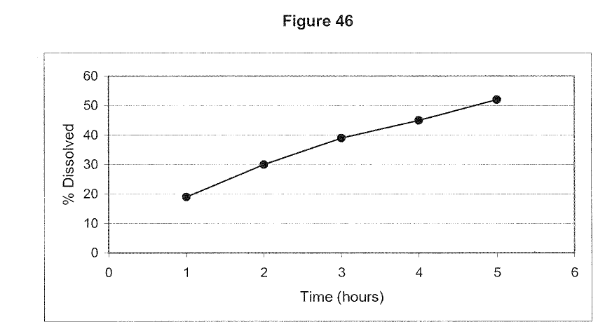

In some embodiments of the previous two embodiments:
the estrogen comprises conjugated estrogens;
the therapeutic agent comprises medroxyprogesterone acetate;
the dissolution profile of the estrogen from the tablet is substantially as shown in FIG. 39 or 46; and
the dissolution profile of the therapeutic agent from the tablet under type I therapeutic agent dissolution conditions is substantially as shown in FIG. 24 or 31.

In some embodiments:
the hydrophilic gel-forming polymer component of the first layer comprises from about 5% to about 15% by weight of the first layer; and
the hydrophilic gel-forming polymer component of the second layer comprises from about 8% to about 15% by weight of the second layer.

In some embodiments:

(a) the first layer further comprises:
a filler/diluent component comprising from about 50% to about 85% by weight of the first layer;
a filler/binder component comprising from about 5% to about 25% by weight of the first layer; and
optionally, a lubricant component comprising from about 0.01% to about 2% by weight of the first layer; and

(b) the second layer further comprises:
a filler/diluent component comprising from about 35% to about 70% by weight of the second layer;
a filler/binder component comprising from about 20% to about 60% by weight of the second layer;
optionally, a lubricant component comprising from about 0.01% to about 2% by weight of the second layer;
optionally, a disintegrant comprising up to about 4% by weight of the second layer; and
optionally, an antioxidant component comprising from about 0.01% to about 4% by weight of the second layer.

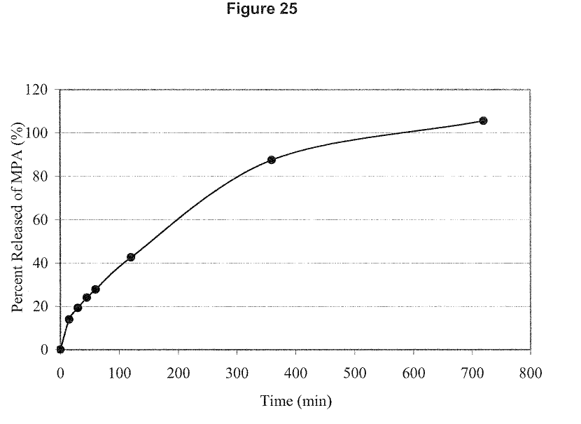

In some embodiments of the previous two embodiments:
the estrogen comprises conjugated estrogens;
the therapeutic agent comprises medroxyprogesterone acetate;
the dissolution profile of the estrogen from the tablet is substantially as shown in FIG. 40; and
the dissolution profile of the therapeutic agent from the tablet under type I therapeutic agent dissolution conditions is substantially as shown in FIG. 25.

In some embodiments:
the hydrophilic gel-forming polymer component of the first layer comprises from about 15% to about 30% by weight of the first layer; and
the hydrophilic gel-forming polymer component of the second layer comprises from about 15% to about 30% by weight of the second layer.

In some embodiments:

(a) the first layer further comprises:
a filler/diluent component comprising from about 40% to about 80% by weight of the first layer;
a filler/binder component comprising from about 5% to about 25% by weight of the first layer; and
optionally, a lubricant component comprising from about 0.01% to about 2% by weight of the first layer; and

(b) the second layer further comprises:
a filler/diluent component comprising from about 0.0% to about 60% (e.g., about 0.5% to about 60% or about 10% to about 60%) by weight of the second layer;
a filler/binder component comprising from about 20% to about 60% by weight of the second layer; and
optionally, a lubricant component comprising from about 0.01% to about 2% by weight of the second layer;
optionally, a disintegrant comprising up to about 4% by weight of the second layer; and
optionally, an antioxidant component comprising from about 0.01% to about 4% by weight of the second layer.

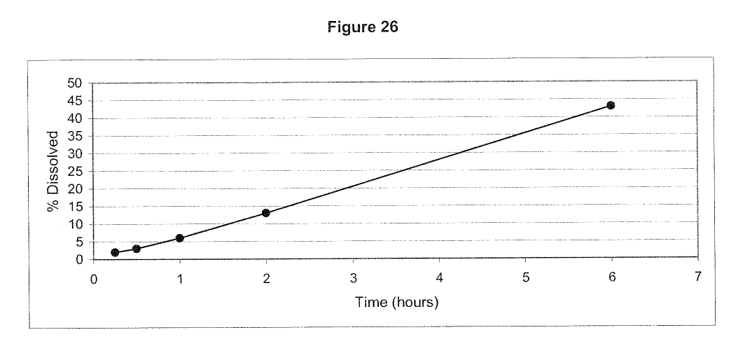

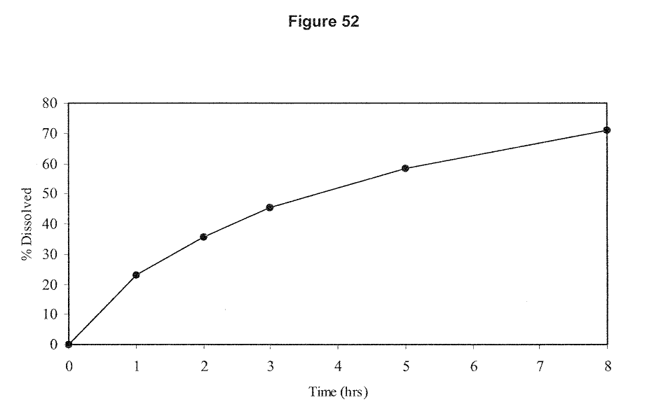

In some embodiments of the previous two embodiments:
the estrogen comprises conjugated estrogens;
the therapeutic agent comprises medroxyprogesterone acetate;
the dissolution profile of the estrogen from the tablet is substantially as shown in FIG. 41; and
the dissolution profile of the therapeutic agent from the tablet under type I therapeutic agent dissolution conditions is substantially as shown in FIG. 26; or
the estrogen comprises conjugated estrogens;
the therapeutic agent comprises bazedoxifene acetate;
the dissolution profile of the estrogen from the tablet is substantially as shown in FIG. 52; and
the dissolution profile of the therapeutic agent from the tablet under type II therapeutic agent dissolution conditions is substantially as shown in FIG. 49.

In some embodiments:
the hydrophilic gel-forming polymer component of the first layer comprises from about 15% to about 30% by weight of the first layer; and
the hydrophilic gel-forming polymer component of the second layer comprises from about 1% to about 8% by weight of the second layer.

In some embodiments:

(a) the first layer further comprises:
a filler/diluent component comprising from about 40% to about 80% by weight of the first layer;
a filler/binder component comprising from about 5% to about 25% by weight of the first layer; and
optionally, a lubricant component comprising from about 0.01% to about 2% by weight of the first layer; and

(b) the second layer further comprises:
a filler/diluent component comprising from about 35% to about 75% by weight of the second layer;
a filler/binder component comprising from about 20% to about 60% by weight of the second layer;
optionally, a lubricant component comprising from about 0.01% to about 2% by weight of the second layer;
optionally, a disintegrant comprising up to about 4% by weight of the second layer; and
optionally, an antioxidant component comprising from about 0.01% to about 4% by weight of the second layer.

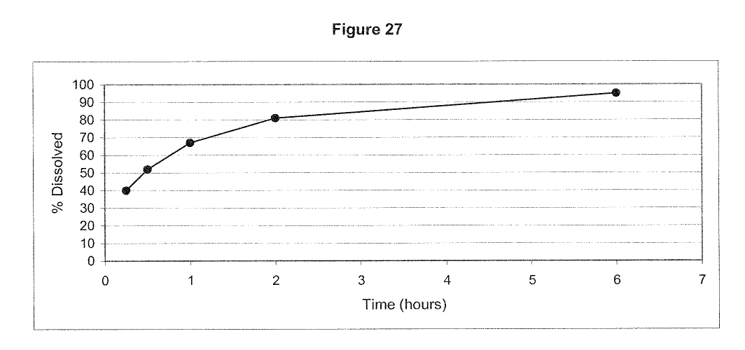

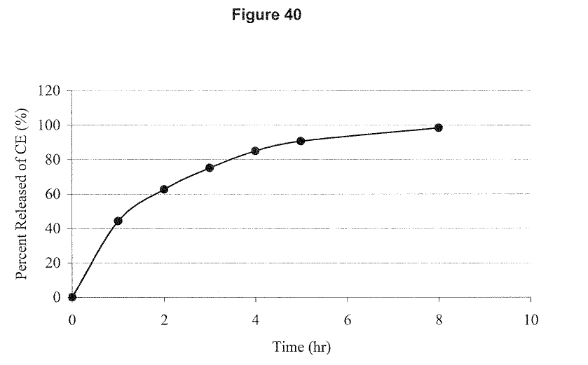

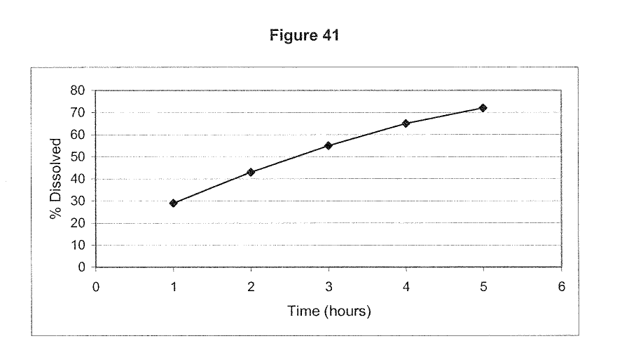

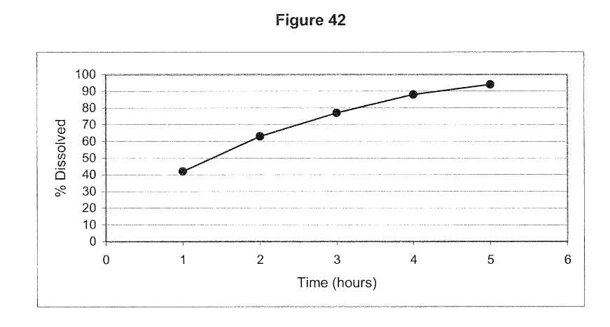

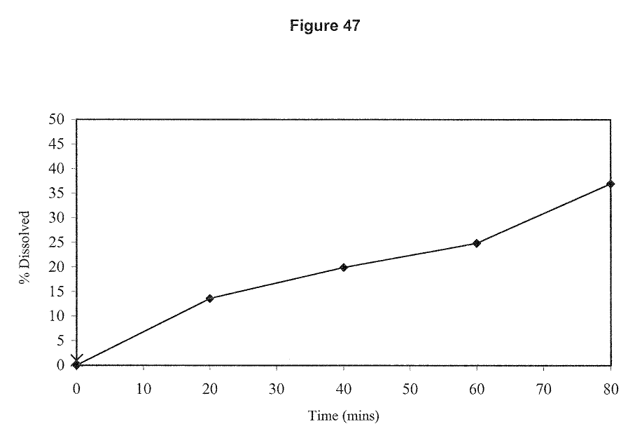

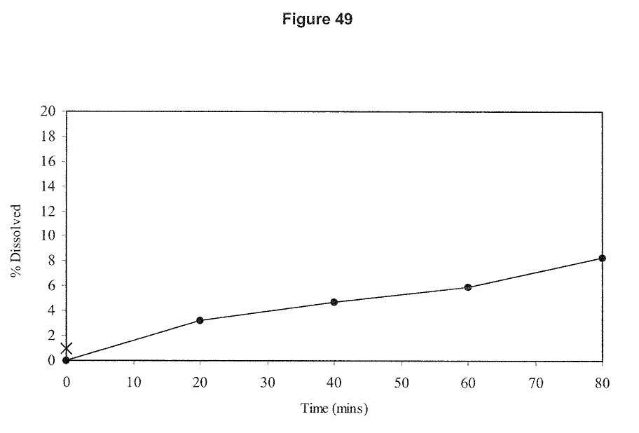

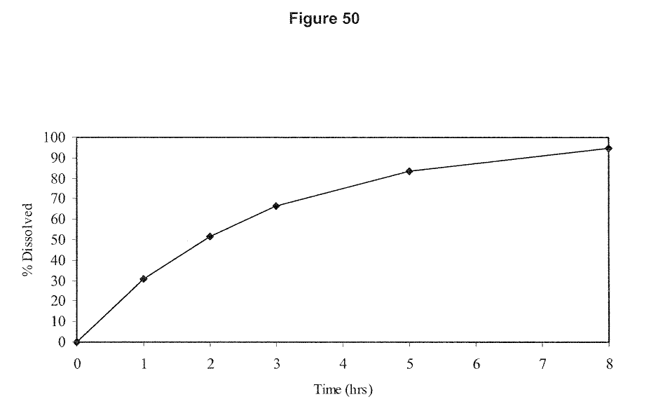

In some embodiments of the previous two embodiments:
the estrogen comprises conjugated estrogens;
the therapeutic agent comprises medroxyprogesterone acetate;
the dissolution profile of the estrogen from the tablet is substantially as shown in FIG. 42; and
the dissolution profile of the therapeutic agent from the tablet under type I therapeutic agent dissolution conditions is substantially as shown in FIG. 27; or
the estrogen comprises conjugated estrogens;
the therapeutic agent comprises bazedoxifene acetate;
the dissolution profile of the estrogen from the tablet is substantially as shown in FIG. 50; and
the dissolution profile of the therapeutic agent from the tablet under type II therapeutic agent dissolution conditions is substantially as shown in FIG. 47.

In some embodiments:
the hydrophilic gel-forming polymer component of the first layer comprises from about 15% to about 30% by weight of the first layer; and
the hydrophilic gel-forming polymer component of the second layer comprises from about 8% to about 15% by weight of the second layer.

In some embodiments:

(a) the first layer further comprises:
a filler/diluent component comprising from about 40% to about 80% by weight of the first layer;
a filler/binder component comprising from about 5% to about 25% by weight of the first layer; and
optionally, a lubricant component comprising from about 0.01% to about 2% by weight of the first layer; and

(b) the second layer further comprises:
a filler/diluent component comprising from about 35% to about 70% by weight of the second layer;
a filler/binder component comprising from about 20% to about 60% by weight of the second layer;
optionally, a lubricant component comprising from about 0.01% to about 2% by weight of the second layer;
optionally, a disintegrant comprising up to about 4% by weight of the second layer; and
optionally, an antioxidant component comprising from about 0.01% to about 4% by weight of the second layer.

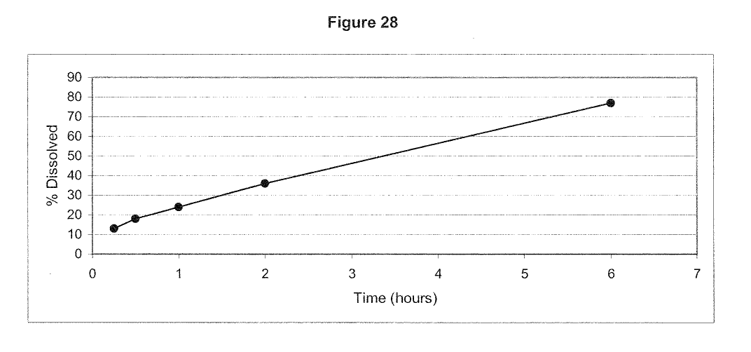

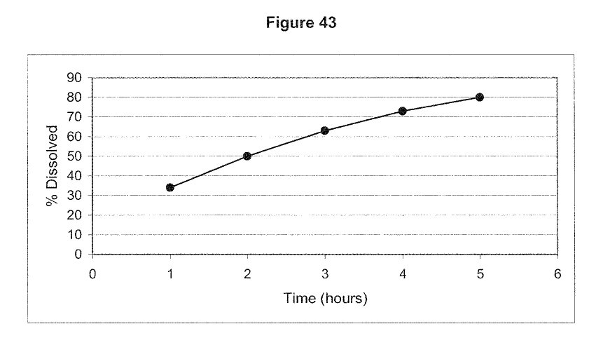

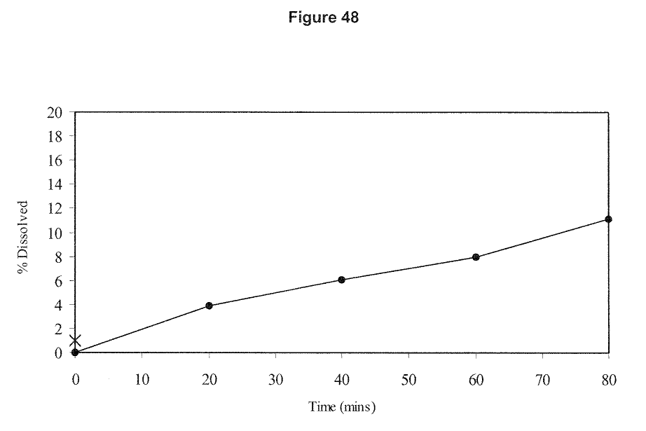

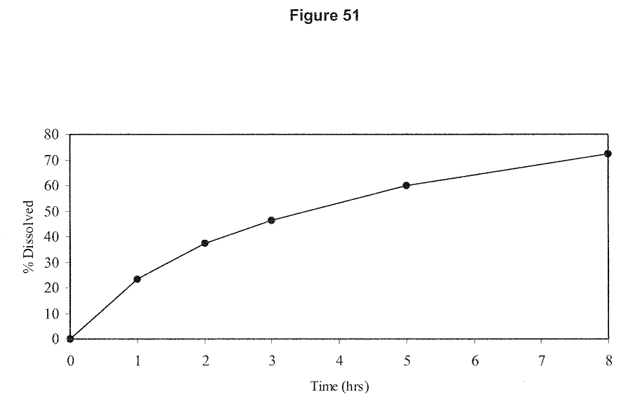

In some embodiments of the previous two embodiments:
the estrogen comprises conjugated estrogens;
the therapeutic agent comprises medroxyprogesterone acetate;
the dissolution profile of the estrogen from the tablet is substantially as shown in FIG. 43; and
the dissolution profile of the therapeutic agent from the tablet under type I therapeutic agent dissolution conditions is substantially as shown in FIG. 28; or
the estrogen comprises conjugated estrogens;
the therapeutic agent comprises bazedoxifene acetate;
the dissolution profile of the estrogen from the tablet is substantially as shown in FIG. 51; and
the dissolution profile of the therapeutic agent from the tablet under type II therapeutic agent dissolution conditions is substantially as shown in FIG. 48.

In some embodiments:
the hydrophilic gel-forming polymer component of the first layer comprises from about 50% to about 60% by weight of the first layer; and
the hydrophilic gel-forming polymer component of the second layer comprises from about 1% to about 8% by weight of the second layer.

In some embodiments:
the hydrophilic gel-forming polymer component of the first layer comprises from about 50% to about 60% by weight of the first layer; and
the hydrophilic gel-forming polymer component of the second layer comprises from about 8% to about 15% by weight of the second layer.

In some embodiments:
the hydrophilic gel-forming polymer component of the first layer comprises from about 50% to about 60% by weight of the first layer; and
the hydrophilic gel-forming polymer component of the second layer comprises from about 15% to about 30% by weight of the second layer.

In some embodiments:
the hydrophilic gel-forming polymer component of the first layer comprises from about 22% to about 33% by weight of the first layer; and
the hydrophilic gel-forming polymer component of the second layer comprises from about 15% to about 25% by weight of the second layer.

In some embodiments:
the hydrophilic gel-forming polymer component of the first layer comprises from about 22% to about 33% by weight of the first layer;
the hydrophilic gel-forming polymer component of the second layer comprises from about 15% to about 25% by weight of the second layer.

In some embodiments:
the hydrophilic gel-forming polymer component of the first layer comprises from about 22% to about 33% by weight of the first layer; and
the hydrophilic gel-forming polymer component of the second layer comprises from about 8% to about 15% by weight of the second layer.

In some embodiments:
the hydrophilic gel-forming polymer component of the first layer comprises from about 22% to about 33% by weight of the first layer; and
the hydrophilic gel-forming polymer component of the second layer comprises from about 2% to about 7% by weight of the second layer.

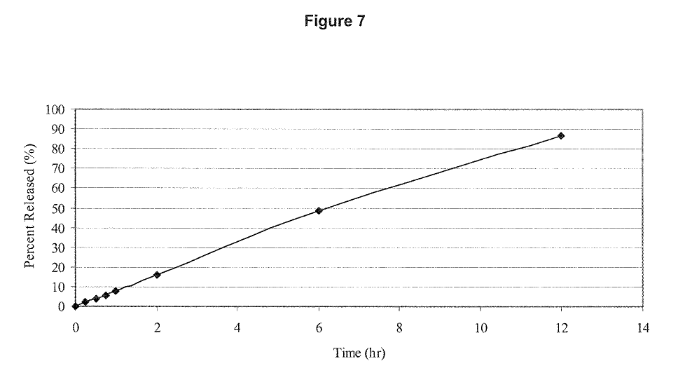

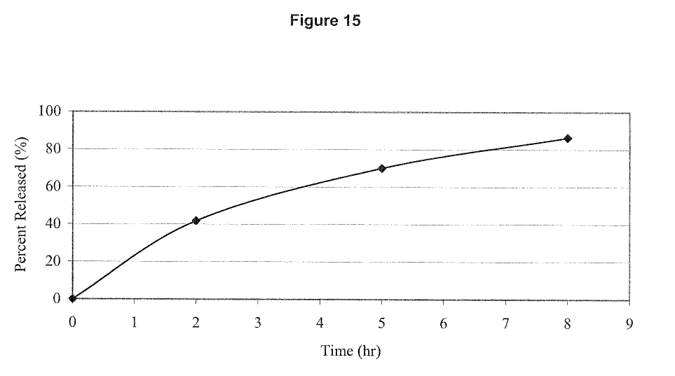

In some embodiments:
the hydrophilic gel-forming polymer component of the first layer comprises from about 22% to about 33% by weight of the first layer; and
the hydrophilic gel-forming polymer component of the second layer comprises from about 5% to about 15% of a first hydroxypropylmethylcellulose polymer by weight of the second layer and from about 5% to about 15% of a second hydroxypropylmethylcellulose polymer by weight of the second layer;
the first hydroxypropylmethylcellulose polymer has an apparent viscosity from about 3000 to about 6000 cP;
the second hydroxypropylmethylcellulose polymer has an apparent viscosity from about 80,000 to about 100,000 cP;
the estrogen comprises conjugated estrogens;
the therapeutic agent comprises medroxyprogesterone acetate;
the dissolution profile of the estrogen from the tablet is substantially as shown in FIG. 15; and
the dissolution profile of the therapeutic agent from the tablet under type I therapeutic agent dissolution conditions is substantially as shown in FIG. 7.

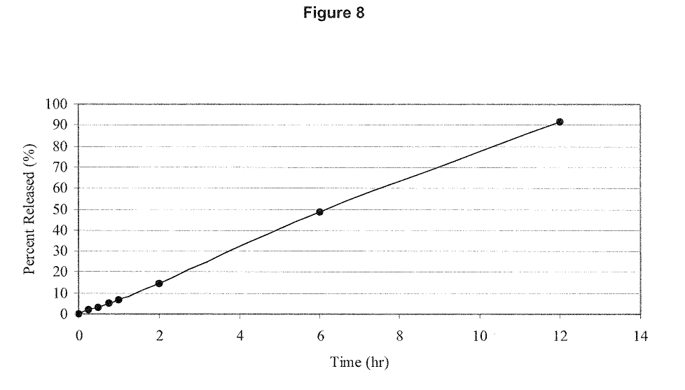

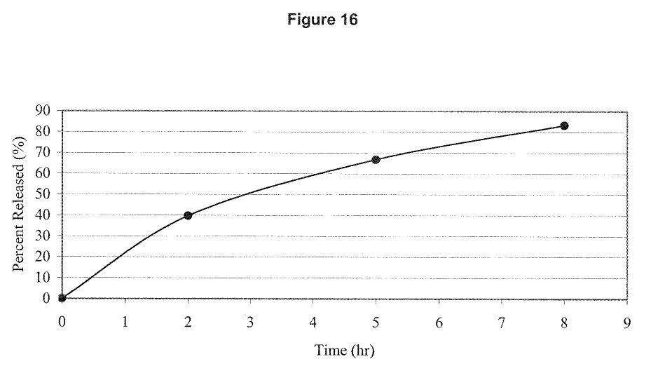

In some embodiments:
the hydrophilic gel-forming polymer component of the first layer comprises from about 22% to about 33% by weight of the first layer; and
the hydrophilic gel-forming polymer component of the second layer comprises from about 5% to about 15% of a first hydroxypropylmethylcellulose polymer by weight of the second layer and from about 5% to about 15% of a second hydroxypropylmethylcellulose polymer by weight of the second layer;
the first hydroxypropylmethylcellulose polymer has an apparent viscosity from about 80 to about 120 cP;
the second hydroxypropylmethylcellulose polymer has an apparent viscosity from about 80,000 to about 100,000 cP;
the estrogen comprises conjugated estrogens;
the therapeutic agent comprises medroxyprogesterone acetate;
the dissolution profile of the estrogen from the tablet is substantially as shown in FIG. 16; and
the dissolution profile of the therapeutic agent from the tablet under type I therapeutic agent dissolution conditions is substantially as shown in FIG. 8.

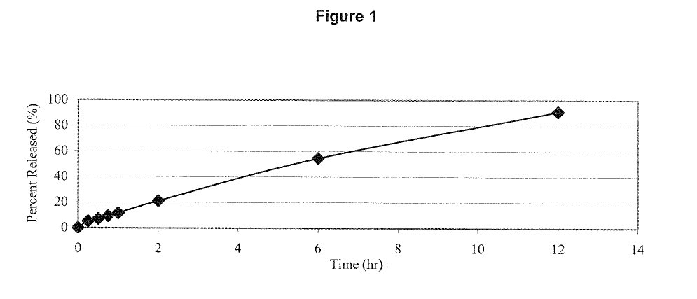

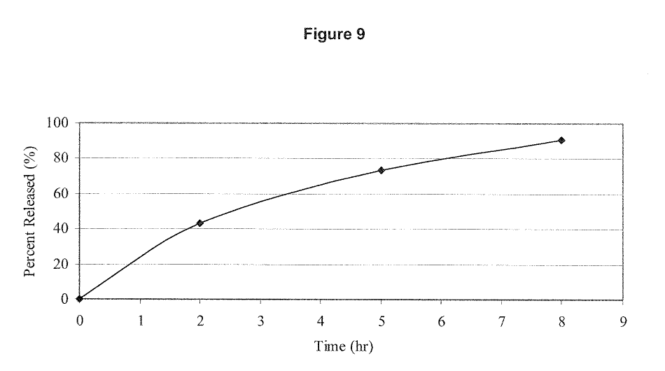

In some embodiments:
the hydrophilic gel-forming polymer component of the first layer comprises from about 22% to about 33% by weight of the first layer; and
the hydrophilic gel-forming polymer component of the second layer comprises from about 15% to about 25% by weight of the second layer;
the estrogen comprises conjugated estrogens;
the therapeutic agent comprises medroxyprogesterone acetate;
the dissolution profile of the estrogen from the tablet is substantially as shown in FIG. 9; and
the dissolution profile of the therapeutic agent from the tablet under type I therapeutic agent dissolution conditions is substantially as shown in FIG. 1.

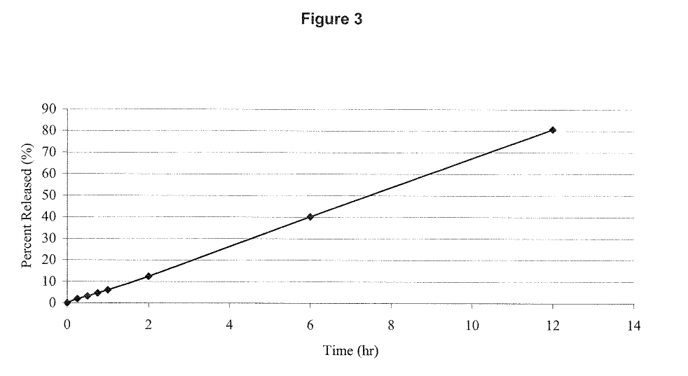

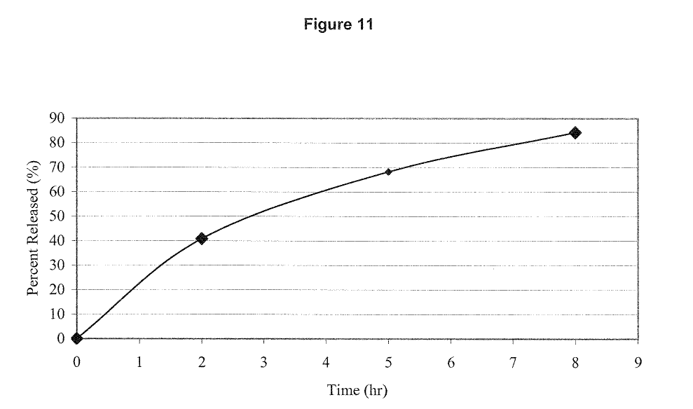

In some embodiments:
the hydrophilic gel-forming polymer component of the first layer comprises from about 22% to about 33% by weight of the first layer; and the hydrophilic gel-forming polymer component of the second layer comprises from about 15% to about 25% by weight of the second layer;
the estrogen comprises conjugated estrogens;
the therapeutic agent comprises medroxyprogesterone acetate;
the dissolution profile of the estrogen from the tablet is substantially as shown in FIG. 11; and
the dissolution profile of the therapeutic agent from the tablet under type I therapeutic agent dissolution conditions is substantially as shown in FIG. 3.

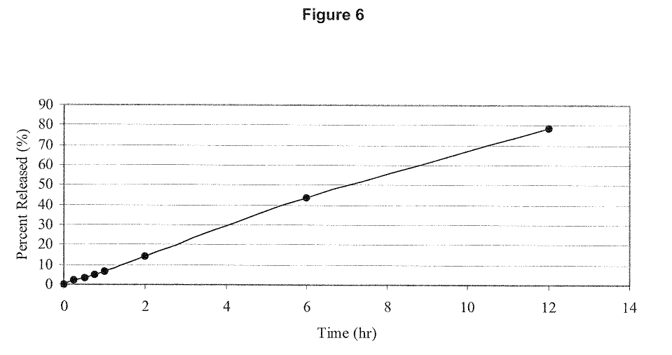

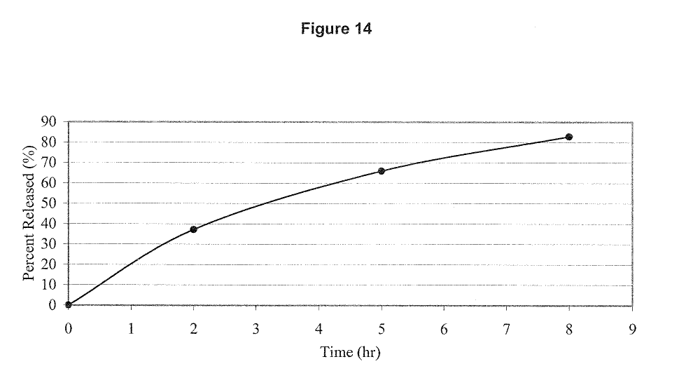

In some embodiments:
the hydrophilic gel-forming polymer component of the first layer comprises from about 22% to about 33% by weight of the first layer; and
the hydrophilic gel-forming polymer component of the second layer comprises from about 15% to about 25% by weight of the second layer;
the estrogen comprises conjugated estrogens;
the therapeutic agent comprises medroxyprogesterone acetate;
the dissolution profile of the estrogen from the tablet is substantially as shown in FIG. 14; and
the dissolution profile of the therapeutic agent from the tablet under type I therapeutic agent dissolution conditions is substantially as shown in FIG. 6.

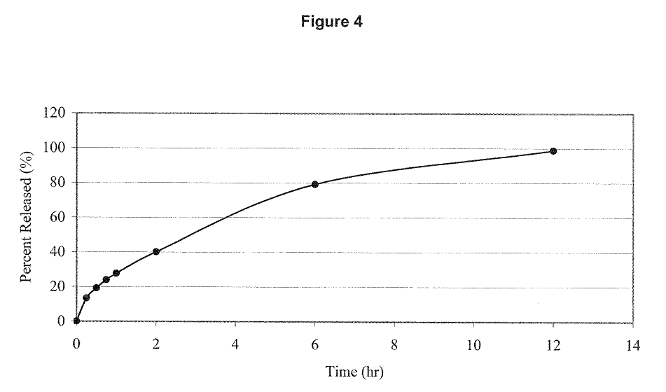

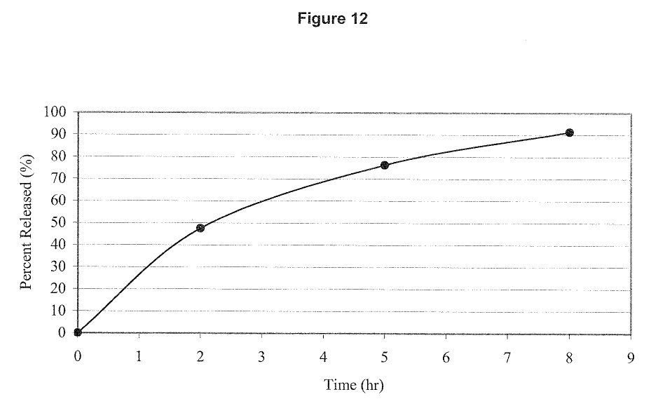

In some embodiments:
the hydrophilic gel-forming polymer component of the first layer comprises from about 22% to about 33% by weight of the first layer; and
the hydrophilic gel-forming polymer component of the second layer comprises from about 5% to about 15% by weight of the second layer;
the estrogen comprises conjugated estrogens;
the therapeutic agent comprises medroxyprogesterone acetate;
the dissolution profile of the estrogen from the tablet is substantially as shown in FIG. 12; and
the dissolution profile of the therapeutic agent from the tablet under type I therapeutic agent dissolution conditions is substantially as shown in FIG. 4.

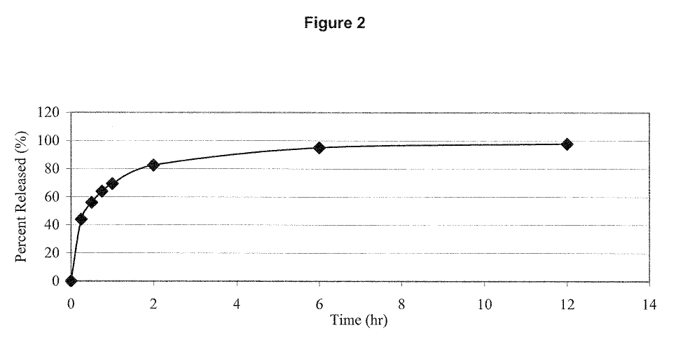

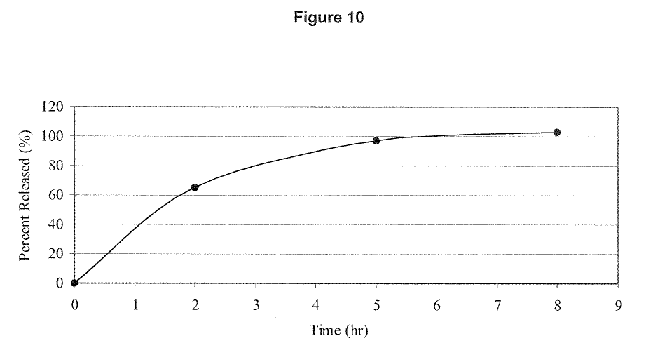

In some embodiments:
the hydrophilic gel-forming polymer component of the first layer comprises from about 22% to about 33% by weight of the first layer; and the hydrophilic gel-forming polymer component of the second layer comprises from about 1% to about 10% by weight of the second layer;
the estrogen comprises conjugated estrogens;
the therapeutic agent comprises medroxyprogesterone acetate;
the dissolution profile of the estrogen from the tablet is substantially as shown in FIG. 10; and
the dissolution profile of the therapeutic agent from the tablet under type I therapeutic agent dissolution conditions is substantially as shown in FIG. 2.

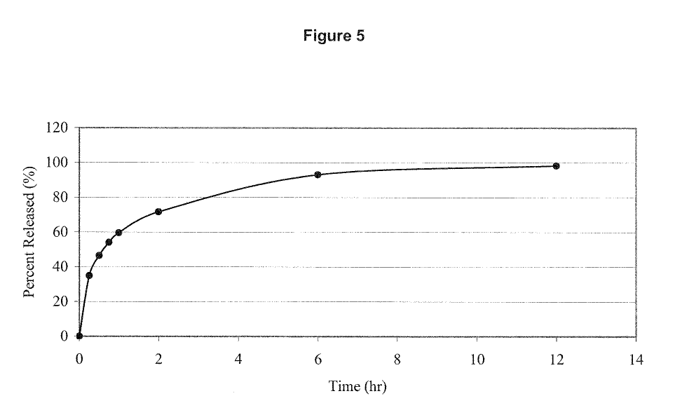

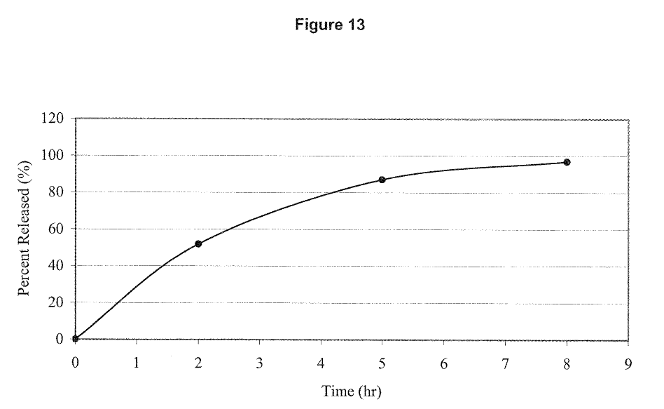

In some embodiments:
the hydrophilic gel-forming polymer component of the first layer comprises from about 22% to about 33% by weight of the first layer; and
the hydrophilic gel-forming polymer component of the second layer comprises from about 1% to about 10% by weight of the second layer;
the estrogen comprises conjugated estrogens;
the therapeutic agent comprises medroxyprogesterone acetate;
the dissolution profile of the estrogen from the tablet is substantially as shown in FIG. 13; and
the dissolution profile of the therapeutic agent from the tablet under type I therapeutic agent dissolution conditions is substantially as shown in FIG. 5.

In some embodiments:
the filler/diluent component of the first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the filler/binder component of the first layer comprises one or more of microcrystalline cellulose, polyvinylpyrrolidone, copovidone, polyvinylalcohol, starch, gelatin, gum arabic, gum acacia, and gum tragacanth;
the hydrophilic gel-forming polymer component of the first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional lubricant component of the first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the filler/diluent component of the second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the filler/binder component of the second layer comprises one or more of microcrystalline cellulose, polyvinylpyrrolidone, copovidone, polyvinylalcohol, starch, gelatin, gum arabic, gum acacia, and gum tragacanth;
the hydrophilic gel-forming polymer of the second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional lubricant component of the second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the optional disintegrant of the second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose; and
the optional antioxidant component of the second layer, if present, comprises one or more of ascorbic acid, sodium ascorbate, ascorbyl palmitate, vitamin E, vitamin E acetate, butylated hydroxytoluene, and butylated hydroxyanisole.

In some embodiments:
the filler/diluent component of the first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the filler/binder component of the first layer comprises one or more of microcrystalline cellulose, polyvinylpyrrolidone, copovidone, polyvinylalcohol, starch, gelatin, gum arabic, gum acacia, and gum tragacanth;
the hydrophilic gel-forming polymer component of the first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional lubricant component of the first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the filler/diluent component of the second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the filler/binder component of the second layer comprises one or more of microcrystalline cellulose, polyvinylpyrrolidone, copovidone, polyvinylalcohol, starch, gelatin, gum arabic, gum acacia, and gum tragacanth;
the hydrophilic gel-forming polymer of the second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional lubricant component of the second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the optional disintegrant of the second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
the optional antioxidant component of the second layer, if present, comprises one or more of ascorbic acid, sodium ascorbate, ascorbyl palmitate, vitamin E, vitamin E acetate, butylated hydroxytoluene, and butylated hydroxyanisole;
the estrogen comprises conjugated estrogens; and
the one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

In some embodiments:
the filler/diluent component of the first layer comprises one or more of lactose or lactose monohydrate;
the filler/binder component of the first layer comprises microcrystalline cellulose;
the hydrophilic gel-forming polymer component of the first layer comprises hydroxypropylmethylcellulose;
the optional lubricant component of the first layer, if present, comprises magnesium stearate;
the filler/diluent component of the second layer comprises one or more of lactose or lactose monohydrate;
the filler/binder component of the second layer comprises microcrystalline cellulose;
the hydrophilic gel-forming polymer component of the second layer comprises hydroxypropylmethylcellulose;
the optional lubricant component of the second layer, if present, comprises magnesium stearate;
the optional disintegrant of the second layer, if present, comprises croscarmellose sodium; and
the optional antioxidant component of the second layer, if present, comprises one or more of ascorbic acid and vitamin E acetate.

In some embodiments:
the filler/diluent component of the first layer comprises one or more of lactose or lactose monohydrate;
the filler/binder component of the first layer comprises microcrystalline cellulose;
the hydrophilic gel-forming polymer component of the first layer comprises hydroxypropylmethylcellulose;
the optional lubricant component of the first layer, if present, comprises magnesium stearate;
the filler/diluent component of the second layer comprises one or more of lactose or lactose monohydrate;
the filler/binder component of the second layer comprises microcrystalline cellulose;
the hydrophilic gel-forming polymer component of the second layer comprises hydroxypropylmethylcellulose;
the optional lubricant component of the second layer, if present, comprises magnesium stearate;
the optional disintegrant of the second layer, if present, comprises croscarmellose sodium;
the optional antioxidant component of the second layer, if present, comprises one or more of ascorbic acid and vitamin E acetate;
the estrogen comprises conjugated estrogens; and
the one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

In some embodiments, the present invention provides a tablet selected from a plurality of tablets of the invention, wherein the plurality has a mean dissolution profile wherein:

the mean of % of the estrogen released per tablet after 1, 2, 3, 4, and 5 hours under estrogen dissolution conditions is substantially equal to the sum of f, a*A, b*B, c*A², d*B², and e*A*B; and

the mean of % of the therapeutic agent per tablet released after 0.25, 0.5, 1, 2, and 6 hours under type I therapeutic agent dissolution conditions is substantially equal to the sum of m, n*A, o*B, p*A², q*B², and r*A*B;

wherein:

A is the % of hydrophilic gel-forming polymer by weight of the first layer;

B is the % of hydrophilic gel-forming polymer by weight of the second layer;

f, at 1 hour, is 84.405;

a, at 1 hour, is −1.801;

b, at 1 hour, is −3.141;

c, at 1 hour, is 0.0159;

d, at 1 hour, is 0.0991;

e, at 1 hour, is 0.00609;

f, at 2 hours, is 112.029;

a, at 2 hours, is −1.825;

b, at 2 hours, is −4.401;

c, at 2 hours, is 0.0134;

d, at 2 hours, is 0.131;

e, at 2 hours, is 0.00794;

f, at 3 hours, is 128.469;

a, at 3 hours, is −1.687;

b, at 3 hours, is −5.218;

c, at 3 hours, is 0.0105;

d, at 3 hours, is 0.153;

e, at 3 hours, is 0.00741;

f, at 4 hours, is 133.525;

a, at 4 hours, is −1.437;

b, at 4 hours, is −5.053;

c, at 4 hours, is 0.00776;

d, at 4 hours, is 0.152;

e, at 4 hours, is 0.000658;

f, at 5 hours, is 133.182;

a, at 5 hours, is −1.064;

b, at 5 hours, is −4.893;

c, at 5 hours, is 0.004363;

d, at 5 hours, is 0.1558;

e, at 5 hours, is −0.0076;

m, at 0.25 hour, is 94.7399;

n, at 0.25 hour, is −0.2561;

o, at 0.25 hour, is −10.7494;

p, at 0.25 hour, is −0.0038874;

q, at 0.25 hour, is 0.3088;

r, at 0.25 hour, is 0.02228;

m, at 0.5 hour, is 113.1339;

n, at 0.5 hour, is −0.2832;

o, at 0.5 hour, is −12.549;

p, at 0.5 hour, is −0.00428;

q, at 0.5 hour, is 0.35267;

r, at 0.5 hour, is 0.025698;

m, at 1 hour, is 133.966;

n, at 1 hour, is −0.446;

o, at 1 hour, is −14.0527;

p, at 1 hour, is −0.0021667;

q, at 1 hour, is 0.38816;

r, at 1 hour, is 0.02607;

m, at 2 hours, is 153.718;

n, at 2 hours, is −0.8427;

o, at 2 hours, is −14.196;

p, at 2 hours, is 0.003872;

q, at 2 hours, is 0.38144;

r, at 2 hours, is 0.023435;

m, at 6 hours, is 133.7326;

n, at 6 hours, is −1.134;

o, at 6 hours, is −4.458;

p, at 6 hours, is 0.0115;

q, at 6 hours, is 0.05789; and

r, at 6 hours, is 0.0006761.

In some embodiments, the present invention provides a tablet selected from a plurality of tablets of the invention, wherein the plurality has a mean dissolution profile wherein:

the mean of % of the estrogen released per tablet after 1, 2, 3, 4, and 5 hours under estrogen dissolution conditions is substantially equal to the sum of f, a*A, b*B, c*A², d*B², and e*A*B; and

A is the % of hydrophilic gel-forming polymer by weight of the first layer;

B is the % of hydrophilic gel-forming polymer by weight of the second layer;

f, at 1 hour, is 84.405;

a, at 1 hour, is −1.801;

b, at 1 hour, is −3.141;

c, at 1 hour, is 0.0159;

d, at 1 hour, is 0.0991;

e, at 1 hour, is 0.00609;

f, at 2 hours, is 112.029;

a, at 2 hours, is −1.825;

b, at 2 hours, is −4.401;

c, at 2 hours, is 0.0134;

d, at 2 hours, is 0.131;

e, at 2 hours, is 0.00794;

f, at 3 hours, is 128.469;

a, at 3 hours, is −1.687;

b, at 3 hours, is −5.218;

c, at 3 hours, is 0.0105;

d, at 3 hours, is 0.153;

e, at 3 hours, is 0.00741;

f, at 4 hours, is 133.525;

a, at 4 hours, is −1.437;

b, at 4 hours, is −5.053;

c, at 4 hours, is 0.00776;

d, at 4 hours, is 0.152;

e, at 4 hours, is 0.000658;

f, at 5 hours, is 133.182;

a, at 5 hours, is −1.064;

b, at 5 hours, is −4.893;

c, at 5 hours, is 0.004363;

d, at 5 hours, is 0.1558; and

e, at 5 hours, is −0.0076.

In some embodiments, the present invention provides a tablet selected from a plurality of tablets of the invention, wherein the plurality has a mean dissolution profile wherein:

the mean of % of the therapeutic agent per tablet released after 0.25, 0.5, 1, 2, and 6 hours under type I therapeutic agent dissolution conditions is substantially equal to the sum of m, n*A, o*B, p*A², q*B², and r*A*B; and

A is the % of hydrophilic gel-forming polymer by weight of the first layer;

B is the % of hydrophilic gel-forming polymer by weight of the second layer;

m, at 0.25 hour, is 94.7399;

n, at 0.25 hour, is −0.2561;

o, at 0.25 hour, is −10.7494;

p, at 0.25 hour, is −0.0038874;

q, at 0.25 hour, is 0.3088;

r, at 0.25 hour, is 0.02228;

m, at 0.5 hour, is 113.1339;

n, at 0.5 hour, is −0.2832;

o, at 0.5 hour, is −12.549;

p, at 0.5 hour, is −0.00428;

q, at 0.5 hour, is 0.35267;

r, at 0.5 hour, is 0.025698;

m, at 1 hour, is 133.966;

n, at 1 hour, is −0.446;

o, at 1 hour, is −14.0527;

p, at 1 hour, is −0.0021667;

q, at 1 hour, is 0.38816;

r, at 1 hour, is 0.02607;

m, at 2 hours, is 153.718;

n, at 2 hours, is −0.8427;

o, at 2 hours, is −14.196;

p, at 2 hours, is 0.003872;

q, at 2 hours, is 0.38144;

r, at 2 hours, is 0.023435;

m, at 6 hours, is 133.7326;

n, at 6 hours, is −1.134;

o, at 6 hours, is −4.458;

p, at 6 hours, is 0.0115;

q, at 6 hours, is 0.05789; and

r, at 6 hours, is 0.0006761.

In some embodiments:
the estrogen comprises conjugated estrogens; and
the dissolution profile of the estrogen from the tablet under estrogen dissolution conditions is substantially as shown in any one of FIGS. 9 to 16, 32 to 46, and 50 to 52.

In some embodiments:
the therapeutic agent comprises medroxyprogesterone acetate; and
the dissolution profile of the therapeutic agent from the tablet under type I therapeutic agent dissolution conditions is substantially as shown in any one of FIGS. 1 to 8 and 17 to 31.

In some embodiments:
the therapeutic agent comprises 1-[4-(2-azepan-1-yl-ethoxy)-benzyl]-2-(4-hydroxy-phenyl)-3-methyl-1H-indol-5-ol, acetic acid salt; and
the dissolution profile of the therapeutic agent from the tablet under type II therapeutic agent dissolution conditions is substantially as shown in any one of FIGS. 47 to 49.

In some embodiments:
said estrogen comprises conjugated estrogens;
said therapeutic agent comprises medroxyprogesterone acetate;
said dissolution profile of said estrogen from said tablet under estrogen dissolution conditions is substantially as shown in any one of FIGS. 9 to 16 and 32 to 46; and
said dissolution profile of said therapeutic agent from said tablet under type I therapeutic agent dissolution conditions is substantially as shown in any one of FIGS. 1 to 8 and 17 to 31.

In some embodiments:
said estrogen comprises conjugated estrogens;
said therapeutic agent comprises 1-[4-(2-azepan-1-yl-ethoxy)-benzyl]-2-(4-hydroxy-phenyl)-3-methyl-1H-indol-5-ol, acetic acid salt;
said dissolution profile of said estrogen from said tablet under estrogen dissolution conditions is substantially as shown in any one of FIGS. 50 to 52; and
said dissolution profile of said therapeutic agent from said tablet under type II therapeutic agent dissolution conditions is substantially as shown in any one of FIGS. 47 to 49.

In some embodiments:
said dissolution profile of said estrogen from said tablet under estrogen dissolution conditions is substantially as shown in any one of FIGS. 9 to 16 and 32 to 46; and
said dissolution profile of said therapeutic agent from said tablet under type I therapeutic agent dissolution conditions is substantially as shown in any one of FIGS. 1 to 8 and 17 to 31.

In some embodiments:
said dissolution profile of said estrogen from said tablet under estrogen dissolution conditions is substantially as shown in any one of FIGS. 50 to 52; and
said dissolution profile of said therapeutic agent from said tablet under type II therapeutic agent dissolution conditions is substantially as shown in any one of FIGS. 47 to 49.

As will be appreciated, some components of the pharmaceutical formulations of the invention can possess multiple functions. For example, a given component can act as both a filler/diluent and a disintegrant. In some such cases, the function of a given component can be considered singular, even though its properties may allow multiple functionality.

### Processes

The present invention is also directed to processes for producing the bi-layer tablets of the invention. Accordingly, in some embodiments, the present invention provides a process for producing a bi-layer tablet of the invention comprising compressing together:
a first mixture comprising at least one estrogen; and
a second mixture comprising a therapeutic agent selected from a selective estrogen receptor modulator and a progestational agent, or mixture thereof.

In some embodiments, the process further comprises:
filling the dies of a compression machine with the second mixture; and
filling the first mixture onto the top of the second mixture.

In some embodiments, the process further comprises:
filling the dies of a compression machine with the first mixture; and
filling the second mixture onto the top of the first mixture.

In some embodiments, the bi-layer tablet produced by the compressing has a hardness from about 8 kp to about 15 kp. In some embodiments, the bi-layer tablet produced by the compressing has a hardness from about 15 kp to about 19 kp.

The first and second mixtures can be prepared by a variety of techniques known to one of ordinary skill in the art. In one aspect, the first or second mixture is prepared by direct blend techniques. In another aspect, the first or second mixture is prepared by wet granulation techniques. In a further aspect, the first or second mixture is prepared by dry granulation processes. Granulation of the mixture can be accomplished by any of the granulation techniques known to one of skill in the art. For example, dry granulation techniques include, but are not limited to, compression of the mixed powder under high pressure, either by roller compaction or “slugging” in a heavy-duty tablet press. Wet granulation techniques include, but are not limited to, high shear granulation, single-pot processing, top-spray granulation, bottom-spray granulation, fluidized spray granulation, extrusion/spheronization, and rotor granulation.

In some embodiments, the process further comprises granulating the first mixture before the compressing.

In some embodiments, the first mixture is prepared by a process comprising the steps of:

(i) mixing the estrogen and a hydrophilic gel-forming polymer component to form an initial mixture;

(ii) granulating the initial mixture to form a first granulated mixture; and

(iii) milling the first granulated mixture.

In some embodiments, step (i) further comprises mixing the estrogen and the hydrophilic gel-forming polymer component with a filler/diluent component and a filler/binder component.

In some embodiments, step (ii) further comprises the steps of:

(x) adding water to the initial mixture during the granulating; and

(y) drying the first granulated mixture before the milling.

In some embodiments, the drying comprises drying the first granulated mixture to a loss on drying (LOD) from about 1.0% to about 3.0%. In some embodiments, the drying comprises drying the first granulated mixture to a loss on drying (LOD) from about 1.5% to about 2.5%. In some embodiments, the drying comprises drying the first granulated mixture to loss on drying (LOD) of about 2%.

In some embodiments, the second mixture is prepared by a process comprising blending the therapeutic agent and a hydrophilic gel-forming polymer component. In some embodiments, the blending of the therapeutic agent and the hydrophilic gel-forming polymer further comprises blending with a filler/diluent component, a filler/binder component, optionally, an antioxidant component and, optionally, a disintegrant. In some embodiments, the process further comprises granulating the second mixture after the blending of the therapeutic agent, the hydrophilic gel-forming polymer filler/diluent component, the filler/binder component, the optional antioxidant component and the optional disintegrant.

In some embodiments, the process further comprises:
blending the first mixture with a lubricant after the milling in step (iii); and
blending the second mixture with a lubricant after the blending of the therapeutic agent, the filler/diluent component, the filler/binder component, the hydrophilic gel-forming polymer component, the optional antioxidant component, if present, and the optional disintegrant component, if present.

The processes described herein can be used to prepare any of the bi-tablets described herein, or combinations or subcombinations thereof.

In some embodiments:
the estrogen comprises conjugated estrogens; and
the second layer comprises one or more therapeutic agents selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

In some embodiments:

(a) the first mixture further comprises:
a filler/diluent component comprising from about 10% to about 90% by weight of the first mixture;
a filler/binder component comprising from about 0.1% to about 30% by weight of the first mixture;
a hydrophilic gel-forming polymer component comprising from about 5% to about 80% by weight of the first mixture; and
optionally, a lubricant component comprising from about 0.01% to about 3% by weight of the first mixture; and

(b) the second mixture further comprises:
a filler/diluent component comprising from about 10% to about 75% by weight of the second mixture;
a filler/binder component comprising up to about 60% by weight of the second mixture;
a hydrophilic gel-forming polymer component comprising from about 1% to about 40% by weight of the second mixture;
optionally, a lubricant component comprising from about 0.01% to about 3% by weight of the second mixture;
optionally, a disintegrant comprising up to about 4% by weight of the second mixture; and
optionally, an antioxidant component comprising from about 0.01% to about 4% by weight of the second mixture.

In some embodiments:
the filler/diluent component of the first mixture comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the filler/binder component of the first mixture comprises one or more of microcrystalline cellulose, polyvinylpyrrolidone, copovidone, polyvinylalcohol, starch, gelatin, gum arabic, gum acacia, and gum tragacanth;
the hydrophilic gel-forming polymer component of the first mixture comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional lubricant component of the first mixture, if present comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the filler/diluent component of the second mixture comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the filler/binder component of the second mixture comprises one or more of microcrystalline cellulose, polyvinylpyrrolidone, copovidone, polyvinylalcohol, starch, gelatin, gum arabic, gum acacia, and gum tragacanth;
the hydrophilic gel-forming polymer of the second mixture comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional lubricant component of the second mixture, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the optional disintegrant of the second mixture, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose; and
the optional antioxidant component of the second mixture, if present, comprises one or more of ascorbic acid, sodium ascorbate, ascorbyl palmitate, butylated hydroxytoluene, and butylated hydroxyanisole.

In some embodiments:
the filler/diluent component of the first mixture comprises one or more of lactose or lactose monohydrate;
the filler/binder component of the first mixture comprises microcrystalline cellulose;
the hydrophilic gel-forming polymer component of the first mixture comprises hydroxypropylmethylcellulose;
the optional lubricant component of the first mixture, if present, comprises magnesium stearate;
the filler/diluent component of the second mixture comprises one or more of lactose or lactose monohydrate;
the filler/binder component of the second mixture comprises microcrystalline cellulose;
the hydrophilic gel-forming polymer component of the second mixture comprises hydroxypropylmethylcellulose;
the optional lubricant component of the second mixture, if present, comprises magnesium stearate;
the optional disintegrant of the second mixture, if present, comprises croscarmellose sodium; and
the optional antioxidant component of the second mixture, if present, comprises one or more of ascorbic acid and vitamin E acetate.

In some embodiments, the filler/diluent component, the filler/binder component, the hydrophilic gel-forming polymer component, or the optional lubricant component of the first mixture are selected from those listed above for the first layer of the bi-layer tablets. In some embodiments, the filler/diluent component, the filler/binder component, the hydrophilic gel-forming polymer component, the optional lubricant component, the optional distintegrant, or the optional antioxidant component of the second mixture are selected from those listed above for the second layer of the bi-layer tablets.

The present invention further provides products produced by the processes of the invention. Any of the embodiments of the processes described herein, or subembodiments or subcombinations thereof, can be used to produce the products of the invention.

In general, the estrogens and therapeutic agents in the bi-layer tablets and mixtures described herein are present in a pharmaceutically effective amount. The phrase “pharmaceutically effective amount” refers to the amount of the active pharmacological agent that elicits the biological or medicinal response in a tissue, system, animal, individual, patient, or human that is being sought by a researcher, veterinarian, medical doctor or other clinician. The desired biological or medicinal response may include preventing the disorder in a patient (e.g., preventing the disorder in a patient that may be predisposed to the disorder, but does not yet experience or display the pathology or symptomatology of the disease). The desired biological or medicinal response may also include inhibiting the disorder in a patient that is experiencing or displaying the pathology or symptomatology of the disorder (i.e., arresting or slowing further development of the pathology and/or symptomatology). The desired biological or medicinal response may also include ameliorating the disorder in a patient that is experiencing or displaying the pathology or symptomatology of the disease (i.e., reversing the pathology or symptomatology).

The pharmaceutically effective amount provided in the propylaxis or treatment of a specific disorder may vary according to the specific condition(s) being treated, the size, age and response pattern of the patient, the severity of the disorder, the judgment of the attending physician or the like. In general, effective amounts for daily oral administration may be about 0.01 mg/kg to 1,000 mg/kg, for example, about 0.5 mg/kg to 500 mg/kg and effective amounts for parenteral administration may be about 0.1 to 100 mg/kg, for example, about 0.5 mg/kg to 50 mg/kg.

In general, the pharmaceutical formulations, and compositions thereof, can be administered by any appropriate route, for example, orally. The excipients of the bi-layer tablets and mixtures can also be combined with mixtures of other active compounds or inert fillers and/or diluents. Additional numerous various excipients, dosage forms, dispersing agents and the like that are suitable for use in connection with the tablets of the invention are known in the art and described in, for example, Remington's Pharmaceutical Sciences, 17th ed., Mack Publishing Company, Easton, Pa., 1985, which is incorporated herein by reference in its entirety.

Film coatings useful with the present formulations are known in the art and generally consist of a polymer (usually a cellulosic type of polymer), a colorant and a plasticizer. Additional ingredients such as wetting agents, sugars, flavors, oils and lubricants can be included in film coating formulations to impart certain characteristics to the film coat. The compositions and formulations herein may also be combined and processed as a solid, then placed in a capsule form such as a gelatin capsule.

Certain features of the invention are described herein in embodiments. It is emphasized that certain features of the invention, which are, for clarity, described herein in the context of separate embodiments, can also be provided in combination in a single embodiment, unless otherwise specified. Conversely, various features of the invention which are, for brevity, described in the context of a single embodiment, can also be provided separately or in any suitable subcombination, unless otherwise specified. For example, some of the embodiments herein describe individual weight percentages for each excipient, estrogen, or therapeutic agent in a given layer or mixture, while other embodiments herein describe the chemical composition of the excipients, estrogens, or therapeutic agents; these embodiments can also be provided in any suitable combination or subcombination, as well as being provided separately in a single embodiment, unless otherwise specified.

In order that the invention disclosed herein may be more efficiently understood, examples are provided below. It should be understood that these examples are for illustrative purposes only and are not to be construed as limiting the invention in any manner.

### EXAMPLES

### Example 1

### Granulation Comprising Conjugated Estrogens and 27.5% HPMC K100M Premium CR

Using the ingredients in the amounts shown in Table I, a conjugated estrogens desiccation with lactose (CEDL) at 42.9 mg/g mixture was granulated with the other ingredients using water in a high shear granulator using the procedure below for a batch size of 1.5 kg.

1. CEDL was mixed with Lactose Spray Dried, Avicel®, and HPMC in a Collette shear mixer for approximately 5 minutes with plows at approximately 430 rpm.
2. The blend of step 1 was granulated by initiating the addition of water with plows and choppers set at approximately 430 and 1800 rpm, respectively. Add all the water within approximately 4 minutes.
3. Granulation was continued for total of approximately 7 minutes.
4. The wet granulation was dried in a fluid bed dryer at an inlet temperature set point of 60° C. to achieve a target granulation LOD of 2%. A variation of ±0.5% moisture content is acceptable.
5. The dried granulation was passed through a Model “M” Fitzmill equipped with a # 2A plate, set at a high speed (4500-4600 rpm), and impact set forward.
6. The granulation of step 5 was mixed in a V-Blender for approximately 10 minutes at approximately 22 rpm.
7. About 100 g of the blend of step 6 was removed for use in Step 8.
8. Magnesium stearate (MS) was added through a # 20 screen, in approximately equal portions, to each side of the V-blender. After the magnesium stearate addition, the step 7 blend was added in approximately equal portions, to each side of V-Blender. This mixture was blended for approximately 3 minutes. The quantity of magnesium stearate added was adjusted on a per tablet basis based on the quantity of granulation to be blended.
9. The step 8 lubricated granulation was discharged into a double-bagged polyethylene bag with a desiccant bag in between the bags.

TABLE I

| | Input/Tablet | |
|---|---|---|
| Description | (mg) | % W/W |
| CE Desiccation with Lactose at 42.9 mg/g | 10.4895 | 8.74 |
| Lactose Monohydrate Spray Dried | 58.2105 | 48.51 |
| Microcrystalline Cellulose (Avicel PH 101, NF) | 18 | 15.00 |
| Hydroxypropylmethylcellulose (HPMC K100M | 33 | 27.50 |
| Premium CR) | | |
| Magnesium Stearate, NF | 0.3 | 0.25 |
| Purified Water, USP (A) | 30 | |

Note:
(A) Indicates removed during processing.

### Example 2

### Granulation Comprising Conjugated Estrogens and 10% HPMC K100M Premium CR

Using the ingredients in the amounts shown in Table II, a conjugated estrogens desiccation with lactose (CEDL) at 42.9 mg/g mixture was granulated with the other ingredients by following the procedure of Example 1.

TABLE II

| | Input/Tablet | |
|---|---|---|
| Description | (mg) | % W/W |
| CE Desiccation with Lactose at 42.9 mg/g | 10.4895 | 8.74 |
| Lactose Monohydrate Spray Dried | 79.21 | 66.01 |
| Microcrystalline Cellulose (Avicel PH 101, NF) | 18 | 15.00 |
| Hydroxypropylmethylcellulose (HPMC K100M | 12 | 10.00 |
| Premium CR) | | |
| Magnesium Stearate, NF | 0.3 | 0.25 |
| Purified Water, USP (A) | 30 | |

Note:
(A) Indicates removed during processing.

### Example 3

### Granulation Comprising Conjugated Estrogens and 20% HPMC K100M Premium CR

Using the ingredients in the amounts shown in Table III, a conjugated estrogens desiccation with lactose (CEDL) at 42.9 mg/g mixture was granulated with the other ingredients by following the procedure of Example 1.

TABLE III

| | Input/Tablet | |
|---|---|---|
| Description | (mg) | % W/W |
| CE Desiccation with Lactose at 42.9 mg/g | 10.4895 | 8.74 |
| Lactose Monohydrate Spray Dried | 67.21 | 56.01 |
| Microcrystalline Cellulose (Avicel PH 101, NF) | 18 | 15.00 |
| Hydroxypropylmethylcellulose (HPMC K100M | 24 | 20.00 |
| Premium CR) | | |
| Magnesium Stearate, NF | 0.3 | 0.25 |
| Purified Water, USP (A) | 30 | |

Note:
(A) Indicates removed during processing.

### Example 4

### Granulation Comprising Conjugated Estrogens and 35% HPMC K100M Premium CR

Using the ingredients in the amounts shown in Table IV, a conjugated estrogens desiccation with lactose (CEDL) at 42.9 mg/g mixture was granulated with the other ingredients by following the procedure of Example 1.

TABLE IV

| | Input/Tablet | |
|---|---|---|
| Description | (mg) | % W/W |
| CE Desiccation with Lactose at 42.9 mg/g | 10.4895 | 8.74 |
| Lactose Monohydrate Spray Dried | 49.212 | 41.01 |
| Microcrystalline Cellulose (Avicel PH 101, NF) | 18 | 15.00 |
| Hydroxypropylmethylcellulose (HPMC K100M | 42 | 35.00 |
| Premium CR) | | |
| Magnesium Stearate, NF | 0.3 | 0.25 |
| Purified Water, USP (A) | 30 | |

Note:
(A) Indicates removed during processing.

### Example 5

### Granulation Comprising Conjugated Estrogens and 45% HPMC K100M Premium CR

For formulations of CE with 45 and 55% HPMC K100M CR, since the wet granulation process produced very hard granules due to the high levels of HPMC K100M in these two formulations, it was difficult to dry the wet granulations to the desired moisture level. Therefore, a dry granulation method was employed. Accordingly, using the ingredients in the amounts shown in Table V, a conjugated estrogens desiccation with lactose (CEDL) at 42.9 mg/g mixture was granulated with the other ingredients by following the procedure below for a batch size of 1 kg.

1. The intra-granular excipients, except for magnesium stearate, were screened through a #30 mesh screen and then blended in a 4 quart V-blender for approximately 15 minutes at about 22 rpm.
2. The intra-granular magnesium stearate was added to the blender and blended for approximately 3 minutes at about 22 rpm.
3. The step 2 blend was granulated using Fitzpatrick Chilsonator IR 220 at following parameters:
Roll Pressure: approximately 309 psi
Roll Force: approximately 2556 lb/in
Roll Speed: approximately 7 rpm
VFS: approximately 180 rpm
HFS: approximately 25 rpm
   4. The ribbon was milled using a Quadro Comil 197S at about 20% motor speed using screen with about 1.575 mm opening.
   5. The milled material were then weighed.
   6. The milled materials were blended in the 4 Qt V-blender for approximately 5 minutes at about 22 rpm.
   7. The quantity of extra-granular magnesium stearate needed was calculated based on the yield.
   8. The magnesium stearate was weighed and added to the blender and then blended for approximately 3 minutes at about 22 rpm.

TABLE V

| | | |
|---|---|---|
| | Input/Tablet | |
| Description | (mg) | % W/W |

Intra-Granular

| | | |
|---|---|---|
| CE Desiccation with Lactose at 42.9 mg/g | 10.4895 | 8.74 |
| Lactose Monohydrate Spray Dried | 37.2105 | 31.01 |
| Microcrystalline Cellulose (Avicel PH 101, NF) | 18 | 15.00 |
| Hydroxypropylmethylcellulose (HPMC K100M | 54 | 45.00 |
| Premium CR) | | |
| Magnesium Stearate, NF | 0.12 | 0.10 |

Extra-Granular

| | | |
|---|---|---|
| Magnesium Stearate, NF | 0.18 | 0.15 |

### Example 6

### Granulation Comprising Conjugated Estrogens and 55% HPMC K100M Premium CR

Using the ingredients in the amounts shown in Table VI, a conjugated estrogens desiccation with lactose (CEDL) at 42.9 mg/g mixture was granulated with the other ingredients by following the procedure of Example 5.

TABLE VI

| | | |
|---|---|---|
| | Input/Tablet | |
| Description | (mg) | % W/W |

Intra-Granular

| | | |
|---|---|---|
| CE Desiccation with Lactose at 42.9 mg/g | 10.4895 | 8.74 |
| Lactose Monohydrate Spray Dried | 25.2105 | 21.01 |
| Microcrystalline Cellulose (Avicel PH 101, NF) | 18 | 15.00 |
| Hydroxypropylmethylcellulose (HPMC K100M | 66 | 55.00 |
| Premium CR) | | |
| Magnesium Stearate, NF | 0.12 | 0.10 |

Extra-Granular

| | | |
|---|---|---|
| Magnesium Stearate, NF | 0.18 | 0.15 |

### Example 7

### Blend Comprising Medroxyprogesterone Acetate and 20% HPMC K4M Premium CR

Using the ingredients in the amounts shown in Table VII, medroxyprogesterone acetate (MPA) was blended with the rest of ingredients utilizing the following procedure.

1. MPA was screen together with Avicel® PH 200 through a #20 mesh screen.
2. The step 1 mixture was blended in a V-blender for approximately 110 revolutions.
3. Lactose monohydrate, HPMC, and the other excipients except magnesium stearate were screened through the same screen and add to the blender.
4. The mixture of step 3 was blended for approximately 330 revolutions.
5. The magnesium stearate was screened together with about 100 g of blend from step 4 through the same screen and add to the blender. This mixture was then blended for approximately 66 revolutions.

TABLE VII

| | Input/Tablet | |
|---|---|---|
| Description | (mg) | % W/W |
| Medroxyprogesterone Acetate, USP, | 1.5 | 0.63 |
| Micronized @100% | | |
| Lactose Monohydrate Spray Dried, NF | 93.9 | 39.13 |
| Microcrystalline Cellulose, NF (Avicel PH 200) | 96 | 40.00 |
| Hydroxypropylmethylcellulose, HPMC K4M | 48 | 20.00 |
| Premium CR | | |
| Magnesium Stearate, NF | 0.6 | 0.25 |

### Example 8

### Blend Comprising Medroxyprogesterone Acetate, 10% HPMC K4M Premium CR, and 10% HPMC K100M Premium CR

Using the ingredients in the amounts shown in Table VIII, medroxyprogesterone acetate (MPA) was blended with the rest of ingredients utilizing the procedure of Example 7.

TABLE VIII

| | Input/Tablet | |
|---|---|---|
| Description | (mg) | % W/W |
| Medroxyprogesterone Acetate, USP, | 1.5 | 0.63 |
| Micronized @100% | | |
| Lactose Monohydrate Spray Dried, NF | 93.9 | 39.13 |
| Microcrystalline Cellulose, NF (Avicel PH 200) | 96 | 40.00 |
| Hydroxypropylmethylcellulose, HPMC K4M | 24 | 10.00 |
| Premium CR | | |
| Hydroxypropylmethylcellulose, HPMC K100M | 24 | 10.00 |
| Premium CR | | |
| Magnesium Stearate, NF | 0.6 | 0.25 |

### Example 9

### Blend Comprising Medroxyprogesterone Acetate and 20% HPMC K100M Premium CR

Using the ingredients in the amounts shown in Table IX medroxyprogesterone acetate (MPA) was blended with the rest of ingredients utilizing the procedure of Example 7.

TABLE IX

| | Input/Tablet | |
|---|---|---|
| Description | (mg) | % W/W |
| Medroxyprogesterone Acetate, USP, | 1.5 | 0.63 |
| Micronized @100% | | |
| Lactose Monohydrate Spray Dried, NF | 93.9 | 39.13 |
| Microcrystalline Cellulose, NF (Avicel PH 200) | 96 | 40.00 |
| Hydroxypropylmethylcellulose, HPMC K100M | 48 | 20.00 |
| Premium CR | | |
| Magnesium Stearate, NF | 0.6 | 0.25 |

### Example 10

### Blend Comprising Medroxyprogesterone Acetate, 20% HPMC K100M Premium CR, and Croscarmellose Sodium

Using the ingredients in the amounts shown in Table X, medroxyprogesterone acetate (MPA) was blended with the rest of ingredients utilizing the procedure of Example 7.

TABLE X

| | Input/Tablet | |
|---|---|---|
| Description | (mg) | % W/W |
| Medroxyprogesterone Acetate, USP, | 1.5 | 0.63 |
| Micronized @100% | | |
| Lactose monohydrate spray dried | 91.5 | 38.13 |
| Microcrystalline Cellulose, NF (Avicel PH 200) | 96 | 40.00 |
| Hydroxypropylmethylcellulose, HPMC K100M | 48 | 20.00 |
| Premium CR | | |
| Croscarmellose Sodium (Ac-Di-Sol, NF) | 2.4 | 1.00 |
| Magnesium Stearate, NF | 0.6 | 0.25 |

### Example 11

### Blend Comprising Medroxyprogesterone Acetate, 10% HPMC 100M Premium CR, and 10% HPMC LV100

Using the ingredients in the amounts shown in Table XI, medroxyprogesterone acetate (MPA) was blended with the rest of ingredients utilizing the procedure of Example 7.

TABLE XI

| | Input/Tablet | |
|---|---|---|
| Description | (mg) | % W/W |
| Medroxyprogesterone Acetate, USP, | 1.5 | 0.63 |
| Micronized @100% | | |
| Lactose monohydrate spray dried | 93.9 | 39.13 |
| Microcrystalline Cellulose, NF (Avicel PH 200) | 96 | 40.00 |
| Hydroxypropylmethylcellulose, HPMC K100M | 24 | 10.00 |
| Premium CR | | |
| Hydroxypropylmethylcellulose, HPMC LV100 | 24 | 10.00 |
| Magnesium Stearate, NF | 0.6 | 0.25 |

### Example 12

### Blend Comprising Medroxyprogesterone Acetate and 10% HPMC 100M Premium CR

Using the ingredients in the amounts shown in Table XII, medroxyprogesterone acetate (MPA) was blended with the rest of ingredients utilizing the procedure of Example 7.

TABLE XII

| | Input/Tablet | |
|---|---|---|
| Description | (mg) | % W/W |
| Medroxyprogesterone Acetate, USP, | 1.5 | 0.63 |
| Micronized @100% | | |
| Lactose monohydrate spray dried | 117.9 | 49.13 |
| Microcrystalline Cellulose, NF (Avicel PH 200) | 96 | 40.00 |
| Hydroxypropylmethylcellulose, HPMC K100M | 24 | 10.00 |
| Premium CR | | |
| Magnesium Stearate, NF | 0.6 | 0.25 |

### Example 13

### Blend Comprising Medroxyprogesterone Acetate and 5% HPMC 4M Premium CR

Using the ingredients in the amounts shown in Table XIII, medroxyprogesterone acetate (MPA) was blended with the rest of ingredients utilizing the procedure of Example 7.

TABLE XIII

| | Input/Tablet | |
|---|---|---|
| Description | (mg) | % W/W |
| Medroxyprogesterone Acetate, USP, | 1.5 | 0.63 |
| Micronized @100% | | |
| Lactose monohydrate spray dried | 129.9 | 54.13 |
| Microcrystalline Cellulose, NF (Avicel PH 200) | 96 | 40.00 |
| Hydroxypropylmethylcellulose, HPMC K4M | 12 | 5.00 |
| Premium CR | | |
| Magnesium Stearate, NF | 0.6 | 0.25 |

### Example 14

### Blend Comprising Medroxyprogesterone Acetate and 5% HPMC 100M Premium CR

Using the ingredients in the amounts shown in Table XIV, medroxyprogesterone acetate (MPA) was blended with the rest of ingredients utilizing the procedure of Example 7.

TABLE IV

| | Input/Tablet | |
|---|---|---|
| Description | (mg) | % W/W |
| Medroxyprogesterone Acetate, USP, | 1.5 | 0.63 |
| Micronized @100% | | |
| Lactose monohydrate spray dried | 129.9 | 54.13 |
| Microcrystalline Cellulose, NF (Avicel PH 200) | 96 | 40.00 |
| Hydroxypropylmethylcellulose, HPMC K100M | 12 | 5.00 |
| Premium CR | | |
| Magnesium Stearate, NF | 0.6 | 0.25 |

### Example 15A

### Compression of Bi-Layer Tablets Using CE Granulation and MPA Blends

Using the CE granulation of Example 1 and the MPA blend of Example 7, a CE/MPA bi-layer tablet was compressed using a Kilian RUD compression machine with 11 mm round convex tooling. The targeted total bi-layer tablet weight was 360 mg with 240 mg and 120 mg for the MPA and CE sub-layer portion, respectively. The compression force was adjusted in order to get bi-layer tablets within the targeted hardness range of 8-15 kp. Under this compression force the bi-layer tablet had a friability of zero percent.

The compression machine was setup with MPA blend filling into the dies first then CE granulation on the top of MPA layer. During the compression, vacuum was applied between fill cams of MPA blend and CE granulation in order to prevent MPA blend from being filled into the CE layer and CE granulation being filled into the MPA layer.

For stability evaluations, the tablets were coated with Opadry® White to approximately 5% weight gain using the Vector Coater LDCS 3 with a 1.3 Liter pan insert.

### Example 15B

### Compression of Bi-Layer Tablets Using CE Granulation and MPA Blends

Using the CE granulation of Example 1 and the MPA blend of Example 8, a CE/MPA bi-layer tablet was prepared by following the procedure of Example 15A.

### Example 15C

### Compression of Bi-Layer Tablets Using CE Granulation and MPA Blends

Using the CE granulation of Example 1 and the MPA blend of Example 9, a CE/MPA bi-layer tablet was prepared by following the procedure of Example 15A.

### Example 15D

### Compression of Bi-Layer Tablets Using CE Granulation and MPA Blends

Using the CE granulation of Example 1 and the MPA blend of Example 10, a CE/MPA bi-layer tablet was prepared by compression using a Kilian RUD compression machine with 11 mm round convex tooling. The machine was filled with CE granulation first then MPA blend. The targeted total bi-layer tablet weight was 360 mg with 240 mg and 120 mg for the MPA and CE sub-layer portion, respectively. The compression force was adjusted in order to get bi-layer tablets within the targeted hardness range of 8-15 kp. Under this compression force the bi-layer tablet had a friability of zero percent.

### Example 15E

### Compression of Bi-Layer Tablets Using CE Granulation and MPA Blends

Using the CE granulation of Example 1 and the MPA blend of Example 11, a CE/MPA bi-layer tablet was prepared by following the procedure of Example 15D.

### Example 15F

### Compression of Bi-Layer Tablets Using CE Granulation and MPA Blends

Using the CE granulation of Example 1 and the MPA blend of Example 12, a CE/MPA bi-layer tablet was prepared by following the procedure of Example 15D.

### Example 15G

### Compression of Bi-Layer Tablets Using CE Granulation and MPA Blends

Using the CE granulation of Example 1 and the MPA blend of Example 13, a CE/MPA bi-layer tablet was prepared by following the procedure of Example 15D.

### Example 15H

### Compression of Bi-Layer Tablets Using CE Granulation and MPA Blends

Using the CE granulation of Example 1 and the MPA blend of Example 14, a CE/MPA bi-layer tablet was prepared by following the procedure of Example 15D.

### Example 16

### Compression of Bi-Layer Tablets Using CE Granulation and MPA Blends

Using the CE granulation of Example 1 and the MPA blend of Example 12, a CE/MPA bi-layer tablet was prepared by following the procedure of Example 15D. The tablets were coated with Opadry® White to approximately 5% weight gain using the Vector Coater LDCS 3 with a 1.3 Liter pan insert.

### Example 17

### Content Uniformity and Weight Variation of MPA and CE of Bi-Layer Tablets

Content uniformity of MPA and CE was determined on a sample size of 10 tablets. Weight variation of 100 tablets was evaluated using the Mocon Automatic Balance Analysis tester. The results are shown in Table XV.

TABLE XV

| | |
|---|---|
| Content Uniformity | |
| (%) | Weight |

| EXAMPLE # | MPA | CE | Variation (%) |
|---|---|---|---|
| EXAMPLE 15A | 0.77 | 4.75 | 1.22 |
| EXAMPLE 15B | 1.34 | 6.50 | 1.39 |
| EXAMPLE 15C | 0.94 | 7.20 | 1.85 |
| EXAMPLE 15D | 1.65 | 2.66 | 2.18 |
| EXAMPLE 15E | 2.94 | 2.48 | 1.04 |
| EXAMPLE 15F | 1.07 | 2.47 | 0.72 |
| EXAMPLE 15G | 1.99 | 4.32 | 1.43 |
| EXAMPLE 15H | 1.15 | 1.98 | 0.98 |
| EXAMPLE 16 | 0.81 | 2.41 | 0.90 |

### Example 18

### Dissolution of MPA from the Bi-Layer Tablets

The dissolution of MPA for Examples 15A-15H was determined using USP Apparatus 2, at 50 rpm in 900 mL with 0.54% Sodium Lauryl Sulfate (SLS) in water for a period of 12 hours. A filtered sample of the dissolution medium was taken at specified time intervals. The release of the active was determined by reversed phase high performance liquid chromatography (HPLC). The results are shown in Table XVI and FIGS. 1 to 8.

TABLE XVI

| Example | Percent Released at Different Time Intervals (% ± sd, n = 6) | | | | | | | |
|---|---|---|---|---|---|---|---|---|
| # | 0 min | 15 min | 30 min | 45 min | 60 min | 120 min | 360 min | 720 min |
| 15A | 0 | 5 ± 1.7 | 7 ± 2.3 | 9 ± 2.7 | 12 ± 3.0 | 21 ± 4.1 | 54 ± 4.8 | 91 ± 4.2 |
| 15B | 0 | 2 ± 0.5 | 4 ± 0.8 | 6 ± 1.3 | 8 ± 1.7 | 16 ± 3.5 | 49 ± 8.6 | 87 ± 7.0 |
| 15C | 0 | 2 ± 0.2 | 3 ± 0.3 | 5 ± 0.4 | 6 ± 0.5 | 12 ± 1.4 | 40 ± 2.7 | 81 ± 5.7 |
| 15D | 0 | 2 ± 0.6 | 4 ± 0.9 | 5 ± 1.1 | 7 ± 1.4 | 14 ± 2.4 | 43 ± 4.5 | 78 ± 2.2 |
| 15E | 0 | 2 ± 0.3 | 3 ± 0.5 | 5 ± 0.8 | 7 ± 1.0 | 15 ± 2.2 | 49 ± 4.9 | 92 ± 3.8 |
| 15F | 0 | 13 ± 7.1 | 19 ± 8.5 | 24 ± 9.4 | 28 ± 9.8 | 40 ± 10.6 | 79 ± 8.9 | 99 ± 1.2 |
| 15G | 0 | 44 ± 5.0 | 56 ± 7.0 | 64 ± 7.9 | 69 ± 9.1 | 83 ± 10.9 | 95 ± 4.5 | 98 ± 3.2 |
| 15H | 0 | 35 ± 13.8 | 46 ± 15.8 | 54 ± 16.7 | 60 ± 16.7 | 72 ± 15.2 | 93 ± 6.7 | 98 ± 0.91 |

### Example 19

### Dissolution of CE from the Bi-Layer Tablets

The dissolution of CE for Examples 15A-15H was determined using USP Apparatus 2, at 50 rpm in 900 mL of 0.02M Sodium Acetate Buffer, pH 4.5 for a period of 8 hours. Filtered samples of the dissolution medium were taken at specified time intervals. The release of the active was determined on a reversed phase high performance liquid chromatography (HPLC). The results are shown in Table XVII and FIGS. 9 to 16.

TABLE XVII

| | Percent Released at Different Time Intervals | | | |
|---|---|---|---|---|
| | (% ± sd, n = 6) | | | |
| Formulation | 0 (hr) | 2 (hr) | 5 (hr) | 8 (hr) |
| EXAMPLE 15A | 0 | 43 ± 4.5 | 73 ± 4.2 | 90 ± 5.1 |
| EXAMPLE 15B | 0 | 42 ± 4.1 | 70 ± 5.7 | 86 ± 5.7 |
| EXAMPLE 15C | 0 | 41 ± 4.8 | 68 ± 5.7 | 84 ± 3.9 |
| EXAMPLE 15D | 0 | 37 ± 4.7 | 66 ± 5.1 | 83 ± 2.9 |
| EXAMPLE 15E | 0 | 40 ± 5.3 | 67 ± 4.8 | 83 ± 3.6 |
| EXAMPLE 15F | 0 | 47 ± 3.0 | 76 ± 2.8 | 91 ± 3.6 |
| EXAMPLE 15G | 0 | 65 ± 4.2 | 97 ± 3.9 | 103 ± 4.5 |
| EXAMPLE 15H | 0 | 52 ± 6.3 | 87 ± 3.9 | 97 ± 2.7 |

### Example 20A

### Bi-Layer Tablet Prepared from CE Granulation and MPA Blend

Using the CE granulation of Example 4 and MPA blend of Example 9, bi-layer tablets were prepared according to the procedure of Example 15D. Table XVIII shows the composition of the bi-layer tablet.

### Example 20B

### Bi-Layer Tablet Prepared from CE Granulation and MPA Blend

Using the CE granulation of Example 2 and MPA blend of Example 9, bi-layer tablets were prepared according to the procedure of Example 15D. Table XVIII shows the composition of the bi-layer tablet.

### Example 20C

### Bi-Layer Tablet Prepared from CE Granulation and MPA Blend

Using the CE granulation of Example 4 and MPA blend of Example 14, bi-layer tablets were prepared according to the procedure of Example 15D. Table XVIII shows the composition of the bi-layer tablet.

### Example 20D

### Bi-Layer Tablet Prepared from CE Granulation and MPA Blend

Using the CE granulation of Example 2 and MPA blend of Example 14, bi-layer tablets were prepared according to the procedure of Example 15D. Table XVIII shows the composition of the bi-layer tablet.

### Example 20E

### Bi-Layer Tablet Prepared from CE Granulation and MPA Blend

Using the CE granulation of Example 4 and MPA blend of Example 12, bi-layer tablets were prepared according to the procedure of Example 15D. Table XVIII shows the composition of the bi-layer tablet.

### Example 20F

### Bi-Layer Tablet Prepared from CE Granulation and MPA Blend

Using the CE granulation of Example 6 and MPA blend of Example 14, bi-layer tablets were prepared according to the procedure of Example 15D. Table XIX shows the composition of the bi-layer tablet.

### Example 20G

### Bi-Layer Tablet Prepared from CE Granulation and MPA Blend

Using the CE granulation of Example 6 and MPA blend of Example 12, bi-layer tablets were prepared according to the procedure of Example 15D. Table XIX shows the composition of the bi-layer tablet.

### Example 20H

### Bi-Layer Tablet Prepared from CE Granulation and MPA Blend

Using the CE granulation of Example 6 and MPA blend of Example 9, bi-layer tablets were prepared according to the procedure of Example 15D. Table XIX shows the composition of the bi-layer tablet.

### Example 20I

### Bi-Layer Tablet Prepared from CE Granulation and MPA Blend

Using the CE granulation of Example 2 and MPA blend of Example 12, bi-layer tablets were prepared according to the procedure of Example 15D. Table XIX shows the composition of the bi-layer tablet.

### Example 20J

### Bi-Layer Tablet Prepared from CE Granulation and MPA Blend

Using the CE granulation of Example 3 and MPA blend of Example 9, bi-layer tablets were prepared according to the procedure of Example 15D. Table XIX shows the composition of the bi-layer tablet.

### Example 20K

### Bi-Layer Tablet Prepared from CE Granulation and MPA Blend

Using the CE granulation of Example 3 and MPA blend of Example 14, bi-layer tablets were prepared according to the procedure of Example 15D. Table XX shows the composition of the bi-layer tablet.

### Example 20L

### Bi-Layer Tablet Prepared from CE Granulation and MPA Blend

Using the CE granulation of Example 3 and MPA blend of Example 12, bi-layer tablets were prepared according to the procedure of Example 15D. Table XX shows the composition of the bi-layer tablet.

### Example 20M

### Bi-Layer Tablet Prepared from CE Granulation and MPA Blend

Using the CE granulation of Example 5 and MPA blend of Example 14, bi-layer tablets were prepared according to the procedure of Example 15D. Table XX shows the composition of the bi-layer tablet.

### Example 20N

### Bi-Layer Tablet Prepared from CE Granulation and MPA Blend

Using the CE granulation of Example 5 and MPA blend of Example 12, bi-layer tablets were prepared according to the procedure of Example 15D. Table XX shows the composition of the bi-layer tablet.

### Example 20O

### Bi-Layer Tablet Prepared from CE Granulation and MPA Blend

Using the CE granulation of Example 5 and MPA blend of Example 9, bi-layer tablets were prepared according to the procedure of Example 15D. Table XX shows the composition of the bi-layer tablet.

TABLE XVIII

| | Input/Tablet (mg) (% W/W) | | | | |
|---|---|---|---|---|---|
| | Example | Example | Example | Example | Example |
| Ingredients | 20A | 20B | 20C | 20D | 20E |

CE Layer

| | | | | | | | | | | |
|---|---|---|---|---|---|---|---|---|---|---|
| CE Desiccation | 10.49 | (8.74%) | 10.49 | (8.74%) | 10.49 | (8.74%) | 10.49 | (8.74%) | 10.49 | (8.74%) |
| w/Lactose | | | | | | | | | | |
| Lactose | 49.21 | (41.01%) | 79.21 | (66.01%) | 49.21 | (41.01%) | 79.21 | (66.01%) | 49.21 | (41.01%) |
| Monohydrate, | | | | | | | | | | |
| Spray Dried, NF | | | | | | | | | | |
| Microcrystalline | 18 | (15%) | 18 | (15%) | 18 | (15%) | 18 | (15%) | 18 | (15%) |
| Cellulose, NF, | | | | | | | | | | |
| (Avicel PH 101) | | | | | | | | | | |
| HPMC K100M | 42 | (35%) | 12 | (10%) | 42 | (35%) | 12 | (10%) | 42 | (35%) |
| Premium CR | | | | | | | | | | |
| Magnesium | 0.3 | (0.25%) | 0.3 | (0.25%) | 0.3 | (0.25%) | 0.3 | (0.25%) | 0.3 | (0.25%) |
| Stearate, NF | | | | | | | | | | |

MPA Layer

| | | | | | | | | | | |
|---|---|---|---|---|---|---|---|---|---|---|
| MPA, USP, | 1.5 | (0.63%) | 1.5 | (0.63%) | 1.5 | (0.63%) | 1.5 | (0.63%) | 1.5 | (0.63%) |
| Micronized, 100% | | | | | | | | | | |
| Lactose | 93.9 | (39.13%) | 93.9 | (39.13) | 129.9 | (54.13) | 129.9 | (54.13) | 117.9 | (49.13%) |
| Monohydrate, | | | | | | | | | | |
| Spray Dried, NF | | | | | | | | | | |
| Microcrystalline | 96 | (40%) | 96 | (40%) | 96 | (40%) | 96 | (40%) | 96 | (40%) |
| Cellulose, NF, | | | | | | | | | | |
| (Avicel PH 200) | | | | | | | | | | |
| HPMC K100M | 48 | (20%) | 48 | (20%) | 12 | (5%) | 12 | (5%) | 24 | (10%) |
| Premium CR | | | | | | | | | | |
| Magnesium | 0.6 | (0.25%) | 0.6 | (0.25%) | 0.6 | (0.25%) | 0.6 | (0.25%) | 0.6 | (0.25%) |
| Stearate, NF | | | | | | | | | | |

TABLE XIX

| | Input/Tablet (mg) (% W/W) | | | | |
|---|---|---|---|---|---|
| | Example | Example | Example | Example | Example |
| Ingredients | 20F | 20G | 20H | 20I | 20J |

CE Layer

| | | | | | | | | | | |
|---|---|---|---|---|---|---|---|---|---|---|
| CE Desiccation | 10.49 | (8.74%) | 10.49 | (8.74%) | 10.49 | (8.74%) | 10.49 | (8.74%) | 10.4895 | (8.74%) |
| w/Lactose | | | | | | | | | | |
| Lactose | 25.21 | (21.01%) | 25.21 | (21.01%) | 25.21 | (21.01%) | 79.21 | (66.01%) | 67.2105 | (56.01%) |
| Monohydrate, | | | | | | | | | | |
| Spray Dried, NF | | | | | | | | | | |
| Microcrystalline | 18 | (15%) | 18 | (15%) | 18 | (15%) | 18 | (15%) | 18 | (15%) |
| Cellulose, NF, | | | | | | | | | | |
| (Avicel PH 101) | | | | | | | | | | |
| HPMC K100M | 66 | (55%) | 66 | (55%) | 66 | (55%) | 12 | (10%) | 24 | (20%) |
| Premium CR | | | | | | | | | | |
| Magnesium | 0.3 | (0.25%) | 0.3 | (0.25%) | 0.3 | (0.25%) | 0.3 | (0.25%) | 0.3 | (0.25) |
| Stearate, NF | | | | | | | | | | |

MPA Layer

| | | | | | | | | | | |
|---|---|---|---|---|---|---|---|---|---|---|
| MPA, USP, | 1.5 | (0.63%) | 1.5 | (0.63%) | 1.5 | (0.63%) | 1.5 | (0.63%) | 1.5 | (0.625%) |
| Micronized, 100% | | | | | | | | | | |
| Lactose | 129.9 | (54.13) | 117.9 | (49.13%) | 93.9 | (39.13) | 117.9 | (49.13%) | 93.9 | (39.125%) |
| Monohydrate, | | | | | | | | | | |
| Spray Dried, NF | | | | | | | | | | |
| Microcrystalline | 96 | (40%) | 96 | (40%) | 96 | (40%) | 96 | (40%) | 96 | (40%) |
| Cellulose, NF, | | | | | | | | | | |
| (Avicel PH 200) | | | | | | | | | | |
| HPMC K100M | 12 | (5%) | 24 | (10%) | 48 | (20%) | 24 | (10%) | 48 | (20%) |
| Premium CR | | | | | | | | | | |
| Magnesium | 0.6 | (0.25%) | 0.6 | (0.25%) | 0.6 | (0.25%) | 0.6 | (0.25%) | 0.6 | (0.25%) |
| Stearate, NF | | | | | | | | | | |

TABLE XX

| | Input/Tablet (mg) (% W/W) | | | | |
|---|---|---|---|---|---|
| | Example | Example | Example | Example | Example |
| Ingredients | 20K | 20L | 20M | 20N | 20O |

CE Layer

| | | | | | | | | | | |
|---|---|---|---|---|---|---|---|---|---|---|
| CE Desiccation | 10.4895 | (8.74%) | 10.4895 | (8.74%) | 10.4895 | (8.74%) | 10.4895 | (8.74%) | 10.4895 | (8.74%) |
| w/Lactose | | | | | | | | | | |
| Lactose | 67.2105 | (56.01%) | 67.2105 | (56.01%) | 37.2105 | (31.01%) | 37.2105 | (31.01%) | 37.2105 | (31.01%) |
| Monohydrate, | | | | | | | | | | |
| Spray Dried, NF | | | | | | | | | | |
| Microcrystalline | 18 | (15%) | 18 | (15%) | 18 | (15%) | 18 | (15%) | 18 | (15%) |
| Cellulose, NF, | | | | | | | | | | |
| (Avicel PH 101) | | | | | | | | | | |
| HPMC K100M | 24 | (20%) | 24 | (20%) | 54 | (45%) | 54 | (45%) | 54 | (45%) |
| Premium CR | | | | | | | | | | |
| Magnesium | 0.3 | (0.25) | 0.3 | (0.25) | 0.3 | (0.25%) | 0.3 | (0.25%) | 0.3 | (0.25%) |
| Stearate, NF | | | | | | | | | | |

MPA Layer

| | | | | | | | | | | |
|---|---|---|---|---|---|---|---|---|---|---|
| MPA, USP, | 1.5 | (0.625%) | 1.5 | (0.625%) | 1.5 | (0.625%) | 1.5 | (0.625%) | 1.5 | (0.625%) |
| Micronized, 100% | | | | | | | | | | |
| Lactose | 129.9 | (54.125%) | 117.9 | (49.125%) | 129.9 | (54.125%) | 117.9 | (49.125%) | 93.9 | (39.125%) |
| Monohydrate, | | | | | | | | | | |
| Spray Dried, NF | | | | | | | | | | |
| Microcrystalline | 96 | (40%) | 96 | (40%) | 96 | (40%) | 96 | (40%) | 96 | (40%) |
| Cellulose, NF, | | | | | | | | | | |
| (Avicel PH 200) | | | | | | | | | | |
| HPMC K100M | 12 | (5%) | 24 | (10%) | 12 | (5%) | 24 | (10%) | 48 | (20%) |
| Premium CR | | | | | | | | | | |
| Magnesium | 0.6 | (0.25%) | 0.6 | (0.25%) | 0.6 | (0.25%) | 0.6 | (0.25%) | 0.6 | (0.25%) |
| Stearate, NF | | | | | | | | | | |

### Example 21

### Dissolution Profiles of Bi-Layer Tablets and Models thereof

To evaluate of the influence of HPMC levels in CE and MPA layers on the dissolution of MPA and CE, a modified distance response surface experimental design was employed to prepare model formulations. Nine model formulations were randomly generated by Design Expert® 6.0.9 software, corresponding to Examples 20A-20I.

The dissolution of CE for Examples 20A-20I was determined using USP Apparatus 2, at 50 rpm in 900 mL of 0.02M Sodium Acetate Buffer, pH 4.5 for a period of 8 hours. Filtered samples of the dissolution medium were taken at 1, 2, 3, 4 and 5 hr. The release of the active was determined on a reversed phase high performance liquid chromatography (HPLC). The results are shown in Table XXI and FIGS. 32 to 40.

The dissolution of MPA for Examples 20A-20I was determined using USP Apparatus 2, at 50 rpm in 900 mL with 0.54% sodium lauryl sulfate (SLS) in water for a period of 12 hours. A filtered sample of the dissolution medium was taken at 15, 30, 60, 120, and 360 minutes. The release of the active was determined by reversed phase high performance liquid chromatography (HPLC). The results are shown in Table XXII and FIGS. 17 to 25.

The CE released percentage at 1, 2, 3, 4 and 5 hr and the MPA released percentage at 15, 30, 60, 120, and 360 minutes were treated by Design Expert® 6.0.9 software and were fit using a quadratic model:

Quadratic model: Y=f+aA+bB+cA²+dB²+eAB
Note: A: HPMC level in CE layer
   B: HPMC level in MPA layer
      Table XXIII lists all the coefficients for optimal regression equation for CE dissolution, while Table XXIV displays the statistical parameters for MPA release rate.

Several batches with different levels of HPMC K100M CR in CE and MPA layers were manufactured (Examples 20J-20O). Dissolution rates of CE as well as MPA of these batches were determined. The actual values from these dissolution tests were compared with the predicted values from the experimental design. The results are shown in Table XXV and XXVI. The data indicate that the actual values are fairly close to the predicted values. FIGS. 26 to 31 show the dissolution profiles for MPA for Examples 20J to 20O, respectively. FIGS. 41 to 46 shown the dissolution profiles for CE for Examples 20J to 20O, respectively.

TABLE XXI

| | HPMC in | | | | | | | |
|---|---|---|---|---|---|---|---|---|
| | CE/MPA | Percent CE Released at Different Time Intervals (% ± sd, n = 6) | | | | | | |
| Run | Layer | 0 hr | 1 hr | 2 hr | 3 hr | 4 hr | 5 hr | 8 hr |
| Example 20A | 35/20 | 0 | 22 ± 2.5 | 33.4 ± 2.5 | 43 ± 2.1 | 50 ± 2.6 | 57 ± 2.5 | 73 ± 2.9 |
| Example 20B | 10/20 | 0 | 47 ± 3.5 | 62 ± 4.9 | 73 ± 3.8 | 81 ± 3.8 | 87 ± 3.8 | 95 ± 3.0 |
| Example 20C | 35/5 | 0 | 28 ± 1.9 | 48 ± 3.6 | 64 ± 4.1 | 75 ± 4.8 | 85 ± 4.3 | 97 ± 3.3 |
| Example 20D | 10/5 | 0 | 57 ± 9.6 | 78 ± 11.3 | 91 ± 9.7 | 97 ± 7.0 | 99 ± 4.6 | 101 ± 2.6 |
| Example 20E | 35/10 | 0 | 22 ± 2.3 | 36 ± 2.7 | 47 ± 2.9 | 56 ± 3.1 | 63 ± 3.8 | 79 ± 4.1 |
| Example 20F | 55/5 | 0 | 20 ± 2.1 | 34 ± 3.1 | 45 ± 3.7 | 54 ± 4.1 | 53 ± 4.3 | 81 ± 4.0 |
| Example 20G | 55/10 | 0 | 17 ± 0.8 | 28 ± 1.2 | 37 ± 1.1 | 44 ± 0.9 | 51 ± 1.0 | 66 ± 0.9 |
| Example 20H | 55/20 | 0 | 16 ± 0.6 | 25 ± 0.6 | 33 ± 0.7 | 40 ± 1.1 | 46 ± 1.1 | 61 ± 0.9 |
| Example 20I | 10/10 | 0 | 44 ± 4.8 | 63 ± 6.0 | 75 ± 6.1 | 85 ± 6.8 | 91 ± 7.1 | 98 ± 4.7 |

TABLE XXII

| | HPMC in | | | | | | | | |
|---|---|---|---|---|---|---|---|---|---|
| Example | CE/MPA | Percent MPA Released at Different Time Intervals (% ± sd, n = 6) | | | | | | | |
| # | Layer | 0 min | 15 min | 30 min | 45 min | 60 min | 120 min | 360 min | 720 min |
| 20A | 35/20 | 0 | 3 ± 0.3 | 4 ± 0.7 | 6 ± 0.9 | 7 ± 1.2 | 14 ± 2.8 | 43 ± 6.2 | 81 ± 7.6 |
| 20B | 10/20 | 0 | 7 ± 0.5 | 7 ± 0.4 | 8 ± 0.6 | 10 ± 1.0 | 20 ± 2.4 | 55 ± 4.0 | 100 ± 4.2 |
| 20C | 35/5 | 0 | 42 ± 21.7 | 52 ± 21.3 | 57 ± 20.2 | 62 ± 19.1 | 72 ± 17.2 | 89 ± 8.6 | 95 ± 5.0 |
| 20D | 10/5 | 0 | 48 ± 13.2 | 58 ± 13.3 | 66 ± 13.4 | 71 ± 12.8 | 86 ± 10.7 | 101 ± 4.8 | 104 ± 3.0 |
| 20E | 35/10 | 0 | 11 ± 3.6 | 16 ± 4.6 | 19 ± 4.9 | 22 ± 5.6 | 33 ± 6.8 | 68 ± 11.0 | 98 ± 4.1 |
| 20F | 55/5 | 0 | 25 ± 7.2 | 34 ± 9.9 | 41 ± 11.4 | 47 ± 13.2 | 63 ± 17.6 | 86 ± 12.0 | 97 ± 1.1 |
| 20G | 55/10 | 0 | 9 ± 0.5 | 13 ± 0.6 | 16 ± 1.0 | 19 ± 1.3 | 30 ± 3.0 | 67 ± 9.2 | 93 ± 6.2 |
| 20H | 55/20 | 0 | 2 ± 0.4 | 3 ± 0.5 | 4 ± 0.8 | 6 ± 1.1 | 13 ± 2.6 | 41 ± 6.5 | 75 ± 6.1 |
| Example | 10/10 | 0 | 12 ± 1.9 | 19 ± 2.4 | 24 ± 3.1 | 28 ± 3.8 | 43 ± 5.1 | 88 ± 5.2 | 106 ± 1.4 |
| 20I | | | | | | | | | |

TABLE XXIII

| Coeffi- | | | | | |
|---|---|---|---|---|---|
| cient | Y_{cE 1 h} | Y_{cE 2 h} | Y_{CE 3 h} | Y_{CE 4 h} | Y_{CE 5 h} |
| f | 84.405 | 112.029 | 128.469 | 133.525 | 133.182 |
| a*A | −1.801 | −1.825 | −1.687 | −1.437 | −1.064 |
| b*B | −3.141 | −4.401 | −5.218 | −5.053 | −4.893 |
| c*A² | 0.0159 | 0.0134 | 0.0105 | 0.00776 | 0.004363 |
| d*B² | 0.0991 | 0.131 | 0.153 | 0.152 | 0.1558 |
| e*A*B | 0.00609 | 0.00794 | 0.00741 | 0.000658 | −0.0076 |

TABLE XXIV

| Coefficient | Y_{MPA 15 min} | Y_{MPA 30 min} | Y_{MPA 60 min} | Y_{MPA 120 min} | Y_{MPA 360 min} |
|---|---|---|---|---|---|
| f | 94.7399 | 113.1339 | 133.966 | 153.718 | 133.7326 |
| a*A | −0.2561 | −0.2832 | −0.446 | −0.8427 | −1.134 |
| b*B | −10.7494 | −12.549 | −14.0527 | −14.196 | −4.458 |
| c*A² | −0.0038874 | −0.00428 | −0.0021667 | 0.003872 | 0.0115 |
| d*B² | 0.3088 | 0.35267 | 0.38816 | 0.38144 | 0.05789 |
| e*A*B | 0.02228 | 0.025698 | 0.02607 | 0.023435 | 0.0006761 |

TABLE XXV

| | HPMC % | HPMC % | | | | | | |
|---|---|---|---|---|---|---|---|---|
| | in CE | in MPA | | | | | | |
| Batch # | Layer | Layer | | Y_{CE 1 h} | Y_{CE 2 h} | Y_{CE 3 h} | Y_{CE 4 h} | Y_{CE 5 h} |
| Example 20J | 20 | 20 | Actual | 29 ± 2.4 | 43 ± 1.5 | 55 ± 2.4 | 65 ± 2.1 | 72 ± 2.4 |
| | | | Predict | 34 | 48 | 59 | 68 | 75 |
| Example 20K | 20 | 5 | Actual | 42 ± 6.9 | 63 ± 4.9 | 77 ± 3.6 | 88 ± 2.5 | 94 ± 1.9 |
| | | | Predict | 42 | 63 | 77 | 87 | 92 |
| Example 20L | 20 | 10 | Actual | 34 ± 9.6 | 50 ± 11.0 | 63 ± 12.1 | 73 ± 12.0 | 80 ± 13.0 |
| | | | Predict | 35 | 52 | 64 | 73 | 79 |
| Example 20M | 45 | 5 | Actual | 22 ± 1.8 | 36 ± 2.9 | 47 ± 3.8 | 56 ± 4.6 | 65 ± 5.3 |
| | | | Predict | 24 | 40 | 53 | 63 | 72 |
| Example 20N | 45 | 10 | Actual | 21 ± 1.4 | 33 ± 2.3 | 43 ± 3.1 | 52 ± 3.7 | 59 ± 4.2 |
| | | | Predict | 17 | 30 | 40 | 50 | 57 |
| Example 20O | 45 | 20 | Actual | 19 ± 2.2 | 30 ± 2.0 | 39 ± 1.8 | 45 ± 2.2 | 52 ± 2.8 |
| | | | Predict | 18 | 29 | 37 | 45 | 52 |

Note:
the Actual displayed is the percent released (%) ± sd, n = 6

TABLE XXVI

| | HPMC % | HPMC % | | | | | | |
|---|---|---|---|---|---|---|---|---|
| | in CE | in MPA | | | | | | |
| Batch # | Layer | Layer | | Y_{15 min} | Y_{30 min} | Y_{60 min} | Y_{120 min} | Y_{360 min} |
| Example 20J | 20 | 20 | Actual | 2 ± 0.4 | 3 ± 0.7 | 6 ± 1.6 | 13 ± 3.0 | 43 ± 6.6 |
| | | | Predict | 6 | 6 | 9 | 16 | 50 |
| Example 20K | 20 | 5 | Actual | 40 ± 14.0 | 52 ± 14.4 | 67 ± 14.8 | 81 ± 15.3 | 95 ± 3.4 |
| | | | Predict | 44 | 54 | 66 | 79 | 95 |
| Example 20L | 20 | 10 | Actual | 13 ± 1.7 | 18 ± 2.3 | 24 ± 3.2 | 36 ± 3.4 | 77 ± 4.4 |
| | | | Predict | 16 | 21 | 28 | 39 | 77 |
| Example 20M | 45 | 5 | Actual | 31 ± 12.5 | 40 ± 13.3 | 52 ± 13.0 | 67 ± 10.8 | 93 ± 3.0 |
| | | | Predict | 34 | 44 | 55 | 68 | 85 |
| Example 20N | 45 | 10 | Actual | 8 ± 1.5 | 12 ± 1.9 | 17 ± 2.4 | 27 ± 3.7 | 64 ± 11.1 |
| | | | Predict | 9 | 13 | 20 | 30 | 68 |
| Example 20O | 45 | 20 | Actual | 2 ± 0.2 | 3 ± 0.4 | 6 ± 0.9 | 14 ± 2.2 | 44 ± 6.0 |
| | | | Predict | 4 | 5 | 7 | 13 | 41 |

Note:
the Actual displayed is the percent released (%) ± sd, n = 6

### Example 22

### Bazedoxifene Acetate Granulation Containing 5.1% Hypomellose

Using the ingredients in the amounts shown in Table XXVII, bazedoxifene acetate (“BZA”) was granulated with the other ingredients using the procedure below for a batch size of 1 kg.

1. Avicel PH200 and BZA were passed through a #30 mesh screen.
2. These ingredient were blended together in a 4 Qt V-blender for approximately 5 minutes at about 22 rpm.
3. Lactose monohydrate and hypromellose were added into the blender and blended for approximately 15 minutes at about 22 rpm.
4. The magnesium stearate was screened with about 100 g of the blended material through the #30 mesh screen.
5. The mixture of step 4 was added into the blender and blend for approximately 3 minutes at about 22 rpm.
6. The blend of step 5 was granulated using Alexanderwerk WP 120×40 Roller
Compactor at following parameters:
Screen Feeder Speed: 55 rpm
Roller Speed: 7 rpm
Fine Granulator Speed: 60 rpm
Hydraulic Pressure: 40 bar
Roller Gap: 1.5 mm
Vacuum: on

TABLE XXVII

| Ingredients | mg/tab | w/w % |
|---|---|---|
| Bazedoxifene acetate, Micronized (A) | 22.58 | 7.53 |
| Lactose monohydrate spray dried | 138.85 | 46.28 |
| Microcrystalline cellulose, Avicel PH 200 | 122.49 | 40.83 |
| Hypromellose K100M Prem CR | 15.31 | 5.10 |
| Magnesium Stearate | 0.77 | 0.26 |

Note:
(A) dosed as free base. Quantity is adjusted based on the actual potency.

### Example 23

### Bazedoxifene Acetate Granulation Containing 10.21% Hypomellose

Using the ingredients in the amounts shown in Table XXVIII, bazedoxifene acetate was granulated with the other ingredients by following the procedure of Example 22.

TABLE XXVIII

| Ingredients | mg/tab | w/w % |
|---|---|---|
| Bazedoxifene acetate, Micronized (A) | 22.58 | 7.53 |
| Lactose monohydrate spray dried | 123.54 | 41.18 |
| Microcrystalline cellulose, Avicel PH 200 | 122.49 | 40.83 |
| Hypromellose K100M Prem CR | 30.62 | 10.21 |
| Magnesium stearate | 0.77 | 0.26 |

Note:
(A) dosed as free base. Quantity is adjusted based on the actual potency.

### Example 24

### Bazedoxifene Acetate Granulation Containing 20.41% Hypomellose

Using the ingredients in the amounts shown in Table XXIX, bazedoxifene acetate was granulated with the other ingredients by following the procedure of Example 22.

TABLE XXIX

| Ingredients | mg/tab | w/w % |
|---|---|---|
| Bazedoxifene acetate, Micronized (A) | 22.58 | 7.53 |
| Lactose monohydrate spray dried | 92.92 | 30.97 |
| Microcrystalline cellulose, Avicel PH 200 | 122.49 | 40.83 |
| Hypromellose K100M Prem CR | 61.24 | 20.41 |
| Magnesium Stearate | 0.77 | 0.26 |

Note:
(A) dosed as free base. Quantity is adjusted based on the actual potency.

### Example 25

### Bazedoxifene Acetate Granulation Containing 5% Hypomellose and Antioxidants

Using the ingredients in the amounts shown in Table XXX, bazedoxifene acetate was granulated with the other ingredients using the procedure below for a batch size of 1 kg:

1. Avicel PH200 and BZA were passed through a #30 mesh screen.
2. The ingredients were blended together in a 4 Qt V-blender for approximately 5 minutes at about 22 rpm.
3. The lactose and hypromellose were added into the blender and blend for approximately 15 minutes at about 22 rpm.
4. The intra-granular magnesium stearate was screened with about 100 g of the blended material through the #30 mesh screen.
5. The mixture in Step 4 was added into the blender and blend for approximately 3 minutes at about 22 rpm.
6. The step 2 blend was granulated using Fitzpatrick Chilsonator IR 220 at following parameters:
Roll Pressure: approximately 90-210 psi
Roll Force: approximately 500-700 lb/in
Roll Speed: approximately 9 rpm
VFS: approximately 150-200 rpm
HFS: approximately 50-60 rpm
   7. The ribbon was milled using a Quadro Comil 197S at about 20% motor speed using screen with about 1.575 mm opening.
   8. The milled materials were then weighed.
   9. The milled materials were blended in the 4 Qt V-blender for approximately 10 minutes at about 22 rpm.
   10. The quantity of extra-granular magnesium stearate needed was calculated based on the yield was then calculated.
   11. The magnesium stearate was weighed and add to the blender and blend for approximately 3 minutes at about 22 rpm.

TABLE XXX

| Ingredients | mg/tab | w/w % |
|---|---|---|
| Bazedoxifene acetate, Micronized (A) | 22.58 | 7.53 |
| Lactose monohydrate spray dried | 135.97 | 45.32 |
| Microcystalline cellulose, Avicel PH 200 | 120 | 40.00 |
| Hypromellose K100M Prem CR | 15 | 5.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |

Note:
(A) dosed as free base. Quantity is adjusted based on the actual potency.

### Example 26

### Bazedoxifene Acetate Granulation Containing 10% Hypomellose and Antioxidants

Using the ingredients in the amounts shown in Table XXXI, bazedoxifene acetate was granulated with the other ingredients by following the procedure of Example 25.

TABLE XXXI

| Ingredients | mg/tab | w/w % |
|---|---|---|
| Bazedoxifene acetate, Micronized (A) | 22.58 | 7.53 |
| Lactose monohydrate spray dried | 120.97 | 40.32 |
| Microcrystalline cellulose, Avicel PH 200 | 120 | 40.00 |
| Hypromellose K100M Prem CR | 30 | 10.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

### Example 27

### Bazedoxifene Acetate Granulation Containing 20% Hypomellose and Antioxidants

Using the ingredients in the amounts shown in Table XXXII, bazedoxifene acetate was granulated with the other ingredients by following the procedure of Example 25.

TABLE XXXII

| Ingredients | mg/tab | w/w % |
|---|---|---|
| Bazedoxifene acetate, Micronized (A) | 22.58 | 7.53 |
| Lactose monohydrate spray dried | 90.97 | 30.32 |
| Microcrystalline cellulose, Avicel PH 200 | 120 | 40.00 |
| Hypromellose K100M Prem CR | 60 | 20.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |

Note:
(A) dosed as free base. Quantity is adjusted based on the actual potency.

### Example 28A

### Bi-Layer Tablet Prepared from CE Granulation and BZA Granulation

Using the CE granulation of Example 1 and the BZ granulation of Example 22, a CE/BZA bi-layer tablet was compressed using a Kilian RUD compression machine with 11 mm round convex tooling. The targeted total bi-layer tablet weight was 420 mg with 300 mg and 120 mg for the BZA and CE sub-layer portion, respectively. The compression force was adjusted in order to get bi-layer tablets within the targeted hardness range of 15-19 kp. Under this compression force the bi-layer tablet had a friability of zero percent.

### Example 28B

### Bi-Layer Tablet Prepared from CE Granulation and BZA Granulation

Using the CE granulation of Example 1 and the BZ granulation of Example 23, bi-layer tablets were prepared according to the procedure of Example 28A.

### Example 28C

### Bi-Layer Tablet Prepared from CE Granulation and BZA Granulation

Using the CE granulation of Example 1 and the BZ granulation of Example 24, bi-layer tablets were prepared according to the procedure of Example 28A.

### Example 29

### Weight Variation of CE/BZA Bi-Layer Tablets

Weight variation of 100 tablets was evaluated using the Mocon Automatic Balance Analysis testers. The results are shown in Table XXXIII.

TABLE XXXIII

| Batch # | Weight Variation (%) |
|---|---|
| Example 28A (5% HPMC K100M CR in | 0.72 |
| BZA Layer; 27.5% HPMC K100M CR in | |
| CE layer) | |
| Example 28B (10% HPMC K100M CR in | 1.15 |
| BZA Layer; 27.5% HPMC K100M CR in | |
| CE layer) | |
| Example 28C (20% HPMC K100M CR in | 1.18 |
| BZA Layer; 27.5% HPMC K100M CR in | |
| CE layer) | |

### Example 30

### Dissolution of Bazedoxifene Acetate from Bi-Layer Tablets

The dissolution of BZA from the bi-layer tablets of Examples 28A-28C was determined using USP Apparatus 1 (basket), at 75 rpm in 900 mL of 10 mM acetate acid solution with 0.2% polysorbate 80 (Tween 80) at 37° C.±0.5° C. for a period of 60 minutes. Then the speed changed to 250 rpm for data point at 80 minutes. A filtered sample of the dissolution medium was taken at specified time intervals. The release of the active was determined by reversed phase high performance liquid chromatography (HPLC). The results are shown in Table XXXIV and FIGS. 47 to 49 for Examples 28A to 28C, respectively.

TABLE XXXIV

| | Percent Dissolved (% ± sd, n = 3) | | |
|---|---|---|---|
| Time (minute) | Example 28A | Example 28B | Example 28C |
| 0 | 0 | 0 | 0 |
| 20 | 14 ± 3.5 | 4 ± 0.2 | 3 ± 0.3 |
| 40 | 20 ± 3.5 | 6 ± 0.3 | 5 ± 0.3 |
| 60 | 25 ± 3.4 | 8 ± 0.5 | 6 ± 0.3 |
| 80 | 37 ± 3.9 | 11 ± 0.6 | 8 ± 0.1 |

### Example 31

### Dissolution of Conjugated Estrogens from Bi-Layer Tablets

The dissolution of CE for Examples 28A-28C was determined using USP Apparatus 2, at 50 rpm in 900 mL of 0.02M Sodium Acetate Buffer, pH 4.5 for a period of 8 hours. Filtered samples of the dissolution medium were taken at specified time intervals. The release of the active was determined on a reversed phase high performance liquid chromatography (HPLC). The results are shown in Table XXXV and FIGS. 50 to 52 for Examples 28A to 28C, respectively.

TABLE XXXV

| | Percent Dissolved (% ± sd, n = 6) | | |
|---|---|---|---|
| Time (hr) | Example 28A | Example 28B | Example 28C |
| 0 | 0 | 0 | 0 |
| 1 | 31.06 ± 3.2 | 23.48 ± 2.4 | 23.23 ± 2.1 |
| 2 | 51.45 ± 4.6 | 37.43 ± 2.3 | 35.73 ± 1.4 |
| 3 | 66.42 ± 5.2 | 46.52 ± 2.1 | 45.31 ± 2.0 |
| 5 | 83.55 ± 6.8 | 60.21 ± 2.4 | 58.54 ± 1.9 |
| 8 | 94.75 ± 6.7 | 72.23 ± 2.4 | 70.82 ± 1.9 |

### Example 32

### Conjugated Estrogens/Bazedoxifene (BZA) (0.45 MG/20 MG) Modified Release Bi-Layer Formulation

This Example illustrates a CE/bazedoxifene (BZA) (0.45 mg/20 mg) bi-layer formulations.

### 1. Materials and Methods

A. Formulation and Manufacturing Procedures for BZA with Different Levels/Viscosity Grades of HPMC and Antioxidants Dry Granulation

The compositions of BZA with different levels/viscosity grades of HPMC and antioxidants, ascorbic acid and dl-α-tocopheryl acetate, granulation are listed in Tables XXXVI-XLII (1-7).

TABLE XXXVI (1)
Composition of BZA Granulation with 5% HPMC
K100M CR and Antioxidants Formulation D

| Ingredients | mg/tab | w/w % |
|---|---|---|
| BZA Micronized (A) | 22.58 | 7.53 |
| Lactose monohydrate spray dried | 135.97 | 45.32 |
| Avicel PH 200 | 120 | 40.00 |
| Hypromellose K100M Prem CR | 15 | 5.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

TABLE XXXVII 2
Composition of BZA Granulation with 10% HPMC
K100M CR and Antioxidants (Formulation E)

| Ingredients | mg/tab | w/w % |
|---|---|---|
| BZA Micronized (A) | 22.58 | 7.53 |
| Lactose monohydrate spray dried | 120.97 | 40.32 |
| Avicel PH 200 | 120 | 40.00 |
| Hypromellose K100M Prem CR | 30 | 10.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

TABLE XXXVIII 3
Composition of BZA Granulation with 20% HPMC
K100M CR and Antioxidants (Formulation F)

| Ingredients | mg/tab | w/w % |
|---|---|---|
| BZA Micronized (A) | 22.58 | 7.53 |
| Lactose monohydrate spray dried | 90.97 | 30.32 |
| Avicel PH 200 | 120 | 40.00 |
| Hypromellose K100M Prem CR | 60 | 20.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

TABLE XXXIX 4
Composition of BZA Granulation with 5% HPMC
K100 LV and Antioxidants (Formulation G)

| Ingredients | mg/tab | w/w % |
|---|---|---|
| BZA Micronized (A) | 22.58 | 7.53 |
| Lactose monohydrate spray dried | 135.97 | 45.32 |
| Avicel PH 200 | 120 | 40.00 |
| Hypromellose K100 LV | 15 | 5.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

TABLE XL 5
Composition of BZA Granulation with 20% HPMC
K100 LV and Antioxidants (Formulation H)

| Ingredients | mg/tab | w/w % |
|---|---|---|
| BZA Micronized (A) | 22.58 | 7.53 |
| Lactose monohydrate spray dried | 90.97 | 30.32 |
| Avicel PH 200 | 120 | 40.00 |
| Hypromellose K100 LV | 60 | 20.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

TABLE XL1 6
Composition of BZA Granulation with 5% HPMC
K4M CR and Antioxidants (Formulation I)

| Ingredients | mg/tab | w/w % |
|---|---|---|
| BZA Micronized (A) | 22.58 | 7.53 |
| Lactose monohydrate spray dried | 135.97 | 45.32 |
| Avicel PH 200 | 120 | 40.00 |
| Hypromellose K4M Prem CR | 15 | 5.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

TABLE XLII 7
Composition of BZA Granulation with 20% HPMC
K4M CR and Antioxidants (Formulation J)

| Ingredients | mg/tab | w/w % |
|---|---|---|
| BZA Micronized (A) | 22.58 | 7.53 |
| Lactose monohydrate spray dried | 90.97 | 30.32 |
| Avicel PH 200 | 120 | 40.00 |
| Hypromellose K4M Prem CR | 60 | 20.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

The dry granulation was completed using a Fitzpatrick Chilsonator IR 220 using the procedures below for a batch size of 1 kg:
1. Pass Avicel PH200 and BZA through a #30 mesh screen.
2. Blend them together in a 4 Qt V-blender for approximately 5 minutes at about 22 rpm.
3. Add lactose and hypromellose into the blender and blend for approximately 15 minutes at about 22 rpm.
4. Screen the intra-granular magnesium stearate with about 100 g of the blended material through the #30 mesh screen.
5. Add the mixture in Step 4 into the blender and blend for approximately 3 minutes at about 22 rpm.
6. Granulate step 2 blend using Fitzpatrick Chilsonator IR 220 at following parameters:
Roll Pressure: approximately 90-210 psi
Roll Force: approximately 500-2500 lb/in
Roll Speed: approximately 9 rpm
VFS: approximately 150-200 rpm
HFS: approximately 50-60 rpm
7. Mill the ribbon using a Quadro Comil 197S at about 20% motor speed using screen with about 1.575 mm opening.
8. Weigh the milled materials.
9. Blend the milled materials in the 4 Qt V-blender for approximately 10 minutes at about 22 rpm.
10. Calculate the quantity of extra-granular MS needed based on the yield.
11. Weigh the MS and add to the blender and blend for approximately 3 minutes at about 22 rpm.

B. Formulation and Manufacturing Procedures for CE Layer Granulation

The composition of CE with 27.5% HPMC K100M granulation is listed in Table XLIII. A CEDL at 42.9 mg/g mixture was granulated with all other ingredients using water in a high shear granulator followed by the procedures below for a batch size of 1.5 kg:

1. Mix CEDL with Lactose Spray Dried, Avicel®, and HPMC in a Collette shear mixer for approximately 5 minutes with plows at approximately 430 rpm.

2. Granulate the Step #1 blend by initiating the addition of water with plows and choppers set at approximately 430 and 1800 rpm, respectively. Add all the water within approximately 4 minutes.

3. Continue granulation for total of approximately 7 minutes.

4. Dry the wet granulation in a fluid bed dryer at an inlet temperature set point of 60° C. to achieve a target granulation LOD of 2%. A variation of ±0.5% moisture content is acceptable.

5. Pass the dried granulation through a Model “M” Fitzmill equipped with a # 2A plate, set at a high speed (4500-4600 rpm), and impact set forward.

6. Mix the granulation of Step 5 in a V-Blender for approximately 10 minutes at approximately 22 rpm.

7. Remove about 100 g of Step 6 blend for use in Step 8.

8. Add the Magnesium Stearate (MS) through a # 20 screen, in approximately equal portions, to each side of the V-blender. After the MS addition, add the Step 7 blend, in approximately equal portions, to each side of V-Blender. Blend for approximately 3 minutes. The quantity of MS added must be adjusted on a per tablet basis based on the quantity of granulation to be blended.

9. Discharge the Step 8 lubricated granulation into a double-bagged polyethylene bag with a desiccant bag in between the bags.

TABLE XLIII
Composition of Conjugated Estrogens Internal
Tablet Portion with 27.5% HPMC K100M CR

| | Input/Tablet | |
|---|---|---|
| Description | (mg) | % W/W |
| CE Desiccation with Lactose at 42.9 mg/g | 10.4895 | 8.74 |
| Lactose Monohydrate Spray Dried | 58.2105 | 48.51 |
| Avicel PH 101, NF | 18 | 15.00 |
| HPMC K100M Premium CR | 33 | 27.5 |
| Magnesium Stearate, NF | 0.3 | 0.25 |
| Purified Water, USP (A) | 30 | |

Note:
(A) Indicates removed during processing.

C. Compression of CE/BZA Bi-Layer Tablets

The CE/BZA bi-layer tablet was compressed using a Kilian RUD compression machine with 11 mm round convex tooling. The targeted total bi-layer tablet weight was 420 mg with 300 mg and 120 mg for the BZA and CE sub-layer portion, respectively. The compression force was adjusted in order to get bi-layer tablets within the targeted hardness range of 15-19 kp. Under this compression force the bi-layer tablet had a friability of zero percent. The correspondent batch numbers of these bi-layer tablets are as following:

TABLE XLIV
Correspondent Batch Numbers of Bi-layer
Tablet Versus Granulation

| Granulation Batch Number | Bi-layer Tablet Batch Number |
|---|---|
| L34419-52 | L34419-55 |
| L34419-53 | L34419-56 |
| L34419-54 | L34419-67 |
| L34419-58 | L34419-62 |
| L34419-59 | L34419-63 |
| L34419-60 | L34419-64 |
| L34419-61 | L34419-65 |

### 2. Results and Discussion

A. Dissolution Profiles of CE and BZA from Bi-Layer Tablet Formulations D to J

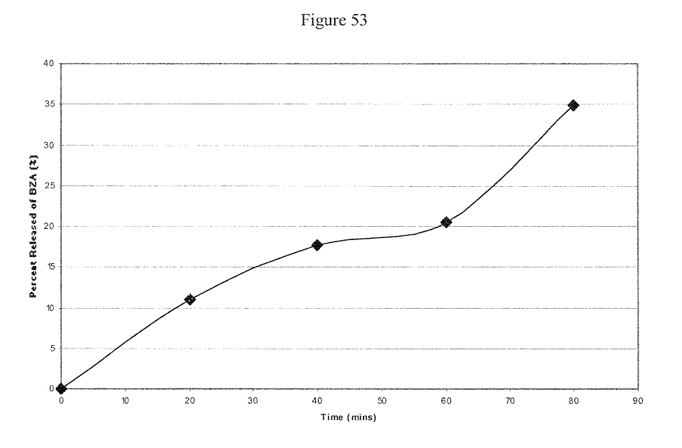

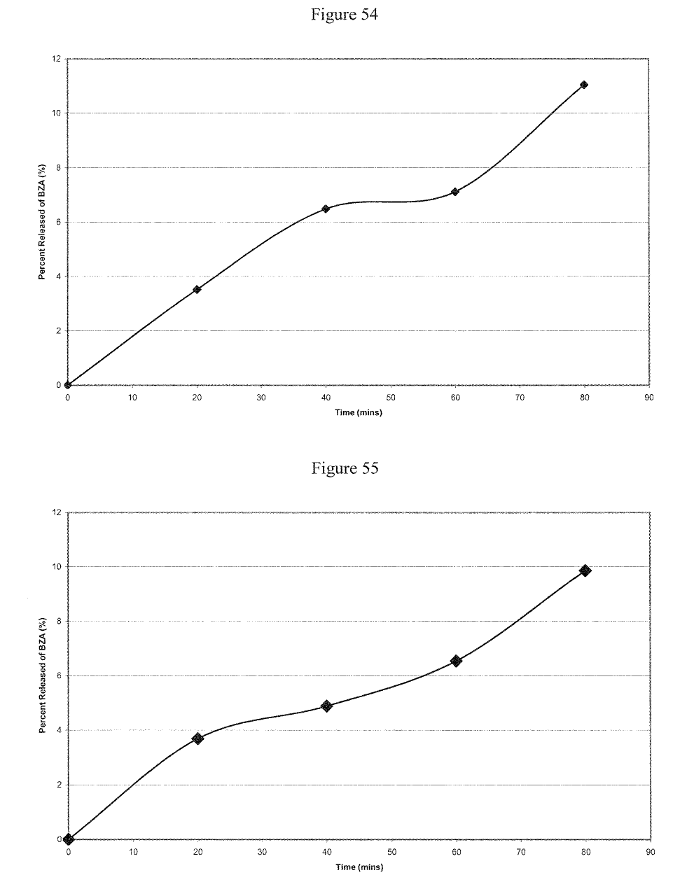

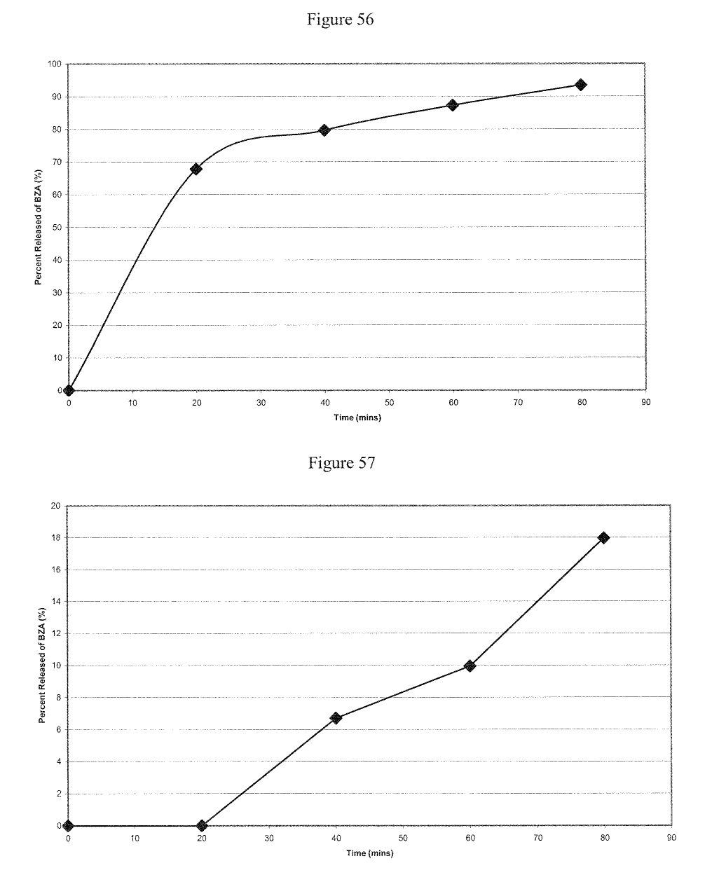

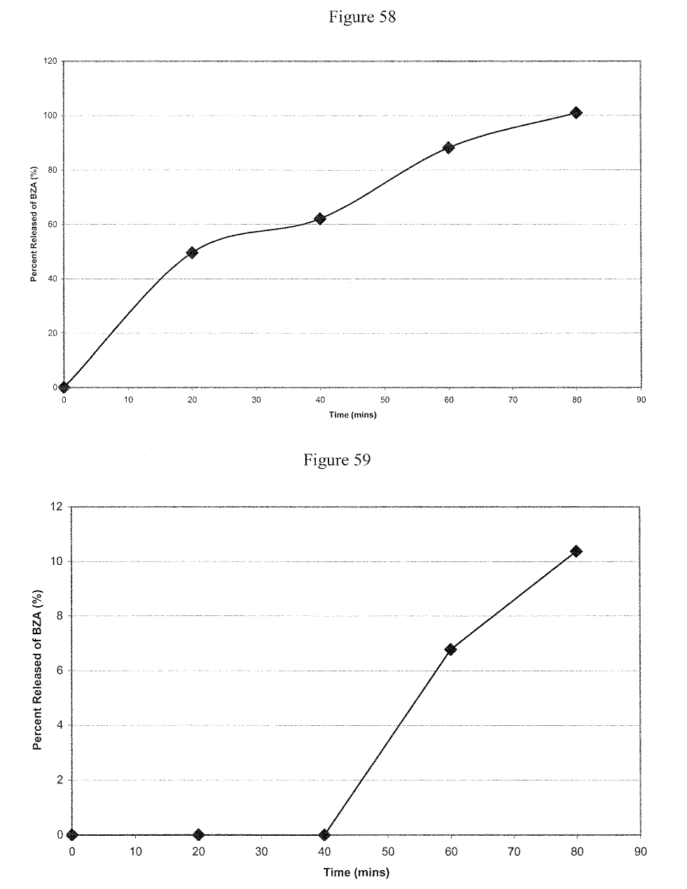

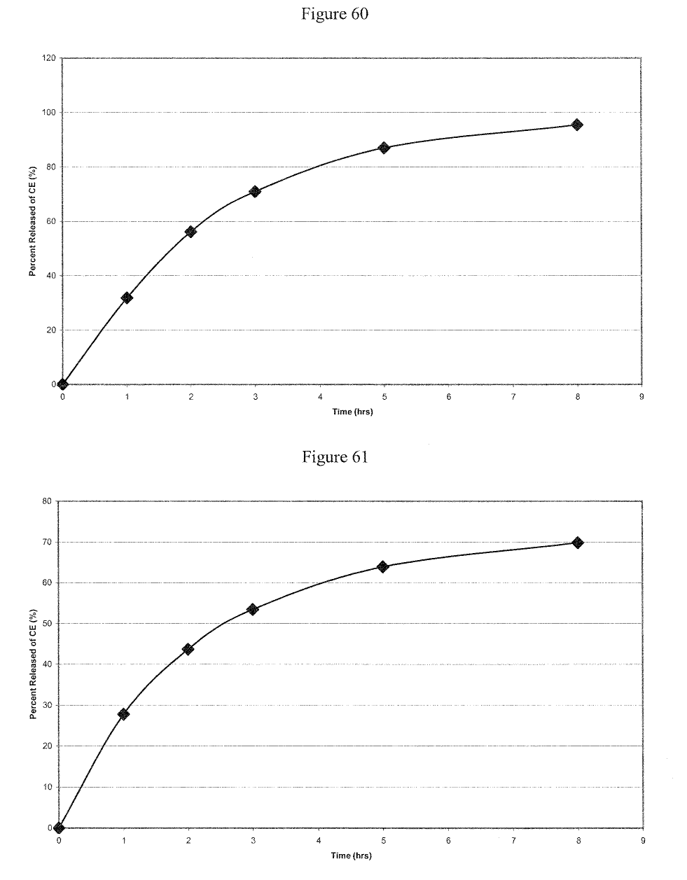

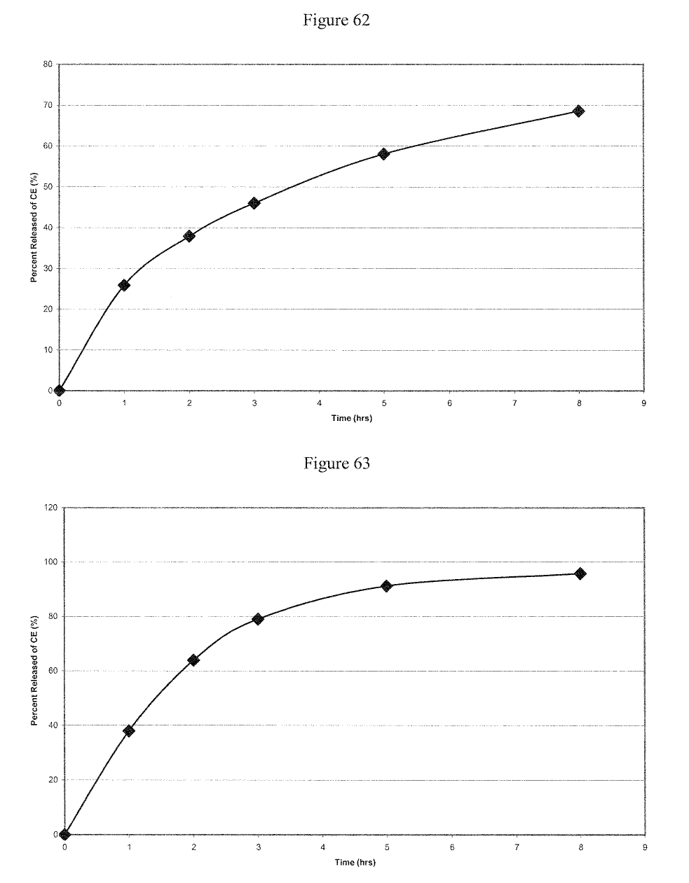

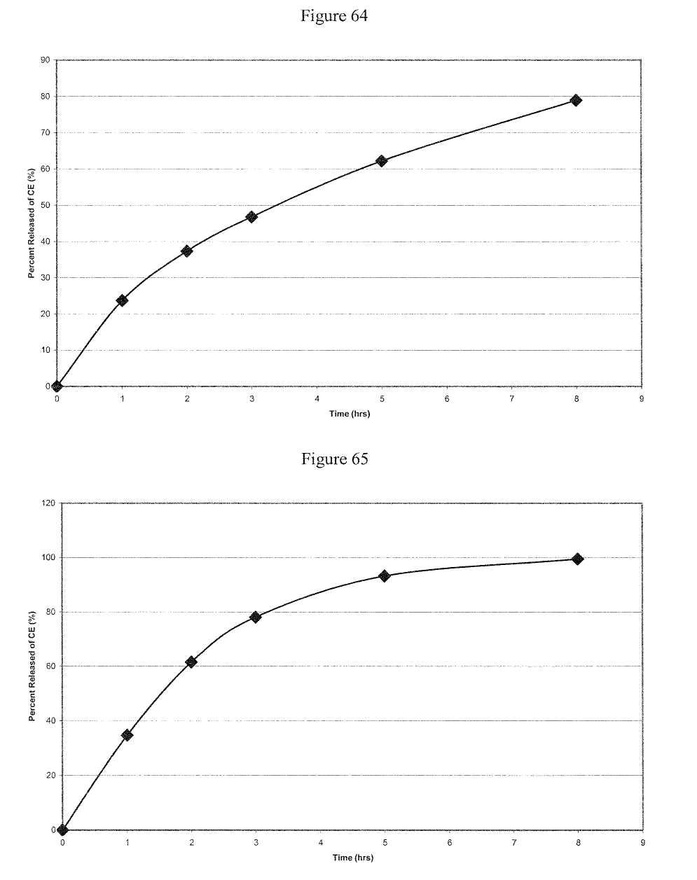

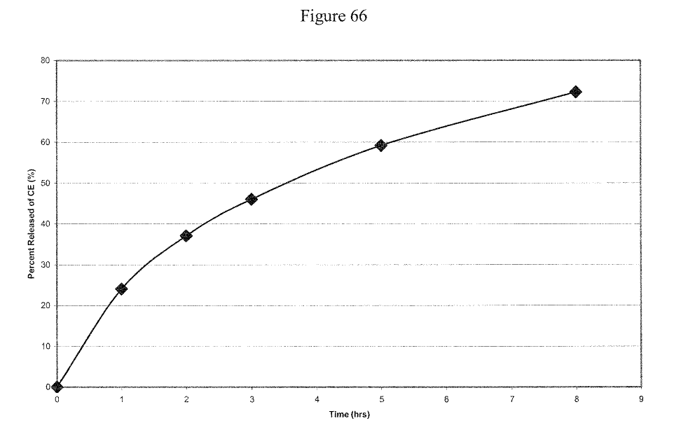

The dissolution profiles of BZA and CE from formulation D to J are listed in Tables XLV, XLVI, and FIGS. 53 to 66. From the results, it can be seen that a high level of polymer in one layer will slow down the dissolution rates of both layers. In addition, at same level, a higher viscosity grade of polymer in one layer will retard the dissolution rate of both layers.

TABLE XLV
Dissolution of BZA from CE/BZA Bi-layer Tablets

| | Percent Dissolved (% ± sd, n = 3) | | | | | | |
|---|---|---|---|---|---|---|---|
| Time | L34419- | L34419- | L34419- | L34419- | L34419- | L34419- | L34419- |
| (min) | 55 | 56 | 57 | 62 | 63 | 64 | 65 |
| 0 | 0 | 0 | 0 | 0 | 0 | 0 | 0 |
| 20 | 11 ± 2.6 | 4 ± 2.1 | 4 ± 0.6 | 68 ± 22.7 | 0 | 50 ± 5.8 | 0 |
| 40 | 18 ± 3.7 | 6 ± 2.7 | 5 ± 0.7 | 80 ± 15.0 | 7 ± 1.2 | 62 ± 8.2 | 0 |
| 60 | 21 ± 2.6 | 7 ± 2.8 | 7 ± 0.8 | 87 ± 8.7 | 10 ± 1.7 | 88 ± 8.0 | 7 ± 0.5 |
| 80 | 35 ± 4.6 | 11 ± 2.1 | 10 ± 0.7 | 93 ± 1.3 | 18 ± 3.1 | 101 ± 0.3 | 10 ± 0.5 |

TABLE XLVI
Dissolution of CE from CE/BZA Tablet-in-tablets

| | Percent Dissolved (% ± sd, n = 6) | | | | | | |
|---|---|---|---|---|---|---|---|
| Time | L34419- | L34419- | L34419- | L34419- | L34419- | L34419- | L34419- |
| (hr) | 55 | 56 | 57 | 62 | 63 | 64 | 65 |
| 0 | 0 | 0 | 0 | 0 | 0 | 0 | 0 |
| 1 | 32 ± 1.8 | 28 ± 2.4 | 26 ± 2.0 | 38 ± 8.1 | 24 ± 2.3 | 35 ± 5.6 | 24 ± 3.3 |
| 2 | 56 ± 2.5 | 43 ± 3.7 | 38 ± 2.3 | 64 ± 11.4 | 37 ± 2.4 | 62 ± 8.2 | 37 ± 3.2 |
| 3 | 71 ± 1.5 | 53 ± 4.0 | 46 ± 2.4 | 79 ± 11.7 | 47 ± 2.8 | 78 ± 8.7 | 46 ± 3.1 |
| 5 | 87 ± 3.7 | 64 ± 4.1 | 58 ± 2.3 | 91 ± 9.6 | 62 ± 2.6 | 93 ± 6.3 | 59 ± 3.0 |
| 8 | 95 ± 5.1 | 70 ± 3.0 | 69 ± 2.1 | 96 ± 5.6 | 79 ± 3.1 | 99 ± 3.3 | 72 ± 3.7 |

Various modifications of the invention, in addition to those described herein, will be apparent to those skill in the art from the foregoing description. Such modifications are also intended to fall into the scope of the claims. Each reference cited in the present application, including patents, published applications, and journal articles, is incorporated herein by reference in its entirety.

What is claimed:

1. A bi-layer tablet comprising:
(a) a first layer comprising at least one estrogen; and
(b) a second layer comprising one or more therapeutic agents selected from the group consisting of a selective estrogen receptor modulator and a progestational agent.

2. The tablet of claim 1 wherein:
said first layer comprises from about 20% to about 45% by weight of said tablet; and
said second layer comprises from about 55% to about 80% by weight of said tablet.

3. The tablet of claim 1 wherein said estrogen comprises conjugated estrogens.

4. The tablet of claim 1 wherein said second layer comprises one or more therapeutic agents selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

5. The tablet of claim 4 wherein said therapeutic agent is 1-[4-(2-azepan-1-yl-ethoxy)-benzyl]-2-(4-hydroxy-phenyl)-3-methyl-1H-indol-5-ol, acetic acid salt.

6. The tablet of claim 1 wherein:
said estrogen comprises conjugated estrogens; and
said second layer comprises one or more therapeutic agents selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

7. The tablet of claim 1 wherein said first layer and said second layer each further independently comprise a hydrophilic gel-forming polymer component.

8. The tablet of claim 7 wherein:
said estrogen comprises conjugated estrogens; and
said second layer further comprises one or more therapeutic agents selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

9. The tablet of claim 8 wherein said hydrophilic gel-forming polymer component of said first layer and said hydrophilic gel-forming polymer component of said second layer each independently comprises one or more of hydroxypropylmethylcellulose, polyethylene oxide, hydroxypropylcellulose, hydroxyethylcellulose, methylcellulose, polyvinylpyrrolidone, xanthan gum, and guar gum.

10. The tablet of claim 8 wherein said hydrophilic gel-forming polymer component of said first layer and said hydrophilic gel-forming polymer component of said second layer each comprise hydroxypropylmethylcellulose.

11. The tablet of claim 7 wherein said hydrophilic gel-forming polymer component of said first layer and said hydrophilic gel-forming polymer component of said second layer each independently comprises one or more of hydroxypropylmethylcellulose, polyethylene oxide, hydroxypropylcellulose, hydroxyethylcellulose, methylcellulose, polyvinylpyrrolidone, xanthan gum, and guar gum.

12. The tablet of claim 7 wherein said hydrophilic gel-forming polymer component of said first layer and said hydrophilic gel-forming polymer component of said second layer each comprise hydroxypropylmethylcellulose.

13. The tablet of claim 7 wherein:
said hydrophilic gel-forming polymer component of said first layer comprises from about 5% to about 80% by weight of said first layer; and
said hydrophilic gel-forming polymer component of said second layer comprises from about 1% to about 40% by weight of said second layer.

14. The tablet of claim 13 wherein said hydrophilic gel-forming polymer component of said first layer and said hydrophilic gel-forming polymer component of said second layer each independently comprises one or more of hydroxypropylmethylcellulose, polyethylene oxide, hydroxypropylcellulose, hydroxyethylcellulose, methylcellulose, polyvinylpyrrolidone, xanthan gum, and guar gum.

15. The tablet of claim 13 wherein said hydrophilic gel-forming polymer component of said first layer and said hydrophilic gel-forming polymer component of said second layer each independently comprises hydroxpropylmethylcellulose.

16. The tablet of claim 13 wherein:
(a) said first layer further comprises:
a filler/diluent component comprising from about 10% to about 90% by weight of said first layer;
a filler/binder component comprising from about 0.1% to about 30% by weight of said first layer; and
optionally, a lubricant component comprising from about 0.01% to about 3% by weight of said first layer; and
(b) said second layer further comprises:
a filler/diluent component comprising from about 10% to about 75% by weight of said second layer;
a filler/binder component comprising up to about 60% by weight of said second layer;
optionally, a lubricant component comprising from about 0.01% to about 3% by weight of said second layer;
optionally, a disintegrant comprising up to about 4% by weight of said second layer; and
optionally, an antioxidant component comprising from about 0.01% to about 4% by weight of said second layer.

17. The tablet of claim 16 wherein:
said filler/diluent component of said first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said first layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer component of said first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said filler/diluent component of said second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said second layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer of said second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional disintegrant of said second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose; and
said optional antioxidant component of said second layer, if present, comprises one or more of ascorbic acid, sodium ascorbate, ascorbyl palmitate, vitamin E, vitamin E acetate, butylated hydroxytoluene, and butylated hydroxyanisole.

18. The tablet of claim 16 wherein
said filler/diluent component of said first layer comprises one or more of lactose or lactose monohydrate;
said filler/binder component of said first layer comprises microcrystalline cellulose;
said hydrophilic gel-forming polymer component of said first layer comprises hydroxypropylmethylcellulose;
said optional lubricant component of said second layer, if present, comprises magnesium stearate;
said filler/diluent component of said second layer comprises one or more of lactose or lactose monohydrate;
said filler/binder component of said second layer comprises microcrystalline cellulose;
said hydrophilic gel-forming polymer component of said second layer comprises hydroxypropylmethylcellulose;
said optional lubricant component of said second layer, if present, comprises magnesium stearate;
said optional disintegrant of said second layer, if present, comprises croscarmellose sodium; and
said optional antioxidant component of said second layer, if present, comprises one or more of ascorbic acid and vitamin E acetate.

19. The tablet of claim 7 wherein:
said hydrophilic gel-forming polymer component of said first layer comprises from about 30% to about 40% by weight of said first layer; and
said hydrophilic gel-forming polymer component of said second layer comprises from about 15% to about 30% by weight of said second layer.

20. The tablet of claim 19 wherein:
(a) said first layer further comprises:
a filler/diluent component comprising from about 30% to about 70% by weight of said first layer;
a filler/binder component comprising from about 5% to about 25% by weight of said first layer; and
optionally, a lubricant component comprising from about 0.01% to about 2% by weight of said first layer; and
(b) said second layer further comprises:
a filler/diluent component comprising from about 25% to about 50% by weight of said second layer;
a filler/binder component comprising from about 20% to about 60% by weight of said second layer;
optionally, a lubricant component comprising from about 0.01% to about 2% by weight of said second layer;
optionally, a disintegrant comprising up to about 4% by weight of said second layer; and
optionally, an antioxidant component comprising from about 0.01% to about 4% by weight of said second layer.

21. The tablet of claim 20 wherein:
said filler/diluent component of said first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said first layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer component of said first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said filler/diluent component of said second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said second layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer of said second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional disintegrant of said second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
said optional antioxidant component of said second layer, if present, comprises one or more of ascorbic acid, sodium ascorbate, ascorbyl palmitate, vitamin E, vitamin E acetate, butylated hydroxytoluene, and butylated hydroxyanisole;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

22. The tablet of claim 20 wherein:
said filler/diluent component of said first layer comprises one or more of lactose or lactose monohydrate;
said filler/binder component of said first layer comprises microcrystalline cellulose;
said hydrophilic gel-forming polymer component of said first layer comprises hydroxypropylmethylcellulose;
said optional lubricant component of said first layer, if present, comprises magnesium stearate;
said filler/diluent component of said second layer comprises one or more of lactose or lactose monohydrate;
said filler/binder component of said second layer comprises microcrystalline cellulose;
said hydrophilic gel-forming polymer component of said second layer comprises hydroxypropylmethylcellulose;
said optional lubricant component of said second layer, if present, comprises magnesium stearate;
said optional disintegrant of said second layer, if present, comprises croscarmellose sodium;
said optional antioxidant component of said second layer, if present, comprises one or more of ascorbic acid and vitamin E acetate;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

23. The tablet of claim 7 wherein:
said hydrophilic gel-forming polymer component of said first layer comprises from about 5% to about 15% by weight of said first layer; and
said hydrophilic gel-forming polymer component of said second layer comprises from about 15% to about 30% by weight of said second layer.

24. The tablet of claim 23 wherein:
(a) said first layer further comprises:
a filler/diluent component comprising from about 50% to about 85% by weight of said first layer;
a filler/binder component comprising from about 5% to about 25% by weight of said first layer; and
optionally, a lubricant component comprising from about 0.01% to about 2% by weight of said first layer; and
(b) said second layer further comprises:
a filler/diluent component comprising from about 20% to about 60% by weight of said second layer;
a filler/binder component comprising from about 20% to about 60% by weight of said second layer;
optionally, a lubricant component comprising from about 0.01% to about 2% by weight of said second layer;
optionally, a disintegrant comprising up to about 4% by weight of said second layer; and
optionally, an antioxidant component comprising from about 0.01% to about 4% by weight of said second layer.

25. The tablet of claim 24 wherein:
said filler/diluent component of said first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said first layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer component of said first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said filler/diluent component of said second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said second layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer of said second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional disintegrant of said second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
said optional antioxidant component of said second layer, if present, comprises one or more of ascorbic acid, sodium ascorbate, ascorbyl palmitate, vitamin E, vitamin E acetate, butylated hydroxytoluene, and butylated hydroxyanisole;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

26. The tablet of claim 24 wherein:
said filler/diluent component of said first layer comprises one or more of lactose or lactose monohydrate;
said filler/binder component of said first layer comprises microcrystalline cellulose;
said hydrophilic gel-forming polymer component of said first layer comprises hydroxypropylmethylcellulose;
said optional lubricant component of said first layer, if present, comprises magnesium stearate;
said filler/diluent component of said second layer comprises one or more of lactose or lactose monohydrate;
said filler/binder component of said second layer comprises microcrystalline cellulose;
said hydrophilic gel-forming polymer component of said second layer comprises hydroxypropylmethylcellulose;
said optional lubricant component of said second layer, if present, comprises magnesium stearate;
said optional disintegrant of said second layer, if present, comprises croscarmellose sodium;
said optional antioxidant component of said second layer, if present, comprises one or more of ascorbic acid and vitamin E acetate;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

27. The tablet of claim 7 wherein:
said hydrophilic gel-forming polymer component of said first layer comprises from about 30% to about 40% by weight of said first layer; and
said hydrophilic gel-forming polymer component of said second layer comprises from about 1% to about 8% by weight of said second layer.

28. The tablet of claim 27 wherein:
(a) said first layer further comprises:
a filler/diluent component comprising from about 30% to about 70% by weight of said first layer;
a filler/binder component comprising from about 5% to about 25% by weight of said first layer; and
optionally, a lubricant component comprising from about 0.01% to about 2% by weight of said first layer; and
(b) said second layer further comprises:
a filler/diluent component comprising from about 35% to about 75% by weight of said second layer;
a filler/binder component comprising from about 20% to about 60% by weight of said second layer;
optionally, a lubricant component comprising from about 0.01% to about 2% by weight of said second layer;
optionally, a disintegrant comprising up to about 4% by weight of said second layer; and
optionally, an antioxidant component comprising from about 0.01% to about 4% by weight of said second layer.

29. The tablet of claim 28 wherein:
said filler/diluent component of said first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said first layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer component of said first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said filler/diluent component of said second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said second layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer of said second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional disintegrant of said second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
said optional antioxidant component of said second layer, if present, comprises one or more of ascorbic acid, sodium ascorbate, ascorbyl palmitate, vitamin E, vitamin E acetate, butylated hydroxytoluene, and butylated hydroxyanisole;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

30. The tablet of claim 28 wherein:
said filler/diluent component of said first layer comprises one or more of lactose or lactose monohydrate;
said filler/binder component of said first layer comprises microcrystalline cellulose;
said hydrophilic gel-forming polymer component of said first layer comprises hydroxypropylmethylcellulose;
said optional lubricant component of said first layer, if present, comprises magnesium stearate;
said filler/diluent component of said second layer comprises one or more of lactose or lactose monohydrate;
said filler/binder component of said second layer comprises microcrystalline cellulose;
said hydrophilic gel-forming polymer component of said second layer comprises hydroxypropylmethylcellulose;
said optional lubricant component of said second layer, if present, comprises magnesium stearate;
said optional disintegrant of said second layer, if present, comprises croscarmellose sodium;
said optional antioxidant component of said second layer, if present, comprises one or more of ascorbic acid and vitamin E acetate;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

31. The tablet of claim 7 wherein:
said hydrophilic gel-forming polymer component of said first layer comprises from about 5% to about 15% by weight of said first layer; and
said hydrophilic gel-forming polymer component of said second layer comprises from about 1% to about 8% by weight of said second layer.

32. The tablet of claim 31 wherein:
(a) said first layer further comprises:
a filler/diluent component comprising from about 50% to about 85% by weight of said first layer;
a filler/binder component comprising from about 5% to about 25% by weight of said first layer; and
optionally, a lubricant component comprising from about 0.01% to about 2% by weight of said first layer; and
(b) said second layer further comprises:
a filler/diluent component comprising from about 35% to about 75% by weight of said second layer;
a filler/binder component comprising from about 20% to about 60% by weight of said second layer;
optionally, a lubricant component comprising from about 0.01% to about 2% by weight of said second layer;
optionally, a disintegrant comprising up to about 4% by weight of said second layer; and
optionally, an antioxidant component comprising from about 0.01% to about 4% by weight of said second layer.

33. The tablet of claim 32 wherein:
said filler/diluent component of said first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said first layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer component of said first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said filler/diluent component of said second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said second layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer of said second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional disintegrant of said second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
said optional antioxidant component of said second layer, if present, comprises one or more of ascorbic acid, sodium ascorbate, ascorbyl palmitate, vitamin E, vitamin E acetate, butylated hydroxytoluene, and butylated hydroxyanisole;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

34. The tablet of claim 32 wherein:
said filler/diluent component of said first layer comprises one or more of lactose or lactose monohydrate;
said filler/binder component of said first layer comprises microcrystalline cellulose;
said hydrophilic gel-forming polymer component of said first layer comprises hydroxypropylmethylcellulose;
said optional lubricant component of said first layer, if present, comprises magnesium stearate;
said filler/diluent component of said second layer comprises one or more of lactose or lactose monohydrate;
said filler/binder component of said second layer comprises microcrystalline cellulose;
said hydrophilic gel-forming polymer component of said second layer comprises hydroxypropylmethylcellulose;
said optional lubricant component of said second layer, if present, comprises magnesium stearate;
said optional disintegrant of said second layer, if present, comprises croscarmellose sodium;
said optional antioxidant component of said second layer, if present, comprises one or more of ascorbic acid and vitamin E acetate;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

35. The tablet of claim 7 wherein:
said hydrophilic gel-forming polymer component of said first layer comprises from about 30% to about 40% by weight of said first layer; and
said hydrophilic gel-forming polymer component of said second layer comprises from about 8% to about 15% by weight of said second layer.

36. The tablet of claim 35 wherein:
(a) said first layer further comprises:
a filler/diluent component comprising from about 30% to about 70% by weight of said first layer;
a filler/binder component comprising from about 5% to about 25% by weight of said first layer; and
optionally, a lubricant component comprising from about 0.01% to about 2% by weight of said first layer; and
(b) said second layer further comprises:
a filler/diluent component comprising from about 35% to about 70% by weight of said second layer;
a filler/binder component comprising from about 20% to about 60% by weight of said second layer;
optionally, a lubricant component comprising from about 0.01% to about 2% by weight of said second layer;
optionally, a disintegrant comprising up to about 4% by weight of said second layer; and
optionally, an antioxidant component comprising from about 0.01% to about 4% by weight of said second layer.

37. The tablet of claim 36 wherein:
said filler/diluent component of said first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said first layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer component of said first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said filler/diluent component of said second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said second layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer of said second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional disintegrant of said second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
said optional antioxidant component of said second layer, if present, comprises one or more of ascorbic acid, sodium ascorbate, ascorbyl palmitate, vitamin E, vitamin E acetate, butylated hydroxytoluene, and butylated hydroxyanisole;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

38. The tablet of claim 36 wherein:
said filler/diluent component of said first layer comprises one or more of lactose or lactose monohydrate;
said filler/binder component of said first layer comprises microcrystalline cellulose;
said hydrophilic gel-forming polymer component of said first layer comprises hydroxypropylmethylcellulose;
said optional lubricant component of said first layer, if present, comprises magnesium stearate;
said filler/diluent component of said second layer comprises one or more of lactose or lactose monohydrate;
said filler/binder component of said second layer comprises microcrystalline cellulose;
said hydrophilic gel-forming polymer component of said second layer comprises hydroxypropylmethylcellulose;
said optional lubricant component of said second layer, if present, comprises magnesium stearate;
said optional disintegrant of said second layer, if present, comprises croscarmellose sodium;
said optional antioxidant component of said second layer, if present, comprises one or more of ascorbic acid and vitamin E acetate;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

39. The tablet of claim 7 wherein:
said hydrophilic gel-forming polymer component of said first layer comprises from about 40% to about 60% by weight of said first layer; and
said hydrophilic gel-forming polymer component of said second layer comprises from about 1% to about 8% by weight of said second layer.

40. The tablet of claim 39 wherein:
(a) said first layer further comprises:
a filler/diluent component comprising from about 10% to about 50% by weight of said first layer;
a filler/binder component comprising from about 5% to about 25% by weight of said first layer; and
optionally, a lubricant component comprising from about 0.01% to about 2% by weight of said first layer; and
(b) said second layer further comprises:
a filler/diluent component comprising from about 35% to about 75% by weight of said second layer;
a filler/binder component comprising from about 20% to about 60% by weight of said second layer;
optionally, a lubricant component comprising from about 0.01% to about 2% by weight of said second layer;
optionally, a disintegrant comprising up to about 4% by weight of said second layer; and
optionally, an antioxidant component comprising from about 0.01% to about 4% by weight of said second layer.

41. The tablet of claim 40 wherein:
said filler/diluent component of said first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said first layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer component of said first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said filler/diluent component of said second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said second layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer of said second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional disintegrant of said second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
said optional antioxidant component of said second layer, if present, comprises one or more of ascorbic acid, sodium ascorbate, ascorbyl palmitate, vitamin E, vitamin E acetate, butylated hydroxytoluene, and butylated hydroxyanisole;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

42. The tablet of claim 40 wherein:
said filler/diluent component of said first layer comprises one or more of lactose or lactose monohydrate;
said filler/binder component of said first layer comprises microcrystalline cellulose;
said hydrophilic gel-forming polymer component of said first layer comprises hydroxypropylmethylcellulose;
said optional lubricant component of said first layer, if present, comprises magnesium stearate;
said filler/diluent component of said second layer comprises one or more of lactose or lactose monohydrate;
said filler/binder component of said second layer comprises microcrystalline cellulose;
said hydrophilic gel-forming polymer component of said second layer comprises hydroxypropylmethylcellulose;
said optional lubricant component of said second layer, if present, comprises magnesium stearate;
said optional disintegrant of said second layer, if present, comprises croscarmellose sodium;
said optional antioxidant component of said second layer, if present, comprises one or more of ascorbic acid and vitamin E acetate;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

43. The tablet of claim 7 wherein:
said hydrophilic gel-forming polymer component of said first layer comprises from about 40% to about 60% by weight of said first layer; and
said hydrophilic gel-forming polymer component of said second layer comprises from about 8% to about 15% by weight of said second layer.

44. The tablet of claim 43 wherein:
(a) said first layer further comprises:
a filler/diluent component comprising from about 10% to about 50% by weight of said first layer;
a filler/binder component comprising from about 5% to about 25% by weight of said first layer; and
optionally, a lubricant component comprising from about 0.01% to about 2% by weight of said first layer; and
(b) said second layer further comprises:
a filler/diluent component comprising from about 35% to about 70% by weight of said second layer;
a filler/binder component comprising from about 20% to about 60% by weight of said second layer;
optionally, a lubricant component comprising from about 0.01% to about 2% by weight of said second layer;
optionally, a disintegrant comprising up to about 4% by weight of said second layer; and
optionally, an antioxidant component comprising from about 0.01% to about 4% by weight of said second layer.

45. The tablet of claim 44 wherein:
said filler/diluent component of said first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said first layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer component of said first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said filler/diluent component of said second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said second layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer of said second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional disintegrant of said second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
said optional antioxidant component of said second layer, if present, comprises one or more of ascorbic acid, sodium ascorbate, ascorbyl palmitate, vitamin E, vitamin E acetate, butylated hydroxytoluene, and butylated hydroxyanisole;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

46. The tablet of claim 44 wherein:
said filler/diluent component of said first layer comprises one or more of lactose or lactose monohydrate;
said filler/binder component of said first layer comprises microcrystalline cellulose;
said hydrophilic gel-forming polymer component of said first layer comprises hydroxypropylmethylcellulose;
said optional lubricant component of said first layer, if present, comprises magnesium stearate;
said filler/diluent component of said second layer comprises one or more of lactose or lactose monohydrate;
said filler/binder component of said second layer comprises microcrystalline cellulose;
said hydrophilic gel-forming polymer component of said second layer comprises hydroxypropylmethylcellulose;
said optional lubricant component of said second layer, if present, comprises magnesium stearate;
said optional disintegrant of said second layer, if present, comprises croscarmellose sodium;
said optional antioxidant component of said second layer, if present, comprises one or more of ascorbic acid and vitamin E acetate;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

47. The tablet of claim 7 wherein:
said hydrophilic gel-forming polymer component of said first layer comprises from about 40% to about 60% by weight of said first layer; and
said hydrophilic gel-forming polymer component of said second layer comprises from about 15% to about 30% by weight of said second layer.

48. The tablet of claim 47 wherein:
(a) said first layer further comprises:
a filler/diluent component comprising from about 10% to about 50% by weight of said first layer;
a filler/binder component comprising from about 5% to about 25% by weight of said first layer; and
optionally, a lubricant component comprising from about 0.01% to about 2% by weight of said first layer; and
(b) said second layer further comprises:
a filler/diluent component comprising from about 20% to about 60% by weight of said second layer;
a filler/binder component comprising from about 20% to about 60% by weight of said second layer;
optionally, a lubricant component comprising from about 0.01% to about 2% by weight of said second layer;
optionally, a disintegrant comprising up to about 4% by weight of said second layer; and
optionally, an antioxidant component comprising from about 0.01% to about 4% by weight of said second layer.

49. The tablet of claim 48 wherein:
said filler/diluent component of said first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said first layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer component of said first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said filler/diluent component of said second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said second layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer of said second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional disintegrant of said second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
said optional antioxidant component of said second layer, if present, comprises one or more of ascorbic acid, sodium ascorbate, ascorbyl palmitate, vitamin E, vitamin E acetate, butylated hydroxytoluene, and butylated hydroxyanisole;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

50. The tablet of claim 48 wherein:
said filler/diluent component of said first layer comprises one or more of lactose or lactose monohydrate;
said filler/binder component of said first layer comprises microcrystalline cellulose;
said hydrophilic gel-forming polymer component of said first layer comprises hydroxypropylmethylcellulose;
said optional lubricant component of said first layer, if present, comprises magnesium stearate;
said filler/diluent component of said second layer comprises one or more of lactose or lactose monohydrate;
said filler/binder component of said second layer comprises microcrystalline cellulose;
said hydrophilic gel-forming polymer component of said second layer comprises hydroxypropylmethylcellulose;
said optional lubricant component of said second layer, if present, comprises magnesium stearate;
said optional disintegrant of said second layer, if present, comprises croscarmellose sodium;
said optional antioxidant component of said second layer, if present, comprises one or more of ascorbic acid and vitamin E acetate;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

51. The tablet of claim 7 wherein:
said hydrophilic gel-forming polymer component of said first layer comprises from about 5% to about 15% by weight of said first layer; and
said hydrophilic gel-forming polymer component of said second layer comprises from about 8% to about 15% by weight of said second layer.

52. The tablet of claim 51 wherein:
(a) said first layer further comprises:
a filler/diluent component comprising from about 50% to about 85% by weight of said first layer;
a filler/binder component comprising from about 5% to about 25% by weight of said first layer; and
optionally, a lubricant component comprising from about 0.01% to about 2% by weight of said first layer; and
(b) said second layer further comprises:
a filler/diluent component comprising from about 35% to about 70% by weight of said second layer;
a filler/binder component comprising from about 20% to about 60% by weight of said second layer;
optionally, a lubricant component comprising from about 0.01% to about 2% by weight of said second layer;
optionally, a disintegrant comprising up to about 4% by weight of said second layer; and
optionally, an antioxidant component comprising from about 0.01% to about 4% by weight of said second layer.

53. The tablet of claim 52 wherein:
said filler/diluent component of said first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said first layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer component of said first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said filler/diluent component of said second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said second layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer of said second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional disintegrant of said second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
said optional antioxidant component of said second layer, if present, comprises one or more of ascorbic acid, sodium ascorbate, ascorbyl palmitate, vitamin E, vitamin E acetate, butylated hydroxytoluene, and butylated hydroxyanisole;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

54. The tablet of claim 52 wherein:
said filler/diluent component of said first layer comprises one or more of lactose or lactose monohydrate;
said filler/binder component of said first layer comprises microcrystalline cellulose;
said hydrophilic gel-forming polymer component of said first layer comprises hydroxypropylmethylcellulose;
said optional lubricant component of said first layer, if present, comprises magnesium stearate;
said filler/diluent component of said second layer comprises one or more of lactose or lactose monohydrate;
said filler/binder component of said second layer comprises microcrystalline cellulose;
said hydrophilic gel-forming polymer component of said second layer comprises hydroxypropylmethylcellulose;
said optional lubricant component of said second layer, if present, comprises magnesium stearate;
said optional disintegrant of said second layer, if present, comprises croscarmellose sodium;
said optional antioxidant component of said second layer, if present, comprises one or more of ascorbic acid and vitamin E acetate;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

55. The tablet of claim 7 wherein:
said hydrophilic gel-forming polymer component of said first layer comprises from about 15% to about 30% by weight of said first layer; and
said hydrophilic gel-forming polymer component of said second layer comprises from about 15% to about 30% by weight of said second layer.

56. The tablet of claim 55 wherein:
(a) said first layer further comprises:
a filler/diluent component comprising from about 40% to about 80% by weight of said first layer;
a filler/binder component comprising from about 5% to about 25% by weight of said first layer; and
optionally, a lubricant component comprising from about 0.01% to about 2% by weight of said first layer; and
(b) said second layer further comprises:
a filler/diluent component comprising from about 0.0% to about 60% by weight of said second layer;
a filler/binder component comprising from about 20% to about 60% by weight of said second layer; and
optionally, a lubricant component comprising from about 0.01% to about 2% by weight of said second layer;
optionally, a disintegrant comprising up to about 4% by weight of said second layer; and
optionally, an antioxidant component comprising from about 0.01% to about 4% by weight of said second layer.

57. The tablet of claim 56 wherein:
said filler/diluent component of said first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said first layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer component of said first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said filler/diluent component of said second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said second layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer of said second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional disintegrant of said second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
said optional antioxidant component of said second layer, if present, comprises one or more of ascorbic acid, sodium ascorbate, ascorbyl palmitate, vitamin E, vitamin E acetate, butylated hydroxytoluene, and butylated hydroxyanisole;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

58. The tablet of claim 56 wherein:
said filler/diluent component of said first layer comprises one or more of lactose or lactose monohydrate;
said filler/binder component of said first layer comprises microcrystalline cellulose;
said hydrophilic gel-forming polymer component of said first layer comprises hydroxypropylmethylcellulose;
said optional lubricant component of said first layer, if present, comprises magnesium stearate;
said filler/diluent component of said second layer comprises one or more of lactose or lactose monohydrate;
said filler/binder component of said second layer comprises microcrystalline cellulose;
said hydrophilic gel-forming polymer component of said second layer comprises hydroxypropylmethylcellulose;
said optional lubricant component of said second layer, if present, comprises magnesium stearate;
said optional disintegrant of said second layer, if present, comprises croscarmellose sodium;
said optional antioxidant component of said second layer, if present, comprises one or more of ascorbic acid and vitamin E acetate;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

59. The tablet of claim 7 wherein:
said hydrophilic gel-forming polymer component of said first layer comprises from about 15% to about 30% by weight of said first layer; and
said hydrophilic gel-forming polymer component of said second layer comprises from about 1% to about 8% by weight of said second layer.

60. The tablet of claim 59 wherein:
(a) said first layer further comprises:
a filler/diluent component comprising from about 40% to about 80% by weight of said first layer;
a filler/binder component comprising from about 5% to about 25% by weight of said first layer; and
optionally, a lubricant component comprising from about 0.01% to about 2% by weight of said first layer; and
(b) said second layer further comprises:
a filler/diluent component comprising from about 35% to about 75% by weight of said second layer;
a filler/binder component comprising from about 20% to about 60% by weight of said second layer;
optionally, a lubricant component comprising from about 0.01% to about 2% by weight of said second layer;
optionally, a disintegrant comprising up to about 4% by weight of said second layer; and
optionally, an antioxidant component comprising from about 0.01% to about 4% by weight of said second layer.

61. The tablet of claim 60 wherein:
said filler/diluent component of said first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said first layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer component of said first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said filler/diluent component of said second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said second layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer of said second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional disintegrant of said second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
said optional antioxidant component of said second layer, if present, comprises one or more of ascorbic acid, sodium ascorbate, ascorbyl palmitate, vitamin E, vitamin E acetate, butylated hydroxytoluene, and butylated hydroxyanisole;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

62. The tablet of claim 60 wherein:
said filler/diluent component of said first layer comprises one or more of lactose or lactose monohydrate;
said filler/binder component of said first layer comprises microcrystalline cellulose;
said hydrophilic gel-forming polymer component of said first layer comprises hydroxypropylmethylcellulose;
said optional lubricant component of said first layer, if present, comprises magnesium stearate;
said filler/diluent component of said second layer comprises one or more of lactose or lactose monohydrate;
said filler/binder component of said second layer comprises microcrystalline cellulose;
said hydrophilic gel-forming polymer component of said second layer comprises hydroxypropylmethylcellulose;
said optional lubricant component of said second layer, if present, comprises magnesium stearate;
said optional disintegrant of said second layer, if present, comprises croscarmellose sodium;
said optional antioxidant component of said second layer, if present, comprises one or more of ascorbic acid and vitamin E acetate;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

63. The tablet of claim 7 wherein:
said hydrophilic gel-forming polymer component of said first layer comprises from about 15% to about 30% by weight of said first layer; and
said hydrophilic gel-forming polymer component of said second layer comprises from about 8% to about 15% by weight of said second layer.

64. The tablet of claim 63 wherein
(a) said first layer further comprises:
a filler/diluent component comprising from about 40% to about 80% by weight of said first layer;
a filler/binder component comprising from about 5% to about 25% by weight of said first layer; and
optionally, a lubricant component comprising from about 0.01% to about 2% by weight of said first layer; and
(b) said second layer further comprises:
a filler/diluent component comprising from about 35% to about 70% by weight of said second layer;
a filler/binder component comprising from about 20% to about 60% by weight of said second layer;
optionally, a lubricant component comprising from about 0.01% to about 2% by weight of said second layer;
optionally, a disintegrant comprising up to about 4% by weight of said second layer; and
optionally, an antioxidant component comprising from about 0.01% to about 4% by weight of said second layer.

65. The tablet of claim 64 wherein:
said filler/diluent component of said first layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said first layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer component of said first layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said first layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said filler/diluent component of said second layer comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said second layer comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer of said second layer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said second layer, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional disintegrant of said second layer, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
said optional antioxidant component of said second layer, if present, comprises one or more of ascorbic acid, sodium ascorbate, ascorbyl palmitate, vitamin E, vitamin E acetate, butylated hydroxytoluene, and butylated hydroxyanisole;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

66. The tablet of claim 64 wherein:
said filler/diluent component of said first layer comprises one or more of lactose or lactose monohydrate;
said filler/binder component of said first layer comprises microcrystalline cellulose;
said hydrophilic gel-forming polymer component of said first layer comprises hydroxypropylmethylcellulose;
said optional lubricant component of said first layer, if present, comprises magnesium stearate;
said filler/diluent component of said second layer comprises one or more of lactose or lactose monohydrate;
said filler/binder component of said second layer comprises microcrystalline cellulose;
said hydrophilic gel-forming polymer component of said second layer comprises hydroxypropylmethylcellulose;
said optional lubricant component of said second layer, if present, comprises magnesium stearate;
said optional disintegrant of said second layer, if present, comprises croscarmellose sodium;
said optional antioxidant component of said second layer, if present, comprises one or more of ascorbic acid and vitamin E acetate;
said estrogen comprises conjugated estrogens; and
said one or more therapeutic agents are selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

67. A tablet selected from a plurality of tablets according to claim 7, wherein the plurality has a mean dissolution profile wherein:
the mean of % of the estrogen released per tablet after 1, 2, 3, 4, and 5 hours under estrogen dissolution conditions is substantially equal to the sum of f, a*A, b*B, c*A², d*B², and e*A*B; and
the mean of % of the therapeutic agent per tablet released after 0.25, 0.5, 1, 2, and 6 hours under type I therapeutic agent dissolution conditions is substantially equal to the sum of m, n*A, o*B, p*A², q*B², and r*A*B;
wherein:
A is the % of hydrophilic gel-forming polymer by weight of said first layer;
B is the % of hydrophilic gel-forming polymer by weight of said second layer;
f, at 1 hour, is 84.405;
a, at 1 hour, is −1.801;
b, at 1 hour, is −3.141;
c, at 1 hour, is 0.0159;
d, at 1 hour, is 0.0991;
e, at 1 hour, is 0.00609;
f, at 2 hours, is 112.029;
a, at 2 hours, is −1.825;
b, at 2 hours, is −4.401;
c, at 2 hours, is 0.0134;
d, at 2 hours, is 0.131;
e, at 2 hours, is 0.00794;
f, at 3 hours, is 128.469;
a, at 3 hours, is −1.687;
b, at 3 hours, is −5.218;
c, at 3 hours, is 0.0105;
d, at 3 hours, is 0.153;
e, at 3 hours, is 0.00741;
f, at 4 hours, is 133.525;
a, at 4 hours, is −1.437;
b, at 4 hours, is −5.053;
c, at 4 hours, is 0.00776;
d, at 4 hours, is 0.152;
e, at 4 hours, is 0.000658;
f, at 5 hours, is 133.182;
a, at 5 hours, is −1.064;
b, at 5 hours, is −4.893;
c, at 5 hours, is 0.004363;
d, at 5 hours, is 0.1558;
e, at 5 hours, is −0.0076;
m, at 0.25 hour, is 94.7399;
n, at 0.25 hour, is −0.2561;
o, at 0.25 hour, is −10.7494;
p, at 0.25 hour, is −0.0038874;
q, at 0.25 hour, is 0.3088;
r, at 0.25 hour, is 0.02228;
m, at 0.5 hour, is 113.1339;
n, at 0.5 hour, is −0.2832;
o, at 0.5 hour, is −12.549;
p, at 0.5 hour, is −0.00428;
q, at 0.5 hour, is 0.35267;
r, at 0.5 hour, is 0.025698;
m, at 1 hour, is 133.966;
n, at 1 hour, is −0.446;
o, at 1 hour, is −14.0527;
p, at 1 hour, is −0.0021667;
q, at 1 hour, is 0.38816;
r, at 1 hour, is 0.02607;
m, at 2 hours, is 153.718;
n, at 2 hours, is −0.8427;
o, at 2 hours, is −14.196;
p, at 2 hours, is 0.003872;
q, at 2 hours, is 0.38144;
r, at 2 hours, is 0.023435;
m, at 6 hours, is 133.7326;
n, at 6 hours, is −1.134;
o, at 6 hours, is −4.458;
p, at 6 hours, is 0.0115;
q, at 6 hours, is 0.05789; and
r, at 6 hours, is 0.0006761.

68. The tablet according to claim 67 wherein:
said hydrophilic gel-forming polymer component of said first layer comprises from about 5% to about 80% by weight of said first layer; and
said hydrophilic gel-forming polymer component of said second layer comprises from about 1% to about 40% by weight of said second layer.

69. The tablet of claim 1 wherein:
said estrogen comprises conjugated estrogens;
said therapeutic agent comprises medroxyprogesterone acetate;
said dissolution profile of said estrogen from said tablet under estrogen dissolution conditions is substantially as shown in any one of FIGS. 9 to 16 and 32 to 46; and
said dissolution profile of said therapeutic agent from said tablet under type I therapeutic agent dissolution conditions is substantially as shown in any one of FIGS. 1 to 8 and 17 to 31.

70. The tablet of claim 1 wherein:
said estrogen comprises conjugated estrogens;
said therapeutic agent comprises 1-[4-(2-azepan-1-yl-ethoxy)-benzyl]-2-(4-hydroxy-phenyl)-3-methyl-1H-indol-5-ol, acetic acid salt;
said dissolution profile of said estrogen from said tablet under estrogen dissolution conditions is substantially as shown in any one of FIGS. 50 to 52; and
said dissolution profile of said therapeutic agent from said tablet under type II therapeutic agent dissolution conditions is substantially as shown in any one of FIGS. 47 to 49.

71. A process for producing a bi-layer tablet of claim 1 comprising compressing together:
a first mixture comprising at least one estrogen; and
a second mixture comprising one or more therapeutic agents selected from the group consisting of a selective estrogen receptor modulator and a progestational agent.

72. The process of claim 71 wherein:
said estrogen comprises conjugated estrogens; and
said second mixture comprises one or more therapeutic agents selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

73. The process of claim 71 wherein said first mixture is prepared by a process comprising the steps of:
(i) mixing said estrogen and a hydrophilic gel-forming polymer component to form an initial mixture;
(ii) granulating said initial mixture to form a first granulated mixture; and
(iii) milling said first granulated mixture.

74. The process of claim 73 wherein step (i) further comprises mixing said estrogen and said hydrophilic gel-forming polymer component with a filler/diluent component and a filler/binder component.

75. The process of claim 74 wherein step (ii) further comprises the steps of:
(x) adding water to said initial mixture during said granulating; and
(y) drying said first granulated mixture before said milling.

76. The process of claim 75 wherein said drying comprises drying said first granulated mixture to loss on drying (LOD) of from about 1% to about 3%.

77. The process of claim 74 wherein said second mixture is prepared by a process comprising blending said therapeutic agent and a hydrophilic gel-forming polymer component.

78. The process of claim 77 wherein said blending of said therapeutic agent and said hydrophilic gel-forming polymer further comprises blending with a filler/diluent component, a filler/binder component, optionally, an antioxidant component and, optionally, a disintegrant.

79. The process of claim 78 further comprising granulating said second mixture after said blending of said therapeutic agent, said hydrophilic gel-forming polymer filler/diluent component, said filler/binder component, said optional antioxidant component and said optional disintegrant.

80. The process of claim 79 further comprising:
blending said first mixture with a lubricant after said milling; and
blending said second mixture with a lubricant after said blending of said therapeutic agent, said filler/diluent component, said filler/binder component, said hydrophilic gel-forming polymer component, said optional antioxidant component, if present, and said optional disintegrant component, if present.

81. A product of the process of claim 80.

82. The process of claim 71 wherein:
(a) said first mixture further comprises:
a filler/diluent component comprising from about 10% to about 90% by weight of said first mixture;
a filler/binder component comprising from about 0.1% to about 30% by weight of said first mixture;
a hydrophilic gel-forming polymer component comprising from about 5% to about 80% by weight of said first mixture; and
optionally, a lubricant component comprising from about 0.01% to about 3% by weight of said first mixture; and
(b) said second mixture further comprises:
a filler/diluent component comprising from about 10% to about 75% by weight of said second mixture;
a filler/binder component comprising up to about 60% by weight of said second mixture;
a hydrophilic gel-forming polymer component comprising from about 1% to about 40% by weight of said second mixture;
optionally, a lubricant component comprising from about 0.01% to about 3% by weight of said second mixture;
optionally, a disintegrant comprising up to about 4% by weight of said second mixture; and
optionally, an antioxidant component comprising from about 0.01% to about 4% by weight of said second mixture.

83. The process of claim 82 wherein:
said filler/diluent component of said first mixture comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said first mixture comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer component of said first mixture comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said first mixture, if present comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said filler/diluent component of said second mixture comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said filler/binder component of said second mixture comprises one or more of microcrystalline cellulose, polyvinylpyrrolidone, copovidone, polyvinylalcohol, starch, gelatin, gum arabic, gum acacia, and gum tragacanth;
said hydrophilic gel-forming polymer of said second mixture comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional lubricant component of said second mixture, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional disintegrant of said second mixture, if present, comprises croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose; and
said optional antioxidant component of said second mixture, if present, comprises one or more of ascorbic acid, sodium ascorbate, ascorbyl palmitate, vitamin E, vitamin E acetate, butylated hydroxytoluene, and butylated hydroxyanisole.

84. The process of claim 82 wherein
said filler/diluent component of said first mixture comprises one or more of lactose or lactose monohydrate;
said filler/binder component of said first mixture comprises microcrystalline cellulose;
said hydrophilic gel-forming polymer component of said first mixture comprises hydroxypropylmethylcellulose;
said optional lubricant component of said first mixture, if present, comprises magnesium stearate;
said filler/diluent component of said second mixture comprises one or more of lactose or lactose monohydrate;
said filler/binder component of said second mixture comprises microcrystalline cellulose;
said hydrophilic gel-forming polymer component of said second mixture comprises hydroxypropylmethylcellulose;
said optional lubricant component of said second mixture, if present, comprises magnesium stearate;
said optional disintegrant of said second mixture, if present, comprises croscarmellose sodium; and
said optional antioxidant component of said second mixture, if present, comprises one or more of ascorbic acid and vitamin E acetate.

85. The process of claim 84 wherein:
said first mixture comprises conjugated estrogens; and
said second mixture comprises one or more therapeutic agents selected from the group consisting of medroxyprogesterone acetate and 1-[4-(2-azepan-1-yl-ethoxy)-benzyl]-2-(4-hydroxy-phenyl)-3-methyl-1H-indol-5-ol, or pharmaceutically acceptable salt thereof.

86. A product of the process of claim 85.
